(19) United States
(12) Patent Application Publication
(10) Pub. No.: US 2026/0308152 A1
(43) Pub. Date: Oct. 8, 2026

(54) METHODS OF MAKING 2-AMINO-1-(3-HYDROXY-2,6-DIMETHYLPHENYL)-5,6-DIMETHYL-1H-PYRROLO-[2,3-B]PYRIDINE-3-CARBOXAMIDE, A MYT1 INHIBITOR

(71) Applicant: Repare Therapeutics Inc., St-Laurent (CA)

(72) Inventors: Angelos DOVLETOGLOU, Hamilton, MA (US); Robert Charles LIVINGSTON, Carlisle, MA (US); Jill A. CAPUTO, Jersey City, NY (US); Sarah M. POUND, East Windsor, NJ (US); Qi GAO, Franklin Park, NJ (US)

(21) Appl. No.: 19/139,501

(22) PCT Filed: Dec. 21, 2023
(86) PCT No.: PCT/CA2023/051731

§ 371 Date: Jun. 16, 2025

Related U.S. Application Data
(60) Provisional application No. 63/434,783, filed on Dec. 22, 2022.

(51) Int. Cl. C07D 471/04; C07B 53/00

(52) U.S. Cl. CPC C07D 471/04; C07B 53/00; C07B 2200/07

(57) ABSTRACT

Disclosed are methods for preparing enantioenriched Compound 1, which involve chiral salt resolution of Compound 1, or of intermediate INT If or intermediate INT 1g. Said Compound 1 is useful for the treatment of a disease or condition (e.g., cancer), in particular those diseases or conditions which depend on the activity of membrane-associated tyrosine and threonine-specific cdc2-inhibitory kinase (Myt1).

### FIELD OF THE INVENTION

The invention relates to synthetic methods and intermediates used for preparing compounds, or pharmaceutically acceptable salts thereof, that are useful for the treatment of a disease or condition, e.g., cancer, and in particular, those diseases or conditions which depend on the activity of membrane-associated tyrosine and threonine-specific cdc2-inhibitory kinase (Myt1).

### BACKGROUND

DNA is continuously subjected to both endogenous insults (e.g., stalled replication forks, reactive oxygen species) and exogenous insults (UV, ionizing radiation, chemical) that can lead to DNA damage. As a result, cells have established sophisticated mechanisms to counteract these deleterious events that would otherwise compromise genomic integrity and lead to genomic instability diseases such as cancer. These mechanisms are collectively referred to as the DNA damage response (DDR). One component of the overall DDR is the activation of various checkpoint pathways that modulate specific DNA-repair mechanisms throughout the various phases of the cell cycle, which includes the G1, S, G2 and Mitosis checkpoints. A majority of cancer cells have lost their G1 checkpoint owing to p53 mutations and as such, rely on the G2 checkpoint to make the necessary DNA damage corrections prior to committing to enter mitosis and divide into 2 daughter cells.

There is a need for convenient synthetic methods that allow for the large scale or commercial scale production of anti-cancer therapeutics, e.g., those that utilizing small molecules, especially therapies allowing for targeted cancer treatment. These synthetic methods can be useful alternatives to existing syntheses, can achieve higher yield, higher isomeric purity (e.g., enantiomeric enrichment), reduced economic costs, or reduced waste stream, or any combination thereof.

### SUMMARY OF THE INVENTION

The invention features methods and intermediates used for preparing an enantioenriched

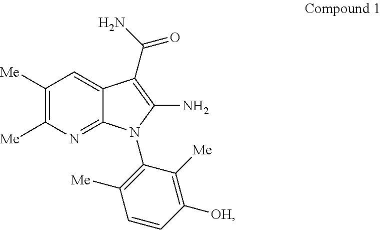 [SMILES: Cc1cc2c(C(N)=O)c(N)n(-c3c(C)ccc(O)c3C)c2nc1C]

In one aspect, the invention provides a method of preparing an enantioenriched Compound 1 or a salt thereof, the method including the step of producing an enantioenriched chiral salt of compound INT 1f:

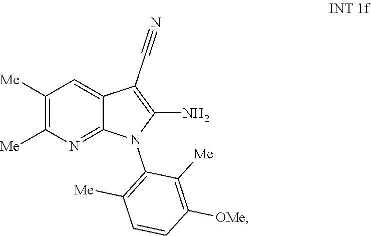 [SMILES: COc1ccc(C)c(-n2c(N)c(C#N)c3cc(C)c(C)nc32)c1C]

and the step of converting the enantioenriched chiral salt of compound INT 1f to Compound 1 or a salt thereof.

In some embodiments, the step of producing an enantioenriched chiral salt includes the step of reacting compound INT 1f with an enantioenriched chiral acid. In some embodiments, the enantioenriched chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; (1S)-(+)-10-Camphorsulfonic acid; (+)-2,3-dibenzoyl-D-tartaric acid; (−)-2,3-dibenzoyl-L-tartaric acid; (−)-O,O′-di-p-toluoyl-L-tartaric acid; (+)-O,O′-di-p-toluoyl-D-tartaric acid; (S)-TCYP; (R)-TCYP; (S)-TRIP; or (R)-TRIP, and the enantioenriched chiral salt is a corresponding chiral acid addition salt of compound INT 1f. In some embodiments, the enantioenriched chiral acid is (1R)-(−)-10-camphorsulfonic acid, and the enantioenriched chiral salt is a (1R)-(−)-10-camphorsulfonic acid salt of compound INT 1f. In some embodiments, the step of reacting compound INT 1f with an enantioenriched chiral acid includes reactive crystallization from anisole.

In some embodiments, the step of converting includes hydrolyzing the enantioenriched salt of compound INT 1f to produce an enantioenriched compound INT 1g:

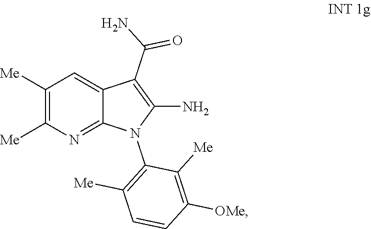 [SMILES: COc1ccc(C)c(-n2c(N)c(C(N)=O)c3cc(C)c(C)nc32)c1C]

or a salt thereof. In some embodiments, the hydrolyzing step includes reacting compound INT 1f with a strong Brønsted acid. In some embodiments, the strong Brønsted acid is methanesulfonic acid. In some embodiments, the step of converting includes deprotecting compound INT 1g or a salt thereof to produce Compound 1 or a salt thereof. In some embodiments, the deprotecting step includes reacting compound INT 1f with methionine.

In some embodiments, compound INT 1g is compound(S)-INT 1g:

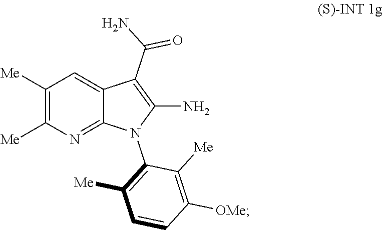

wherein compound INT 1f is compound(S)-INT 1f:

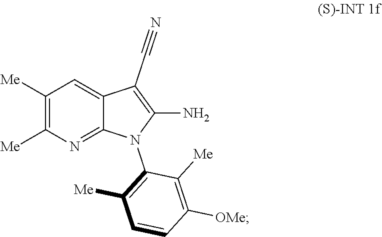

and wherein Compound 1 is (S)-Compound 1:

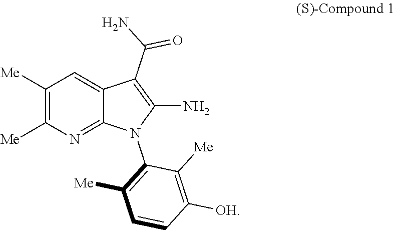

In some embodiments, the step of converting the enantioenriched chiral salt of compound INT 1f to Compound 1 or a salt thereof includes recrystallization. In some embodiments, recrystallization is performed from a methanol/water solvent system.

In another aspect, the invention provides a method of preparing an enantioenriched Compound 1 or a salt thereof, the method including the step of producing an enantioenriched chiral salt of compound INT 1f and the step of converting the enantioenriched chiral salt of compound INT 1g to Compound 1 or a salt thereof.

In some embodiments, the step of producing an enantioenriched chiral salt includes the step of reacting compound INT 1g with an enantioenriched chiral acid. In some embodiments, the enantioenriched chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; (1S)-(+)-10-Camphorsulfonic acid; (+)-2,3-dibenzoyl-D-tartaric acid; (−)-2,3-dibenzoyl-L-tartaric acid; (−)-O,O′-di-p-toluoyl-L-tartaric acid; (+)-O,O′-di-p-toluoyl-D-tartaric acid; (S)-TCYP; (R)-TCYP; (S)-TRIP; or (R)-TRIP, and the enantioenriched chiral salt is a corresponding chiral acid addition salt of compound INT 1g. In some embodiments, the enantioenriched chiral acid is (1R)-(−)-10-camphorsulfonic acid, and the enantioenriched chiral salt is a (1R)-(−)-10-camphorsulfonic acid salt of compound INT 1g.

In some embodiments, the step of reacting compound INT 1g with an enantioenriched chiral acid includes reactive crystallization from anisole. In some embodiments, the step of converting includes deprotecting compound INT 1g or a salt thereof to produce Compound 1 or a salt thereof. In some embodiments, the deprotecting step includes reacting compound INT 1f with methionine. In some embodiments, compound INT 1g is compound(S)-INT 1g and wherein Compound 1 is (S)-Compound 1.

In some embodiments, the step of converting the enantioenriched chiral salt of compound INT 1g to Compound 1 or a salt thereof includes recrystallization. In some embodiments, recrystallization is performed from a methanol/water solvent system.

In yet another aspect, the invention provides a method of preparing an enantioenriched Compound 1, the method including the step of producing an enantioenriched chiral salt of compound 1 and the step of converting the enantioenriched chiral salt of Compound 1 to Compound 1.

In some embodiments, the step of producing an enantioenriched chiral salt includes the step of reacting Compound 1 with an enantioenriched chiral acid. In some embodiments, the enantioenriched chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; (1S)-(+)-10-Camphorsulfonic acid; (+)-2,3-dibenzoyl-D-tartaric acid; (−)-2,3-dibenzoyl-L-tartaric acid; (−)-O,O′-di-p-toluoyl-L-tartaric acid; (+)-O,O′-di-p-toluoyl-D-tartaric acid; (S)-TCYP; (R)-TCYP; (S)-TRIP; or (R)-TRIP, and the enantioenriched chiral salt is a corresponding chiral acid addition salt of Compound 1. In some embodiments, the enantioenriched chiral acid is (1R)-(−)-10-camphorsulfonic acid, and the enantioenriched chiral salt is a (1R)-(−)-10-camphorsulfonic acid salt of Compound 1. In some embodiments, the step of reacting Compound 1 with an enantioenriched chiral acid includes reactive crystallization from anisole.

In some embodiments, Compound 1 is (S)-Compound 1. In some embodiments, the step of converting the enantioenriched chiral salt of Compound 1 to Compound 1 or a salt thereof includes recrystallization. In some embodiments, recrystallization is performed from a methanol/water solvent system.

In some embodiments of any of the aspects above, the enantioenriched chiral acid is at least 90% ee (e.g., at least 95% ee, at least 96% ee, at least 97% ee, at least 98% ee, or at least 99% ee; and up to enantiopure). In some embodiments of any of the aspects above, the enantioenriched Compound 1 or a salt thereof is at least 90% ee (e.g., at least 95% ee, at least 96% ee, at least 97% ee, at least 98% ee, or at least 99% ee; and up to enantiopure). In some embodiments of any of the aspects above, the enantioenriched salt of compound INT 1f is at least 90% ee for compound INT 1f (e.g., at least 95% ee, at least 96% ee, at least 97% ee, at least 98% ee, or at least 99% ee; and up to enantiopure). In some embodiments of any of the aspects above, the enantioenriched salt of compound INT 1g is at least 90% ee for compound INT 1g (e.g., at least 95% ee, at least 96% ee, at least 97% ee, at least 98% ee, or at least 99% ee; and up to enantiopure).

In another aspect, the invention provides a chiral acid addition salt of compound INT 1f, wherein the chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; (1S)-(+)-10-Camphorsulfonic acid; (+)-2,3-dibenzoyl-D-tartaric acid; (−)-2,3-dibenzoyl-L-tartaric acid; (−)-O,O′-di-p-toluoyl-L-tartaric acid; (+)-O,O′-di-p-toluoyl-D-tartaric acid; (S)-TCYP; (R)-TCYP; (S)-TRIP; or (R)-TRIP.

In some embodiments, the chiral acid is (1R)-(−)-10-camphorsulfonic acid.

In some embodiments, compound INT 1f is compound(S)-INT 1f. In some embodiments, the enantioenrichment of compound INT 1f is at least 90% ee (e.g., at least 95% ee, at least 96% ee, at least 97% ee, at least 98% ee, or at least 99% ee; and up to enantiopure).

In another aspect, the invention provides a chiral acid addition salt of compound INT 1g, wherein the chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; (1S)-(+)-10-Camphorsulfonic acid; (+)-2,3-dibenzoyl-D-tartaric acid; (−)-2,3-dibenzoyl-L-tartaric acid; (−)-O,O′-di-p-toluoyl-L-tartaric acid; (+)-O,O′-di-p-toluoyl-D-tartaric acid; (S)-TCYP; or (R)-TCYP. In some embodiments, the chiral acid is (1R)-(−)-10-camphorsulfonic acid.

In some embodiments, compound INT 1g is (S)-INT 1g. In some embodiments, the enantioenrichment of compound INT 1g is at least 90% ee (e.g., at least 95% ee, at least 96% ee, at least 97% ee, at least 98% ee, or at least 99% ee; and up to enantiopure).

In another aspect, the invention provides a chiral acid addition salt of Compound 1, wherein the chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; or (1S)-(+)-10-Camphorsulfonic acid. In some embodiments, the chiral acid is (1R)-(−)-10-camphorsulfonic acid.

In some embodiments, Compound 1 is (S)-Compound 1. In some embodiments, the enantioenrichment of compound INT 1f is at least 90% ee (e.g., at least 95% ee, at least 96% ee, at least 97% ee, at least 98% ee, or at least 99% ee; and up to enantiopure). In some embodiments, the enantioenrichment of compound INT 1g is at least 90% ee (e.g., at least 95% ee, at least 96% ee, at least 97% ee, at least 98% ee, or at least 99% ee; and up to enantiopure). In some embodiments, the enantioenrichment of Compound 1 is at least 90% ee (e.g., at least 95% ee, at least 96% ee, at least 97% ee, at least 98% ee, or at least 99% ee; and up to enantiopure).

### Definitions

As used herein, the term “acid addition salt”, in reference to a compound (e.g., Compound 1, INT-1f, and INT 1g, or an enantiomer thereof, e.g., (S) enantiomer thereof), refers to a salt (e.g., a pharmaceutically acceptable salt) of the compound, in which the compound is protonated and is positively charged, and the counterion is a Brønsted-Lowry conjugate base of an acid (e.g., a chiral acid). Examples of chiral acids include, but are not limited to, the following acids, or isomers thereof:

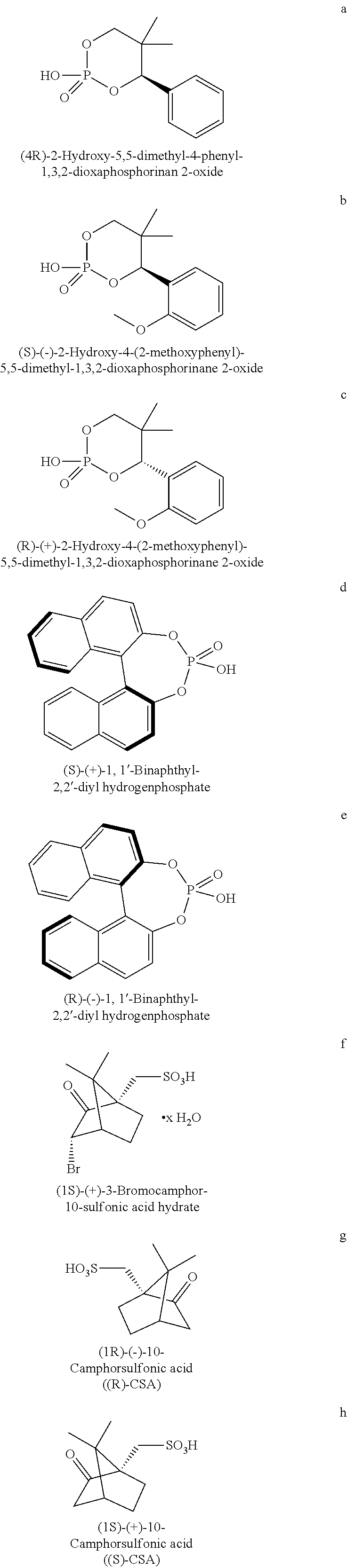

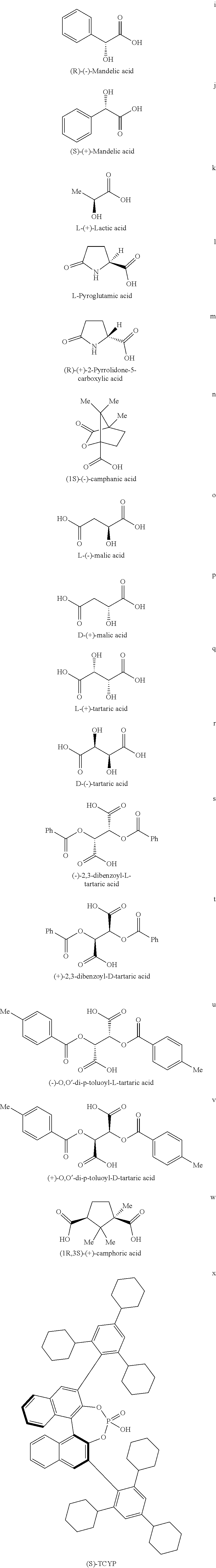

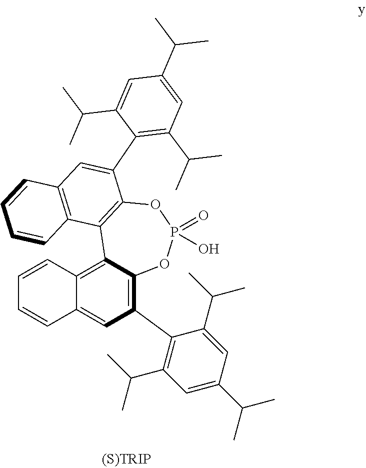

The terms “CCNE1” and “cyclin E1,” as used interchangeably herein, refer to G1/S specific cyclin E1 (Gene name: CCNE1). A cell overexpressing CCNE1 is one that exhibits a higher activity of CCNE1 than a cell normally expressing CCNE1. For example, a CCNE1-overexpressing cell is a cell that exhibits a copy number of at least 3 compared to a diploid normal cell with 2 copies. Thus, a cell exhibiting a copy number greater than 3 of CCNE1 is a cell overexpressing CCNE1. The CCNE1 overexpression may be measured by identifying the expression level of the gene product in a cell (e.g., CCNE1 mRNA transcript count or CCNE1 protein level).

As used herein, the term “Compound 1” refers to a compound having the structure shown below.

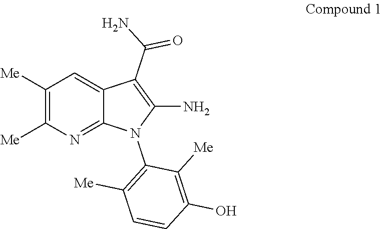 [SMILES: Cc1cc2c(C(N)=O)c(N)n(-c3c(C)ccc(O)c3C)c2nc1C]

Compound 1 may also be referred to as 2-amino-1-(3-hydroxy-2,6-dimethylphenyl)-5,6-dimethyl-1H-pyrrolo-[2,3-b]pyridine-3-carboxamide. Compound 1 can exists as a mixture of both enantiomers, e.g., racemic mixture, or as an enantiomerically enriched mixture of both enantiomers. The two enantiomers of compound 1 are shown below.

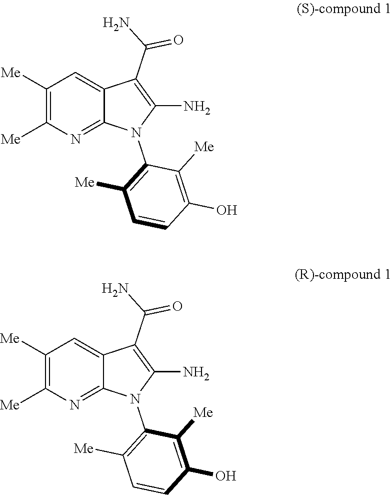

(S)-Compound 1 may also be referred to as(S)-2-amino-1-(3-hydroxy-2,6-dimethylphenyl)-5,6-dimethyl-1H-pyrrolo-[2,3-b]pyridine-3-carboxamide. (R)-Compound 1 may also be referred to as (R)-2-amino-1-(3-hydroxy-2,6-dimethylphenyl)-5,6-dimethyl-1H-pyrrolo-[2,3-b]pyridine-3-carboxamide.

The term “crystalline,” as used herein, refers to a crystalline form of a compound (e.g., Compound 1, INT 1f, and INT 1g) wherein the crystalline form is characterized by a powder X-ray diffraction pattern (XRPD).

The term “Myt1,” as used herein, refers to membrane-associated tyrosine and threonine-specific cdc2-inhibitory kinase (Myt1) (Gene name PKMYT1).

The term “Myt1 inhibitor,” as used herein, represents a compound that upon contacting the enzyme Myt1, whether in vitro, in cell culture, or in an animal, reduces the activity of Myt1.

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The terms “pharmaceutically acceptable excipient” and “pharmaceutically acceptable carrier,” as used interchangeably herein, refer to any ingredient other than the compounds described herein (e.g., a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “tautomer” refers to structural isomers that readily interconvert, often by relocation of a proton. Tautomers are distinct chemical species that can be identified by differing spectroscopic characteristics, but generally cannot be isolated individually. Non-limiting examples of tautomers include ketone-enol, enamine-imine, amide-imidic acid, nitroso-oxime, ketene-ynol, and amino acid-ammonium carboxylate.

### BRIEF DESCRIPTION OF THE DRAWINGS

FIG. 1 is a graph illustrating the solubility of INT 1b in organic solvents, including heptane, toluene, xylenes, isopropyl acetate (IPAc), ethyl acetate (EtOAc), methyl ethyl ketone (MEK), methyl t-butyl ether (MTBE), dichloromethane (DCM), methanol (MeOH), ethanol (EtOH), isopropanol (iPrOH), acetonitrile (MeCN), water, 5 M H₂SO₄, and 1 M H₂SO₄.

FIG. 2 is image depicting the ¹H NMR spectrum of INT 1b from Batch 1—INT 1b as described in Example 2.

FIG. 3 is an ¹H NMR spectrum of INT 1c from Combined Batch 1 and 2—INT 1c as described in Example 3.

FIG. 4 is an ¹H NMR spectrum of INT 1e from Purification—INT 1e as described in Example 4.

FIG. 5 is an ¹H NMR spectrum of INT 1f from Purification—INT 1f as described in Example 5.

FIG. 6A is an ¹H NMR spectrum of INT 1f/Acid d as described in Example 7.

FIG. 6B is an ¹H NMR spectrum of INT 1f/Acid f as described in Example 7.

FIG. 6C is an ¹H NMR spectrum of INT 1f/Acid h as described in Example 7.

FIG. 6D is an ¹H NMR spectrum of INT 1f/Acid t as described in Example 7.

FIG. 6E is an ¹H NMR spectrum of INT 1f/Acid v as described in Example 7.

FIG. 6F is an ¹H NMR spectrum of INT 1f/Acid x as described in Example 7.

FIG. 6G is an ¹H NMR spectrum of INT 1f/Acid y as described in Example 7.

FIG. 7A is an ¹H NMR spectrum of INT 1g/Acid d as described in Example 8.

FIG. 7B is an ¹H NMR spectrum of INT 1g/Acid f as described in Example 8.

FIG. 7C is an ¹H NMR spectrum of INT 1g/Acid h as described in Example 8.

FIG. 7D is an ¹H NMR spectrum of INT 1g/Acid t as described in Example 8.

FIG. 7E is an ¹H NMR spectrum of INT 1g/Acid v as described in Example 8.

FIG. 8A is an ¹H NMR spectrum of Compound 1/Acid d as described in Example 9.

FIG. 8B is an ¹H NMR spectrum of Compound 1/Acid f as described in Example 9.

FIG. 8C is an ¹H NMR spectrum of Compound 1/Acid h as described in Example 9.

FIG. 9A is a chiral HPLC of the solids recovered from reactive crystallization with(S)-CSA using anisole as a solvent, as described in Example 10 (entry 10, Table 13). The earlier elution peak at about 9.1 minutes corresponds to(S)-INT 1f, and the later elution peak at about 10.7 minutes corresponds to (R)-INT 1f.

FIG. 9B is a chiral HPLC of the mother liquor from reactive crystallization with(S)-CSA using anisole as a solvent, as described in Example 10 (entry 10, Table 13). The earlier elution peak at about 9.1 minutes corresponds to(S)-INT 1f, and the later elution peak at about 10.7 minutes corresponds to (R)-INT 1f.

FIG. 10 is an ¹H NMR spectrum of INT 1f/(R)-CSA as described in Example 12.

FIG. 11 is an ¹H NMR spectrum of INT 1f/(S)-CSA as described in Example 12.

FIG. 12 is an ¹H NMR spectrum of (S)-Compound 1 from Batch 1—(S)-INT 1f/(R)-CSA as described in Example 12.

FIG. 13 is a chiral HPLC of (S)-Compound 1, produced as described in Example 12.

FIG. 14A is a chiral HPLC of (S)-Compound 1, from Crystallization 1—(S)-INT 1f/(R)-CSA as described in Example 12.

FIG. 14B is a chiral HPLC of (S)-Compound 1, from Crystallization 2—(S)-INT 1f/(R)-CSA (2^{nd} round, wet cake) as described in Example 12.

FIG. 14C is a chiral HPLC of (S)-Compound 1, from Crystallization 2—(S)-INT 1f/(R)-CSA (3rd round, dried solid) as described in Example 12.

FIG. 15 is an ¹H NMR spectrum of (S)-INT 1f/(R)-CSA from Reactive Crystallization—(S)-INT 1f/(R)-CSA as described in Example 13 (entry 6 in Table 23).

FIG. 16 is an ¹H NMR spectrum of (S)-INT 1f/(R)-CSA from Recrystallization 1—(S)-INT 1f/(R)-CSA as described in Example 13 (entry 2 in Table 24).

FIG. 17A is an ¹H NMR spectrum of crude racemic INT 1f as described in Example 13 (entry 1 in Table 27).

FIG. 17B is an ¹H NMR spectrum of racemic INT 1f after recrystallization in chloroform as described in Example 13 (entry 2 in Table 27).

FIG. 17C is the ¹H NMR spectrum of racemic INT 1f after charcoal (DARCO®) treatment as described in Example 13 (entry 3 in Table 27).

FIG. 18A is a graph illustrating the X-ray powder diffraction (XRPD) patterns for the resulting solids from the racemic INT 1f solubility study as described in Example 14 (Table 28).

FIG. 18B is a graph illustrating the solubility profile of racemic INT 1f and(S)-INT 1f/(R)-CSA in anisole as described in Example 14 (Table 28 and Table 29).

FIG. 19 is a graph illustrating the X-ray powder diffraction (XRPD) patterns for a) racemic INT 1f; b) INT 1f recrystallized from anisole; c) (R)-INT 1f/(S)-CSA; d) (R)-INT 1f/(S)-CSA recrystallized from anisole (wet cake); e) (R)-INT 1f/(S)-CSA recrystallized from anisole (air-dried); and f) (R)-INT 1f/(S)-CSA recrystallized from 1,3-dimethoxybenzene as described in Example 14.

FIG. 20A is an image of the needle crystals of (R)-INT 1f/(S)-CSA recrystallized from anisole.

FIG. 20B is an image of the needle crystals of (R)-INT 1f/(S)-CSA recrystallized from 4-methylanisole.

FIG. 20C is an image of the needle crystals of (R)-INT 1f/(S)-CSA recrystallized from 1,3-dimethoxybenzene.

FIG. 20D is an image of the needle crystals of (R)-INT 1f/(S)-CSA.

FIG. 20E is an image of the needle crystals racemic INT 1f.

FIG. 20F is an image of the needle crystals of INT 1f recrystallized from anisole.

### DETAILED DESCRIPTION

The invention features methods and compounds, or salts thereof, that may be useful for preparing compounds (e.g., the synthesis of Compound 1) useful for the treatment of a disease or condition, e.g., cancer. In particular, the diseases or conditions depend on the activity of membrane-associated tyrosine and threonine-specific cdc2-inhibitory kinase (Myt1). Preferably, Compound 1 is (S)-Compound 1.

The methods and intermediates described herein may be useful for achieving higher yield, higher chemical purity, higher isomeric purity (e.g., enantiomeric enrichment), reduced economic costs, or reduced waste stream, or any combination thereof. The methods of the invention can also enable a reliable and economic, commercial-scale synthesis of Compound 1 (e.g., (S)-Compound 1).

### Compounds

Where possible, the invention includes individual diastereomers, enantiomers, epimers, and atropisomers of the compounds disclosed herein, and mixtures of diastereomers and/or enantiomers thereof including racemic mixtures. Although the specific stereochemistries disclosed herein are preferred, other stereoisomers, including diastereomers, enantiomers, epimers, atropisomers, and mixtures of these may also have utility in treating Myt1-mediated diseases. Inactive or less active diastereoisomers and enantiomers may be useful, e.g., for scientific studies relating to the receptor and the mechanism of activation.

It is understood that certain molecules can exist in multiple tautomeric forms. This invention includes all tautomers even though only one tautomer may be indicated in the examples.

Compounds and intermediates disclosed herein may contain, e.g., one or more stereogenic centers and can occur as racemates, racemic mixtures, single enantiomers, individual diastereomers, and mixtures of diastereomers and/or enantiomers. The invention includes all such isomeric forms of the compounds disclosed herein. It is intended that all possible stereoisomers (e.g., enantiomers and/or diastereomers) in mixtures and as pure or partially purified compounds are included within the scope of this invention (i.e., all possible combinations of the stereogenic centers as pure compounds or in mixtures).

Some of the compounds described herein (e.g., Compound 1, INT 1f, and INT 1g) may contain bonds with hindered rotation such that two separate rotomers, or atropisomers, may be separated and found to have different biological activity which may be advantageous. It is intended that all of the possible atropisomers are included within the scope of this invention.

Some of the compounds described herein may contain olefinic double bonds, and unless specified otherwise, are meant to include both E and Z geometric isomers.

Some of the compounds described herein may exist with different points of attachment of hydrogen, referred to as tautomers. An example is a ketone and its enol form, known as keto-enol tautomers. The individual tautomers as well as mixtures thereof are encompassed by the invention. Compounds disclosed herein having one or more asymmetric centers may be separated into diastereoisomers, enantiomers, and the like by methods well known in the art (e.g., chiral supercritical fluid chromatography (SFC) and/or recrystallization).

Alternatively, enantiomers and other compounds with chiral centers may be synthesized by stereospecific synthesis using enantiomerically pure starting materials and/or reagents of known configuration.

### Acid Addition Salts of Compound 1, INT 1f, and INT 1g

Compound 1, INT 1f, and INT 1g contain bonds with hindered rotation such that two separate atropisomers may be separated. The methods of present invention include techniques for the resolution of the enantiomers of Compound 1, INT 1g, and INT 1f by generating chiral acid addition salts of one or more of the compounds. Preferably, the resolution can be achieved by generating a chiral acid addition salt of INT 1f.

Recrystallization of the chiral acid addition of INT 1f from a solvent system enhances the enantiomeric ratio of INT 1f. Alternatively, when the acid addition salt of INT 1f is generated, a reactive crystallization technique may be employed to enhance the enantiomeric ratio of INT 1f.

Preferably, the methods of the invention are directed to the synthesis of (S)-Compound 1 and employ the use of (S)-INT 1f and(S)-INT 1g.

Chiral acids that may be used in the resolution of any one of Compound 1, INT 1f, and INT 1g include, but are not limited to, the following:

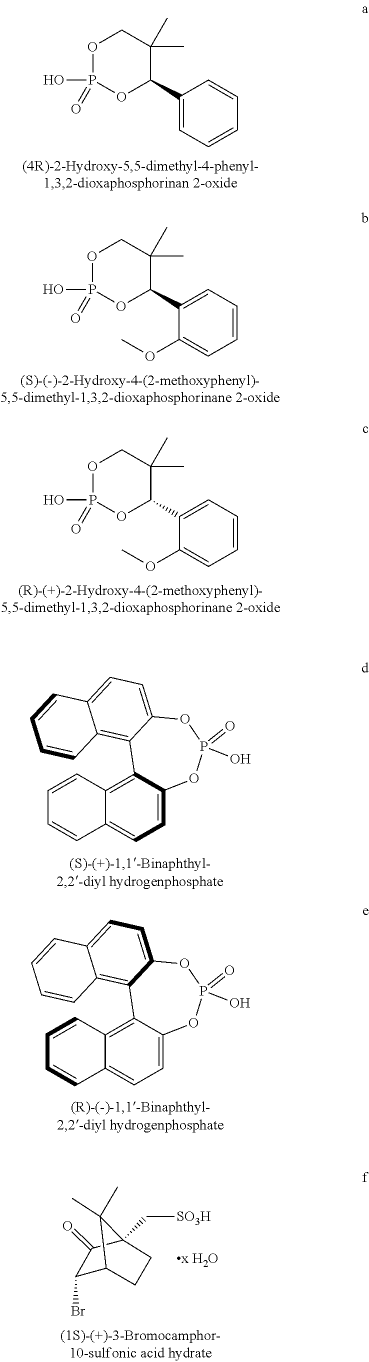

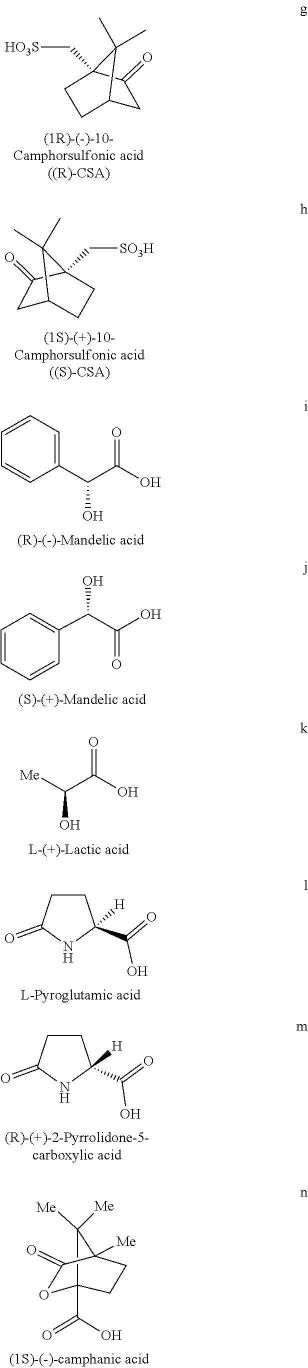

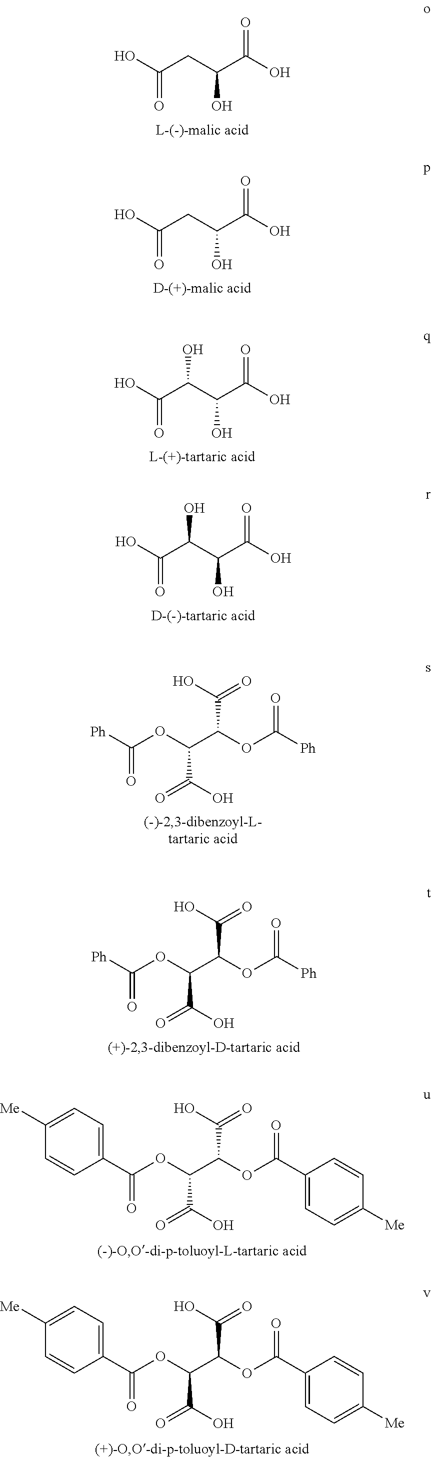

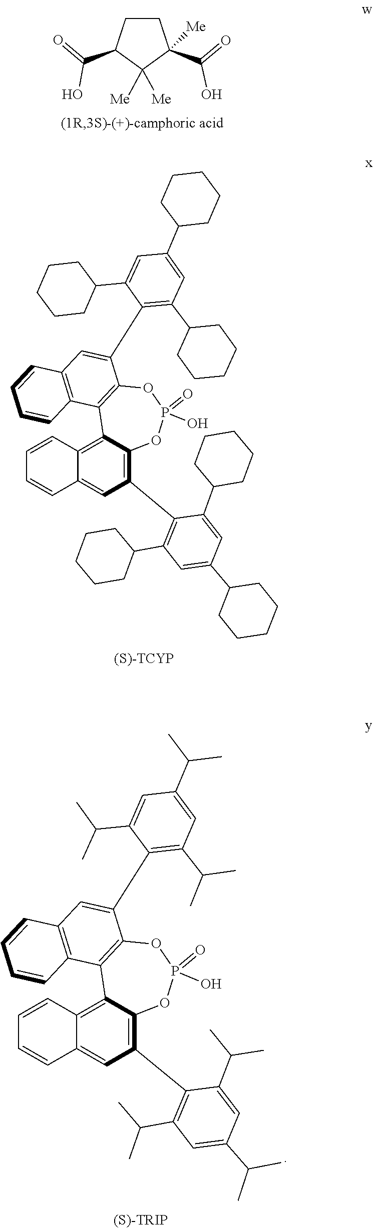

### Myt1

Compounds described herein may be Myt1 inhibitors (e.g., Compound 1) or useful intermediates for synthesizing Myt1 inhibitors (e.g., INT 1f and INT 1g). These compounds may be used to inhibit Myt1 in a cell, e.g., a cell in a subject or to synthesize compounds useful for inhibiting Myt1. The subject may be in need of a treatment for a disease or condition, e.g., a disease or condition having a symptom of cell hyperproliferation, e.g., a cancer. The Myt1 inhibitory activity of compounds disclosed herein may be useful for treating a subject in need of a treatment for cancer.

Myt1 is a cell cycle regulating kinase localized predominantly in the endoplasmic reticulum and golgi complex. It is part of the Wee family of kinases that includes Wee1 and Wee1b. It is involved in the negative regulation of the CDK1-Cyclin B complex which promotes the progression of cells from G2-phase into the mitotic phase (M-phase) of the cell cycle. During DNA damage, Myt1 drives the phosphorylation on CDK1 (both Tyr15 and Thr14 of CDK1) which maintains the kinase complex in an inactive state in G2 as part of the G2 checkpoint response along with Wee1 (which mediates only Tyr15 phosphorylation) and prevents entry into mitosis until the damage has been repaired. Additionally, it has been proposed that Myt1 directly interacts with CDK1 complexes in the cytoplasm and prevents their nuclear translocation thus inhibiting cell cycle progression.

Myt1 has been implicated as a potentially important cancer target as it is essential in many cancer cells. Overexpression of Myt1 has been observed in various cancers including hepatocellular carcinoma as well as clear cell renal cell carcinoma. Myt1 downregulation has a minor role in unperturbed cells but has a more prominent role in cells exposed to DNA damage. Additionally, cells that exhibit high levels of replication stress in addition to defective G1 checkpoint regulation may be particularly sensitive to loss of Myt1 function, as these cells will be prone to entering mitosis prematurely with compromised genomic material leading to mitotic catastrophe.

### EXAMPLES

The following examples are meant to illustrate the invention. They are not meant to limit the invention in any way.

### NMR, HPLC, LC/MS

NMR data were collected on a 400 MHz Bruker Avance III HD instrument equipped with a 5 mm broad band auto tuneable multinuclear probe with z-axis gradient.

A general achiral HPLC method is shown below.

Column: Agilent Poroshell EC-C18, 2.7
μm, 4.6 × 100 mm, PN: 695975-902(T)

| | |
|---|---|
| MPA: 0.025% TFA in H₂O | MPB: 0.025% TFA in CH₃CN |
| UV: 215 nm | Flow rate: 1.0 mL/min |
| Column temp.: — | Injection Volume: 3 μL |

Diluent: 80% ACN w/0.025% TFA, 20% H₂O w/0.025% TFA

| Time (min) | % B |
|---|---|
| 0.0 | 10 |
| 17.0 | 90 |
| 22.0 | 90 |
| 22.1 | 10 |
| 27.0 | 10 |

Impurities elute before 17 mins. All intermediates are well separated. MPB % at 90% does not need to be kept for 5 min, it can be shortened to 1 min, and final equilibration time (MPB 10% from 22.1 to 27 mins) can be cut down to two or three mins.

Diluent and suggested sample concentration for each intermediate of the examples is shown below.

| Intermediate | Retention | | Suggested | |
|---|---|---|---|---|
| No. | time (min) | Diluent | conc. | |
| INT 1d | 5.1 | CH₃CN/water 50/50 | 0.15 mg/mL | |
| | | or 70/30 | to 0.2 ng/mL | |
| INT 1c | 12.7 | CH₃CN/water 70/30 | 0.5 | mg/mL |
| INT 1e | 11.0 | CH₃CN | 0.15 | mg/mL |
| INT 1f | 12.1 | CH₃CN/water 70/30 | 0.125 | mg/mL |
| INT 1g | 7.8 | CH₃CN/water 70/30 | 0.15 | mg/mL |
| Compound 1 | 5.8 | CH₃CN/water 70/30 | 0.15 | mg/mL |

A general chiral HPLC method for INT 1f is shown below.

Column: Lux Cellulose-4, 4.6 × 150 cm, 5 μm

| | |
|---|---|
| Mobile Phase A: Heptane | Diluent: EtOH/Heptane (1/1) |
| Mobile Phase B: EtOH | |
| Mobile phase: A/B = 92/8 | |
| Detector: 220 nm | Flow Rate: 1.0 mL/min |
| Injection volume: 5 μL | Column Temperature: 25° C. |
| Sample Conc.: 1.0 mg/mL | Run Time: 15 min |

A general chiral HPLC method for INT 1g is shown below.

Column: Lux Cellulose-4, 4.6 × 150 cm, 5 μm

| | |
|---|---|
| Mobile Phase A: Heptane | Diluent: EtOH/Heptane (3/7) |
| Mobile Phase B: EtOH | |
| Mobile Phase C: MeOH | |
| Mobile phase: A/B/C = 70/24/6 | |
| Detector: 226 nm | Flow Rate: 1.0 mL/min |
| Injection volume.: 5 μL | Column Temperature: 25° C. |
| Sample Conc.: 1.0 mg/mL | Run Time: 15 min |

A general chiral HPLC method for Compound 1 is shown below.

Column: Lux Cellulose-4, 4.6 × 150 cm, 5 μm

| | |
|---|---|
| Mobile Phase A: Heptane | Diluent: EtOH/Heptane (1/1) |
| Mobile Phase B: EtOH | |
| Mobile phase: A/B = 70/30 | |
| Detector: 226 nm | Flow Rate: 1.0 mL/min |
| Injection volume.: 5 μL | Column Temperature: 40° C. |
| Sample Conc.: 1.0 mg/mL | Run Time: 15 min |

A general LC/MS method is shown below.

Column: Kinetex 5 μm C18 100 Å,
4.6 × 150 mm, PN: 00F-4601-E0

| | |
|---|---|
| MPA: 0.05% Formic acid in H₂O | MPB: 0.05% Formic acid in CH₃CN |
| Flow rate: 1.5 mL/min | Column temp.: 30° C. |

| Time (min) | B % |
|---|---|
| 0 | 10 |
| 6 | 95 |
| 8 | 95 |
| 10 | 10 |

### Abbreviations

Abbreviations and terms that are commonly used in the fields of organic chemistry, medicinal chemistry, pharmacology, and medicine and are well known to practitioners in these fields are used herein. Representative abbreviations and definitions are provided below:
ACN Acetonitrile
2-MeTHF 2-Methyltetrahydrofuran
DME 1,2-Dimethoxyethane or ethylene glycol dimethyl ether
DMF Dimethyl formamide
EtOAc Ethyl acetate
EtOH Ethanol
HPLC High performance liquid chromatography
LCAP Liquid chromatography area percent
MSA Methanesulfonic acid
IPC In-process control
Pd(dppf)Cl₂·CH₂Cl₂ [1,1-Bis(diphenylphosphino) ferrocene]dichloropalladium (II), complex with dichloromethane
Pd₂(dba)₃ Tris(dibenzylideneacetone) dipalladium (0)
Pd(OAc)₂ Palladium acetate
i-PrOH 2-Propanol
(R)-CSA (1R)-(−)-10-Camphorsulfonic acid
(S)-CSA (1S)-(+)-10-Camphorsulfonic acid
TBME t-Butylmethyl ether
THF Tetrahydrofuran
Xantphos 4,5-Bis(diphenylphosphino)-9,9-dimethylxanthene

### Example 1. Preparation of (S)-Compound 1

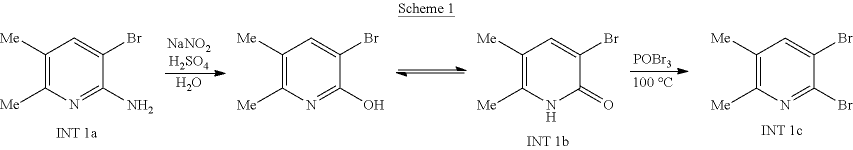

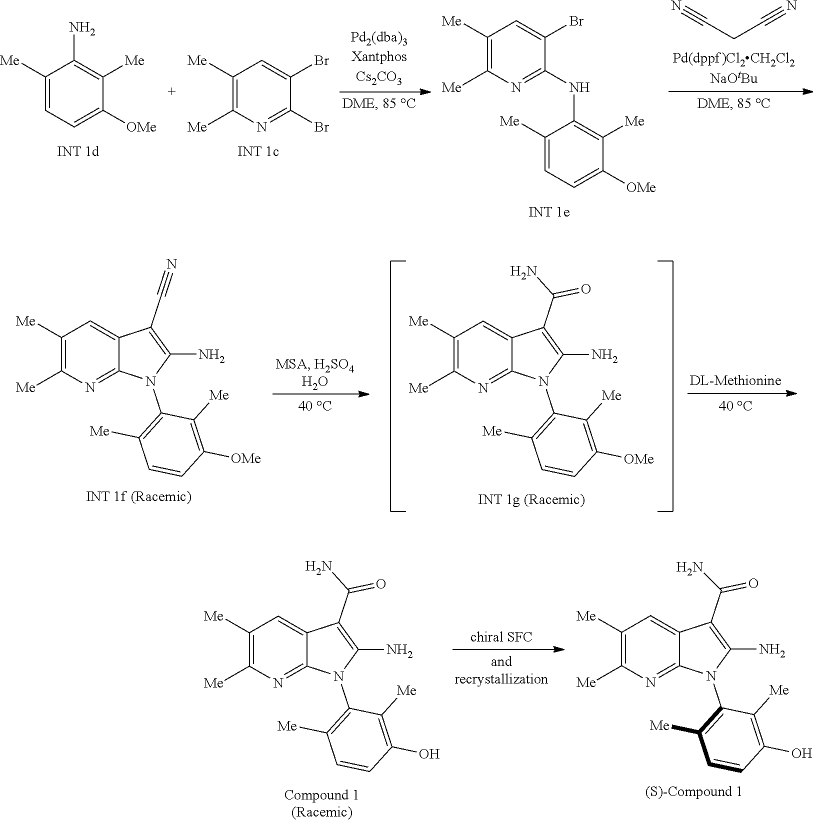

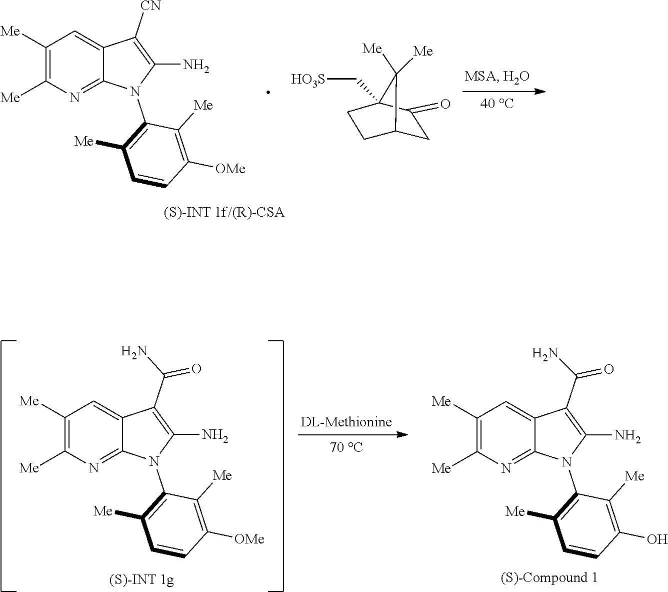

INT 1f, INT 1g, and Compound 1 are atropisomeric. The desired enantiomer of Compound 1 has been isolated by chiral SFC (Scheme 1) and assigned as the(S) enantiomer. Although, resolution by chiral SFC was amenable for isolating gram quantities of the desired enantiomer, it is inefficient for delivering kilograms of material.

Consequently, a chiral salt resolution of late-stage intermediate INT 1f, has been developed. From twenty-five chiral acids that were screened, seven were take forward for further assessment based on whether a salt was formed. INT 1f is a weak base and as a consequence formed salts with chiral sulfonic acids as determine by chemical shifts in the ¹H NMR signals for the pyridine 4H proton (downfield 0.5 to 0.6 ppm) and methyl protons on the pyridine ring (down field 0.2 to 0.3 ppm). The other salts chosen were based on the observation that resolution of some of the proton signals were observed in their corresponding spectra. From the seven acids screened, it was found that the enriched salt formed from INT 1f and (R)-CSA when processed downstream gave the enantiomerically enriched(S)-Compound 1 without chiral erosion. The INT 1f and (R)-CSA salt was prepared from a reactive crystallization in anisole between mole equivalent amounts of INT 1f and (R)-CSA to give enantiomerically enriched salt (85:15 e.r.) in 37% yield (74% recovery of available enantiomer). A recrystallization from anisole gave further enriched salt (95:5 e.r.) in 30% overall yield for the two-step process.

### Example 2. Preparation of INT 1b

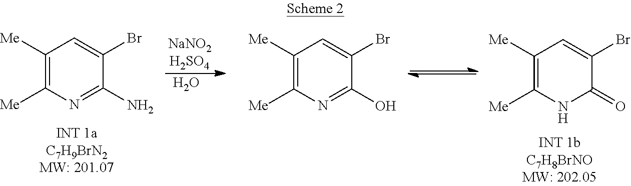 [SMILES: Cc1cc(Br)c(N)nc1C ; O ; Cc1cc(Br)c(O)nc1C ; Cc1cc(Br)c(=O)[nH]c1C]

INT 1b was accessed through the diazotization of INT 1a. Initial conditions involved adding concentrated H₂SO₄ to a suspension of INT 1a in water, which was highly exothermic, and the subsequent addition of NaNO₂ led to violent off gassing. The reaction became more manageable for scale up by the addition of solid INT 1a to a pre-made solution of 1 M H₂SO₄, which was not exothermic, and the rate of off gassing was controlled by the slow addition of an aqueous solution of NaNO₂. INT 1a was initially soluble in the acidic solution at 10° C., but upon addition of NaNO₂, solids immediately began to precipitate.

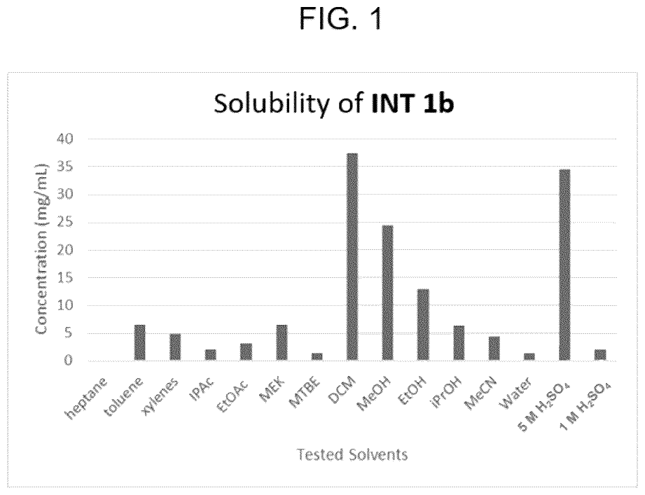

INT 1b has poor solubility in most organic solvents (see FIG. 1). As a result, the reaction was quenched with 2 M K₂HPO₄ to precipitate INT 1b.

The reaction was run on 300 g scale (Batch 1—INT 1b) in the Kilo Lab in a 20 L reactor. After NaNO₂ addition and aging at 10-20° C. for 1 h, the reaction was determined to be complete (INT 1a not detected by HPLC). The suspension was quenched with 2 M aq. K₂HPO₄, and the solids were filtered and washed with H₂O to give INT 1b.

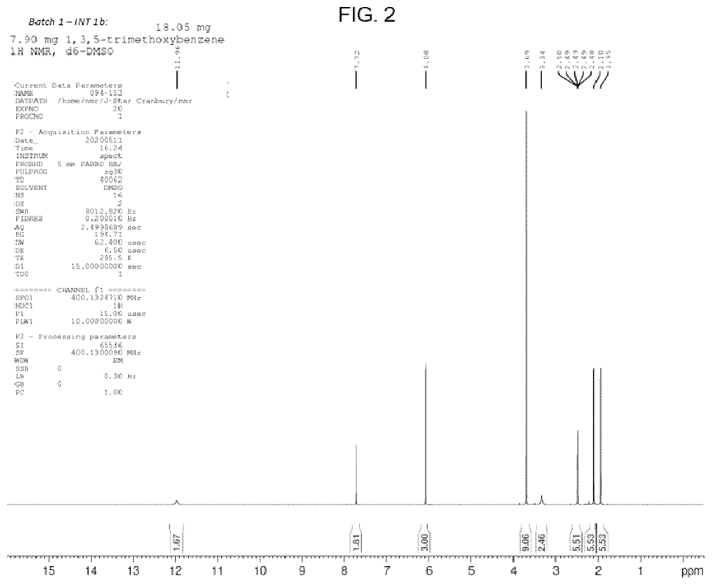

Batch 1—INT 1b: To a 20 L jacketed reactor with stir bearing, thermocouple, N₂ inlet, and NaOH scrubber was charged aqueous H₂SO₄ (1 M, 1.50 L, 1.53 kg). To this was charged INT 1a (299.2 g, 1.49 mol) as a solid. The suspension was diluted with aqueous H₂SO₄ (1 M, 1.50 L, 1.62 kg) and cooled to 0-5° C. Aqueous NaNO₂ (100 mg/mL, 1.34 L, 1411.8 g) was charged dropwise via addition funnel over 2 h maintaining Tᵢₙₜₑᵣₙₐₗ≤10° C. The reactor was warmed to 10-20° C. The suspension was aged at 10-20° C. for 1 h at which point the reaction was complete by HPLC analysis (INT 1a not detected). The reactor was cooled to 0-5° C. The reaction was quenched by dropwise addition of aqueous K₂HPO₄ (2 M, 3.0 L, 4022.2 g) to pH=6. The solids were filtered under vacuum (reactor was emptied with a filtrate swish) and slurry-washed with H₂O (2×3.0 L). The solid was allowed to dry on the filter overnight then transferred to a drying tray and dried in the hood with air flow overnight then under vacuum at 40° C. for 2 days to afford INT 1b as a tan solid (270.3 g, 97.6 area %, 96.4 wt %=260.6 g, 1.29 mol) in 86.4% isolated yield. The 1H NMR spectrum of INT 1b is shown in FIG. 2.

### Example 3. Preparation of INT 1c

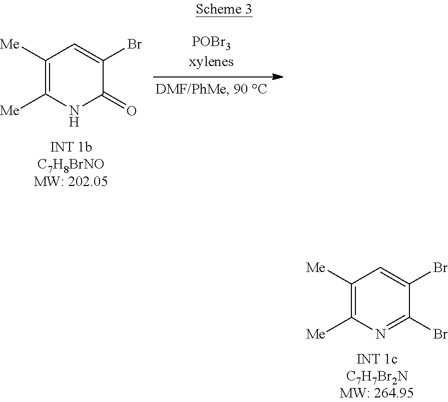 [SMILES: Cc1cc(Br)c(=O)[nH]c1C ; Cc1cc(Br)c(Br)nc1C]

INT 1b was initially reacted with POBr₃ in sulfolane at 105° C., and the reaction required at least 2.5 mol equiv. of POBr₃ in order to drive the reaction to near completion in 8 h (Table 1).

TABLE 1
Charge of POBr₃

| | |
|---|---|
| POBr₃ | LCAP |

| Entry | mol eq. | INT 1b | INT 1c |
|---|---|---|---|
| 1 | 0.625 | 30.9 | 43.1 |
| 2 | 1.50 | 13.0 | 65.9 |
| 3 | 2.50 | 2.3 | 75.4 |

Rather than solid POBr₃, POBr₃ in xylenes was employed and the reaction was performed in dimethylformamide (DMF). Charging POBr₃ in xylenes over 1 h to a solution of INT 1b in DMF led to the formation of a black sludge that was difficult to analyze. Consequently, various solvents were investigated for the reaction (Table 2). In aprotic solvents such as DMF or dimethylacetamide (DMAc), the reaction proceeded to completion (<0.2 area % INT 1b). However, numerous side products were observed. In ethereal solvents such as THF and dioxane, the reaction did not go to completion. When the reaction was run in straight xylenes the reaction profile was much cleaner, and INT 1c was obtained in 89.7 area %.

TABLE 2
Solvent Screen

LCAP

| Entry | Solvent | T (° C.) | INT 1b | INT 1c |
|---|---|---|---|---|
| 1 | DMF | 105 | nd¹ | 61.7 |
| 2 | DMAc | 105 | nd¹ | 61.2 |
| 3 | xylenes | 105 | 4.5 (soln.) | 9.5 (soln.) |
| | | | 5.4 (solid)² | 89.7 (solid)² |
| 4 | THF | 50 | 51.7 | 15.0 |
| 5 | dioxane | 100 | 6.0 | 62.7 |

¹nd = not detected
²Reaction was heterogeneous. Aliquot for analysis was filtered and separate HPLC runs were conducted of the solid and solution.

While the reaction in xylenes gave a cleaner reaction compared to other solvents, the reaction did not go to completion. The addition rate of POBr₃ over 2 h (slow addition) and 0.5 h (fast addition) was investigated (Table 3). The addition rate did not have a strong impact on reaction conversion. INT 1b went into solution at 90° C. in xylenes and at 70° C. in toluene, leading to a homogeneous solution prior to POBr₃ addition. Therefore, in an attempt to enhance the reaction profile and drive the reaction to completion, mixtures of solvents were investigated (Table 4). Of the solvent systems screened, bromination in DMF/toluene gave the best results in terms of completion (<0.2 area % INT 1b) and reaction profile giving INT 1c in 72 area % purity.

TABLE 3
Rate of Addition of POBr₃

| | | | | LCAP | |
|---|---|---|---|---|---|
| Entry | Scale | Solvent | POBr₃ | INT 1b | INT 1c |
| 1 | 2 g | xylenes | 1.5 eq, | 52.0 (soln.) | 24.0 (soln.) |
| | INT 1b | | fast addition | 11.3 (solid)² | 87.2 (solid)² |
| 2 | 5 g | xylenes | 2.5 eq, | 37.1 (soln.) | 4.3 (soln.) |
| | INT 1b | | slow addition | 16.6 (solid)² | 82.2 (solid)² |
| 3 | 5 g | toluene | 2.5 eq, | 0.3 (soln.) | nd¹ (soln.) |
| | INT 1b | | slow addition | 6.2 (solid)² | 93.8 (solid)² |

¹nd = not detected.
²Reaction was heterogeneous. Aliquot for analysis was filtered and separate HPLC runs were conducted of the solid and solution.

TABLE 4
Solvent Screening Continued, Mixtures of Solvents

LCAP

| Entry | Solvent | T (° C.) | INT 1b | INT 1c |
|---|---|---|---|---|
| 1 | 3:5 DMF/toluene | 100 | nd¹ | 72.0 |
| 2 | 3:5 DMF/dioxane | 100 | 30.0 | 53.5 |
| 3 | 3:5 dioxane/toluene | 100 | 10.2 | 19.7 |

¹nd = not detected

Using the improved conditions, the reaction was run twice at the 100 g batch scale (Batch 1-INT 1c and Bath 2—INT 1c). Batch 1—INT 1c afforded INT 1c in 84.5% isolated yield and Batch 2—INT 1c afforded INT 1c in 70.8% isolated yield. The two batches were combined and recrystallized from TBME/heptane (Combined Batch 1 and 2—INT 1c), and INT 1c was isolated as a tan solid in 84.0% recovery.

Batch 1—INT 1c: To a 3-neck 2 L round-bottom flask with overhead stirring, thermocouple, nitrogen inlet, and NaOH scrubber was charged DMF (150 mL). To the solvent was charged INT 1b (103.7 g, lot 094-153). The suspension was diluted with DMF (150 mL) and heated to 90° C. Toluene (500 mL) was charged to the solution. POBr₃ (379 g, 56.5 wt % solution in xylenes) was charged to the solution over 90 minutes (solids formed at the site of addition and on the sides of the flask). The reaction was heated at 90° C. with vigorous stirring for 16 h at which point the reaction was complete by HPLC analysis (no INT 1b detected, 96.4 area % INT 1c). The crude reaction mass was cooled to ambient temperature then poured portion-wise into two 2 L round-bottom flasks each containing 1.0 L water and cooled with an ice/water bath. The reaction vessel was washed with water (500 mL), which was added to the quenching vessels. Each quench was worked up by: (1) extracting the aqueous layer with TBME (3×500 mL); (2) combining the organics and washing with aq. NaOH (0.5 N, 500 mL) and water (500 mL); and (3) drying the organics over Na₂SO₄ (100 g). The organics from both extractions were combined by filtering into a 2 L round-bottom flask. The Na₂SO₄ was washed with TBME (100 mL). The filtrate was concentrated to dryness under reduced pressure to give crude INT 1c as an orange solid (124.8 g, 97.5 area %, 88.8 wt % by HPLC vs. working standard=110.8 assay g, 418.3 mmol) in 84.5% recovery.

Batch 2—INT 1c: This procedure was repeated on the same scale (103.7 g INT 1b, lot 094-153). At 16 h, the reaction was complete by HPLC analysis (no INT 1b detected, 95.9 area % INT 1c). Aqueous workup afforded INT 1c as an orange solid (126.2 g, 94.4 area %, 73.6 wt % by HPLC vs. working standard=92.9 assay g, 350.6 mmol) in 70.8% isolated yield.

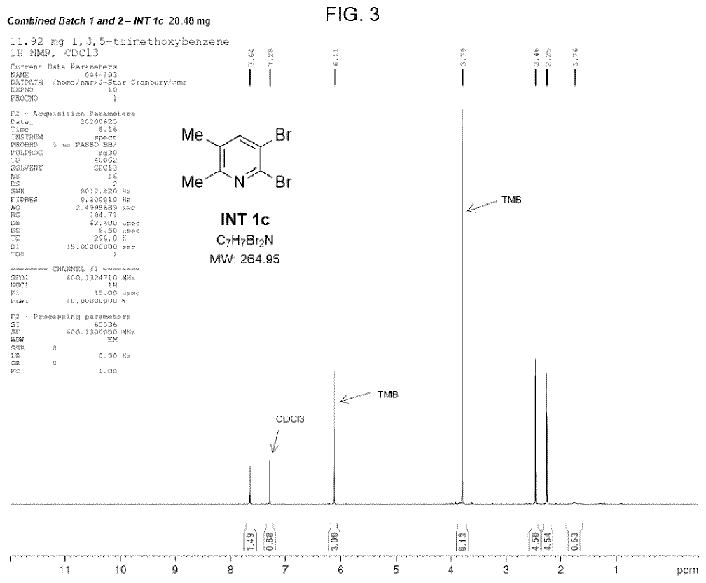

Combined Batch 1 and 2—INT 1c: A suspension of INT 1c (combined lots 094-188:124.8 g, 97.5 area %, 88.8 wt % by HPLC vs. working standard=110.8 assay g, 418.3 mmol; 094-191:126.2 g, 94.4 area %, 73.6 wt % by HPLC vs. working standard=92.9 assay g, 350.6 mmol) in TBME (1.0 L, 4 vols) was heated to ~60° C. to dissolve most of the solids. Heptane (750 ml, 3 vols) was charged, and the suspension was slowly cooled to ambient temperature and then concentrated to ~1.65 vol under reduced pressure over 3 h. The solid was collected by filtration, washed with heptane (250 mL), and dried on the filter under a nitrogen atmosphere to constant weight to afford INT 1c as a tan colored solid (214.3 g, 99.3 area %, 98.4 wt % by ¹H NMR vs. 1,3,5-trimethoxybenzene internal standard=210.9 assay g, 796.0 mmol) in 84.0% recovery. The ¹H NMR spectrum of the purified INT 1c is shown in FIG. 3.

### Example 4. Preparation of INT 1e

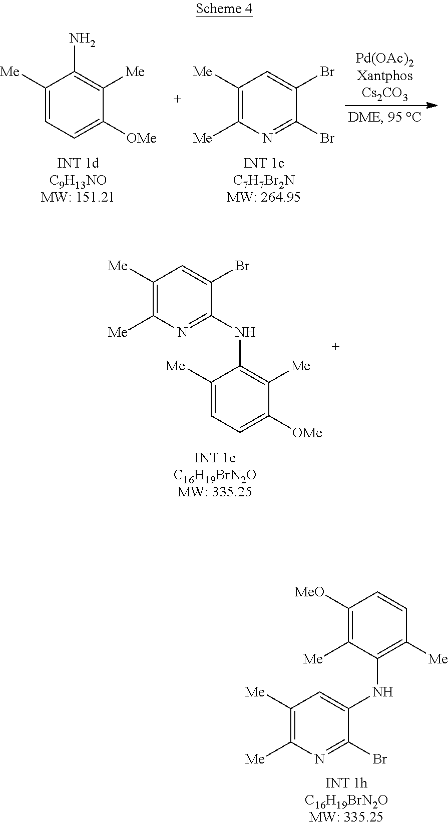 [SMILES: COc1ccc(C)c(N)c1C ; Cc1cc(Br)c(Br)nc1C ; COc1ccc(C)c(Nc2nc(C)c(C)cc2Br)c1C ; COc1ccc(C)c(Nc2cc(C)c(C)nc2Br)c1C]

Reaction conditions including Pd(OAc)₂, Xantphos, Cs₂CO₃, and DME at 95° C. were investigated. The reaction screen was performed on small scale (100 mg INT 1d) at reflux temperature, and some loss of solvent occurred. Consequently, it was difficult to get an accurate assay yield as the final reaction volumes were unknown, so reactions were compared by area % of INT 1e rather than solution assay yields. The use of rac-BINAP in place of Xantphos led to incomplete conversion. Varying solvent, temperature, and base did not offer any significant improvement over the current conditions (Table 5).

TABLE 5
Initial Screen, Pd(OAc)₂ as catalyst 5 mol %

| | | | | | LCAP | | |
|---|---|---|---|---|---|---|---|
| | | | | T | INT | INT | INT |
| Entry | Ligand | Base | Solvent | (° C.) | 1d | 1c | 1e |
| 1 | Xantphos | Cs₂CO₃ | DME | 85 | nd¹ | 0.5 | 70.8 |
| 2 | Rac-BINAP | Cs₂CO₃ | DME | 85 | 7.2 | 7.1 | 66.3 |
| 3 | Xantphos | Cs₂CO₃ | Dioxane | 85 | nd¹ | 0.3 | 68.5 |
| 4 | Xantphos | Cs₂CO₃ | Toluene | 110 | nd¹ | 0.8 | 62.9 |
| 5 | Xantphos | Cs₂CO₃ | DMF | 110 | nd¹ | nd¹ | 37.1 |
| 6 | Xantphos | Cs₂CO₃ | DME | 70 | 4.4 | 0.3 | 67.1 |
| 7 | Xantphos | K₂CO₃ | DME | 85 | nd¹ | 8.8 | 60.4 |
| 8 | Xantphos | NaO^{t}Bu | DME | 85 | 7.4 | nd¹ | 72.3 |
| 9 | PPh₃ | NaO^{t}Bu | Xylenes | 120 | 8.8 | nd¹ | 64.4 |

¹nd = not detected.

The use of Pd₂(dba)₃ in place of Pd(OAc)₂ improved the reaction and led to complete consumption of INT 1c with a small amount of INT 1d remaining (Table 6, entry 2). Given the possibility that INT 1c was being consumed and forming byproducts (i.e., des-bromo lutidine), we increased the charge of INT 1c and saw a further improvement (Table 6, entry 3).

TABLE 6
Additional Screening Pd₂(dba)₃ as catalyst versus Pd(OAc)₂

| | | | | | LCAP | | |
|---|---|---|---|---|---|---|---|
| | Pd, | Base, | Equiv. | Time | INT | INT | INT |
| Entry | mol % | equiv. | INT 1c | (h) | 1d | 1c | 1e |
| 1 | Pd(OAc)₂, | Cs₂CO₃, | 1.0 | 18 | nd¹ | 0.5 | 70.8 |
| | 10 mol % | 4.0 equiv. | | | | | |
| 2 | Pd₂(dba)₃, | Cs₂CO₃, | 1.0 | 18 | 1.8 | nd¹ | 77.8 |
| | 5 mol % | 4.0 equiv. | | | | | |
| 3 | Pd₂(dba)₃, | Cs₂CO₃, | 1.1 | 3 | 1.6 | 3.1 | 84.0 |
| | 5 mol % | 4.0 equiv. | | 5 | 0.6 | 1.5 | 85.5 |
| 4 | Pd(OAc)₂, | NaO^{t}Bu, | 1.1 | 3 | 7.4 | 4.6 | 68.1 |
| | 10 mol % | 3.0 equiv. | | 5 | 4.6 | 1.1 | 75.7 |
| 5 | Pd₂(dba)₃, | NaO^{t}Bu, | 1.1 | 4 | 16.5 | 10.7 | 56.2 |
| | 5 mol % | 3.0 equiv. | | 6.5 | 14.4 | 7.3 | 64.2 |

¹nd = not detected.

The two best conditions (entries 3 and 4) were scaled up to compare solution assay yields. These reactions were run using 0.50 g INT 1d (1.0 equiv.) and 0.96 g INT 1c (1.1 equiv.) in DME (10 vols) at 85° C. with 10 mol % Xantphos as the ligand. At 4.5-5 h, both reactions were complete and were filtered for solution assay yields. The reaction with 10 mol % Pd(OAc)₂ and 3.0 mol eq of NaO Bu afforded INT 1e in 68% yield, while the yield for the reaction with 5 mol % Pd₂(dba)₃ and 4.0 mol eq Cs₂CO₃ was 86%. From these results, the reaction was scaled up using Pd₂(dba)₃ and Cs₂CO₃ (Batch 1—INT 1e) with 20.2 g INT 1d and 43.1 g INT 1c. After heating at reflux for 15 h, the reaction was determined to be complete (<1.5 area % INT 1d remaining). Workup afforded INT 1e in 87.6% isolated yield. The batch was recrystallized from 2-isopropanol (Recrystallization 1—INT 1e), and INT 1e was isolated as a yellow solid in 95.1% recovery.

Batch 1—INT 1e: To a 3-neck 1 L round-bottom flask with overhead stirring, thermocouple, nitrogen inlet, and reflux condenser were charged DME (200 mL, sparged prior to use) and INT 1d (20.18 g, 99.1 wt % by QNMR vs. mesitylene standard, 20.0 assay g, 132.3 mmol). To the resulting solution were charged INT 1c (Combined Batch 1 and 2—INT 1c, 43.07 g, 89.5 wt % by HPLC vs. working standard, 36.6 assay g, 145.5 mmol), Cs₂CO₃ (172.4 g, 529.1 mmol), Xantphos (7.65 g, 13.2 mmol), and Pd₂(dba)₃ (6.06 g, 6.61 mmol). The suspension was diluted with DME (200 mL, sparged prior to use). The headspace was flushed with nitrogen, and the reaction was heated to reflux (~85° C.). At 15 h the reaction was cooled to ambient temperature and filtered through a plug of Solka-Floc with EtOAc (500 mL). The Solka-Floc was washed with EtOAc (200 mL). Solution assay yield=83.9%. The filtrate and wash were passed through a 0.45 μm polishing filter and concentrated to low volume (~1-2 vol). Heptane (80 mL) was added dropwise, and the mixture was stirred at 0-10° C. for 1 h. The solids were filtered, washed with cold 5% EtOAc/heptane (60 mL), and dried on the filter under N₂ for overnight to afford INT 1e as a tan solid (51.45 g, 75.9 area %, 75.5 wt % by HPLC vs. working standard=38.8 assay g, 115.7 mmol) in 88% yield.

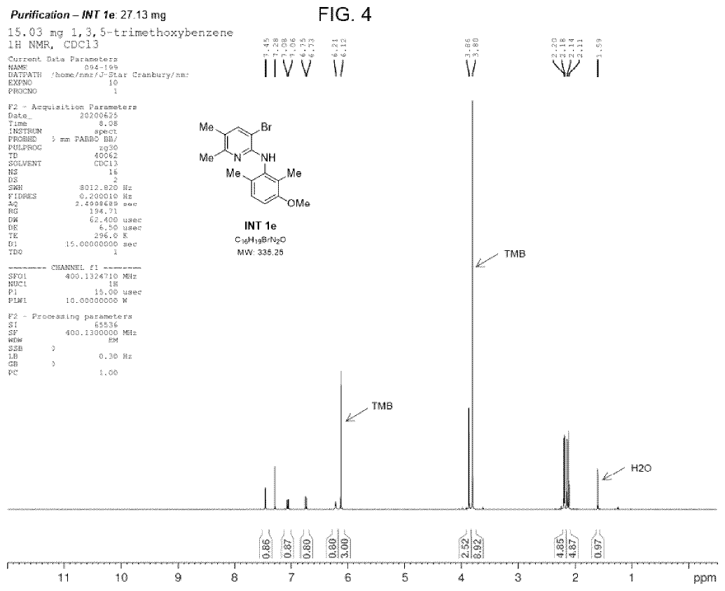

Purification—INT 1e: A suspension of INT 1e (Batch 1—INT 1e, 51.45 g, 75.9 area %, 75.5 wt %=38.8 assay g, 115.7 mmol) in 2-isopropanol (310 mL, 8 vols), was heated to reflux temperature (~81° C.) for 30 min to dissolve the solids, and then cooled slowly to give a suspension. After stirring for ~1 h at ambient temperature, the solid was collected by filtration, washed with cold 2-isopropanol (58 mL), and dried overnight on the funnel under a nitrogen atmosphere. INT 1e was isolated as a yellow solid (36.88 g, 96.3 area %, 95.0 wt % by ¹H NMR vs. 1,3,5-trimethoxybenzene internal standard=35.0 assay g, 104.5 mmol) in 90.3% recovery, and in 78.8% assay yield from INT 1d. The ¹H NMR spectrum of the purified INT 1e is shown in FIG. 4.

### Example 5. Preparation of INT 1f (Racemic)

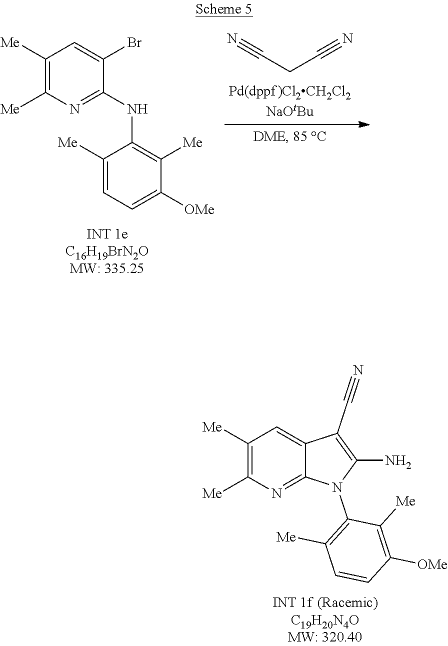 [SMILES: COc1ccc(C)c(Nc2nc(C)c(C)cc2Br)c1C ; N#CCC#N ; COc1ccc(C)c(-n2c(N)c(C#N)c3cc(C)c(C)nc32)c1C]

When the azaindole formation was performed on 1.0 g of INT 1e (Purification—INT 1e), only minimal formation of INT 1f was seen after 5 h at reflux (87 area % INT 1e, 5.3 area % INT 1f). Overnight reflux did not significantly improve the conversion (8.7 area % INT 1f). To determine whether the sluggish reaction was due to the presence of water, KF titrations were conducted on the reagents. NaO Bu solid (fresh bottle, open one day) had the highest water content (Table 7). So, the azaindole formation was tested using NaO Bu solution (2M in THF) on 1.0 g scale of INT 1e. After 5 h at reflux temperature, the reaction was over ~50% conversion (15 area % INT 1e, 69 area % INT 1f).

TABLE 7
Reagent water content

| Reagent | H₂O (wt %) |
|---|---|
| NaO^{t}Bu, solid | 2.55 |
| NaO^{t}Bu, 2M soln. in THF | 0.54 |
| INT 1e | 1.64 |
| Malononitrile | 0.05 |
| DME | 0.35 |

As use of a 2 M solution of NaOtBu in THF worked well, it was used for subsequent INT 1f preparation (Batch 1—INT 1f) on 34.4 g scale of INT 1e. The reaction was determined to be complete after refluxing 16 h (1.5 area % INT 1e remaining). Extractive workup afforded INT 1f in 71.8% isolated yield. To obtain highly pure material for chiral salt screening, INT 1f was recrystallized from CHCl₃ as an off-white solid (2:1 INT 1f/CHCl₃ solvate) in 95.1% recovery.

Batch 1—INT 1f. To a 4-neck 1 L round-bottom flask with overhead stirring, reflux condenser, addition funnel, thermocouple, and nitrogen inlet were charged malononitrile (13.55 g, 205.12 mmol, 2.08 equiv.) and 1,2-dimethoxyethane (sparged with nitrogen for 2 h, 495 mL, 15 vol). The solution was cooled to 0-5° C. with an ice-water bath and placed under nitrogen atmosphere. NaO Bu solution (2 M in THF, 0.71 wt % H₂O by KF titration, 98 mL, 196.86 mmol, 2.0 equiv.) was charged dropwise over 15 minutes, maintaining Tᵢₙₜₑᵣₙₐₗ≤10° C. The ice bath was removed, and the solution was allowed to stir at ambient temperature for 30 minutes. INT 1e (Purification—INT 1e, 34.39 g, 95.0 wt %=33.0 assay g, 98.43 mmol, 1.0 eq.) and Pd(dppf)Cl₂·CH₂Cl₂ (5.83 g, 7.14 mmol, 0.0725 eq) were charged to the reaction mixture in one portion. The headspace was flushed with nitrogen, and the reaction was refluxed (T=80-85° C.) overnight. At 16 h, the reaction was complete by HPLC analysis (1.5 area % INT 1e remaining, 87.9 area % INT 1f). The reaction was cooled to ambient temperature and filtered through a plug of silica gel (~66 g) with EtOAc (595 mL). The filtrate was extracted with water (3×100 mL), dried over Na₂SO₄ (~35 g), and filtered. The Na₂SO₄ was washed with EtOAc (165 mL). The organics were combined and concentrated under reduced pressure to low volume (~2 vols). The suspension was stirred at ambient temperature for 1 h then the solids were filtered and washed with cold EtOAc (50 mL) to give INT 1f as a light brown solid (27.86 g, 98.5 area %, 81.3 wt % by HPLC vs. working standard as the free base=22.65 assay g, 70.70 mmol) in 71.8% isolated yield.

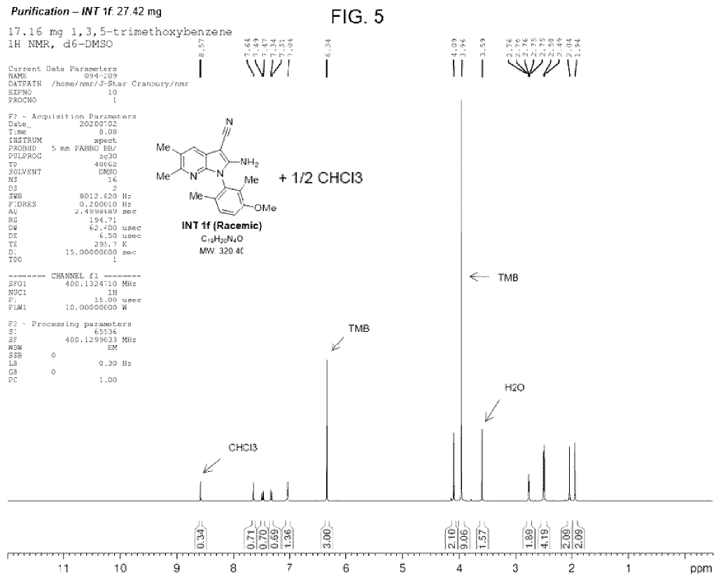

Purification—INT 1f: A suspension of INT 1f (Batch 1—INT 1f, 27.86 g, 98.5 area %, 81.3 wt % as the free base=22.65 assay g, 70.70 mmol) was suspended in CHCl₃ (113 mL) and stirred for 15 min then concentrated under reduced pressure to low volume (~1 vol). The slurry was then cooled in an ice bath for 1 h. The solid was filtered, washed with cold CHCl₃ (22 mL), and dried on the funnel overnight under a nitrogen atmosphere to afford INT 1f as an off-white solid (25.52 g, 99.3 area %, 84.6 wt % by 1H NMR vs. 1,3,5-trimethoxybenzene internal standard=21.58 assay g, 67.37 mmol; 100 wt % as 2:1 INT 1f/CHCl₃ solvate) in 95.1% recovery. Liquor and wash loss=4.6%. The ¹H NMR spectrum of the purified INT 1f is shown in FIG. 5.

### Example 6. Preparation Chiral Acid Salts of INT 1f, INT 1g, and Compound 1

INT 1f, INT 1g, and Compound 1 exhibit atropisomerism, and the desired enantiomer of Compound 1, has been isolated by chiral SFC, and assigned as the(S) enantiomer. Although resolution by chiral supercritical fluid chromatography (SFC) was amenable for isolating gram quantities of the desired enantiomer, it is inefficient for delivering kilograms of material. In order to overcome this, a chiral salt resolution for either INT 1f, INT 1g, or Compound 1 was considered. The salt resolution at INT 1f is likely a desired point of resolution.

For the resolution of INT 1f, 25 commercially available chiral acids (Scheme 6) were screened to determine whether salt formation would occur. For each of the acids, INT 1f (1.0 equiv.) and chiral acid (0.5 or 1.0 equiv.) were dissolved (DCM, ACN, or ACN/H₂O) and then concentrated to residue and analyzed by ¹H NMR (CDCl₃). For several acids screened, shifts of the ¹H NMR signals for the pyridine 4H proton (downfield 0.5 to 0.6 ppm) and methyl protons on the pyridine ring (downfield 0.2 to 0.3 ppm) were observed, suggesting the formation of a salt. In some cases, while no shifts in signals were observed, resolution of signals was obtained, suggesting salt formation as well. For the majority of chiral acids screened, no shift in signals or resolution were observed, suggesting that salt formation did not occur.

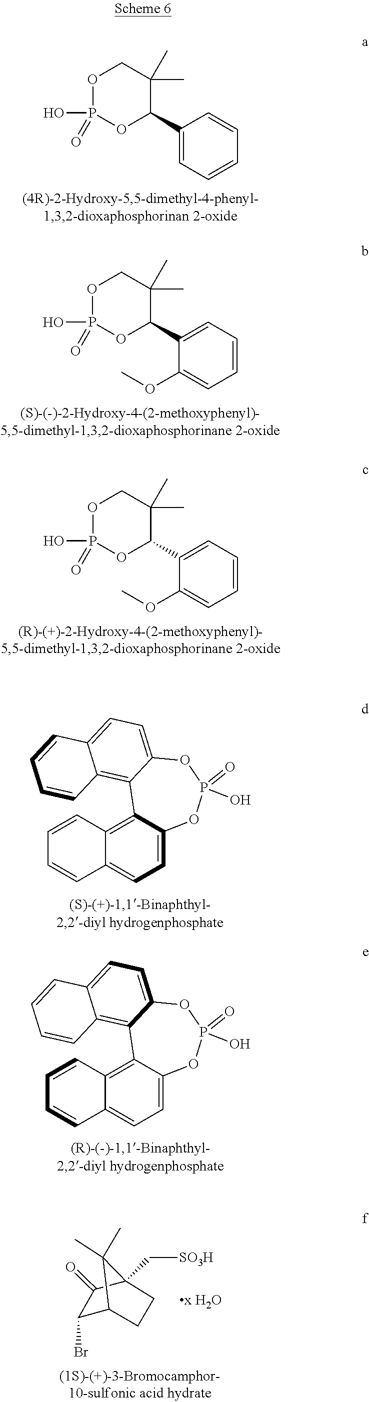

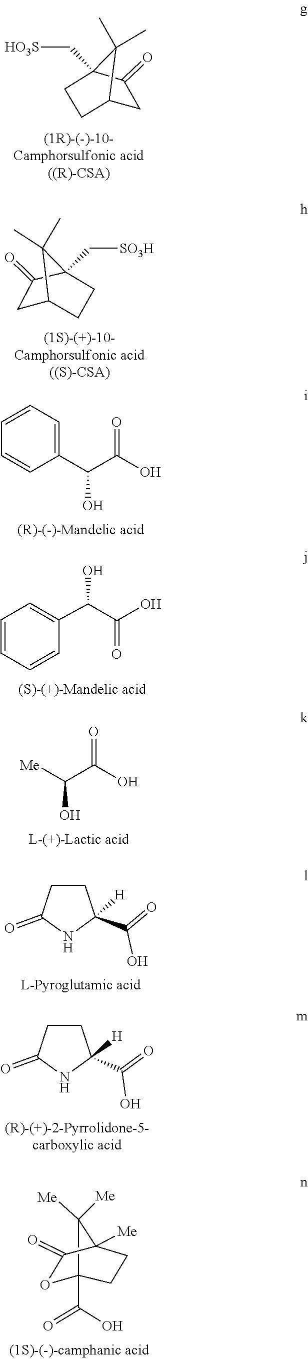

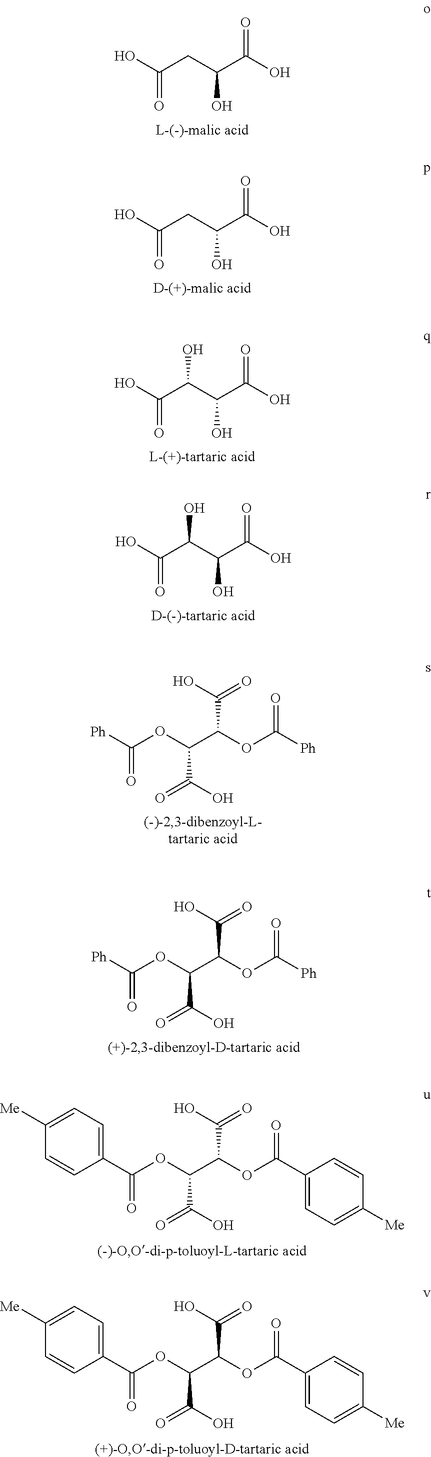

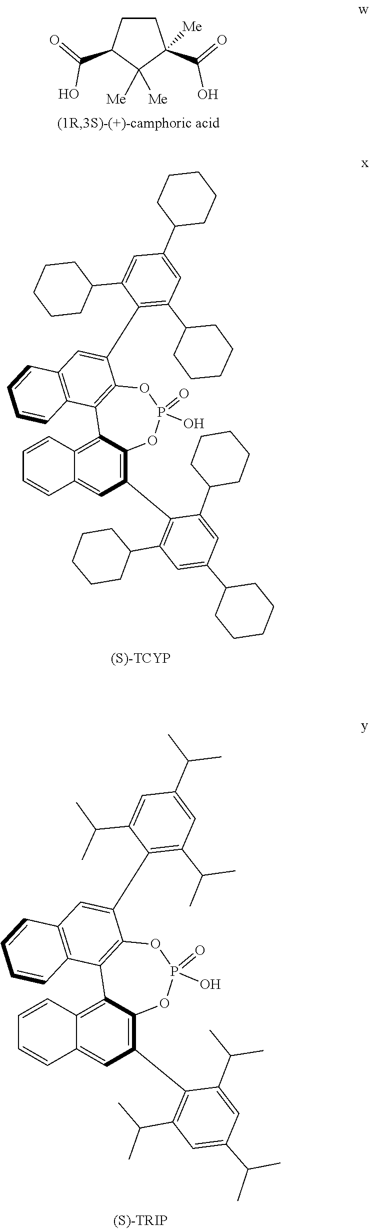

### Example 7. Crystallization of Chiral Acid Salts of INT 1f

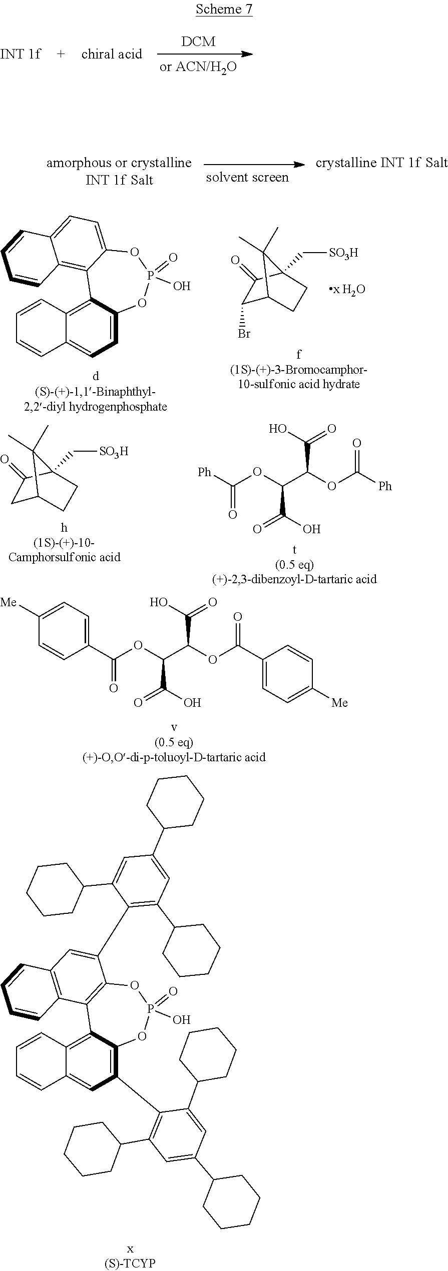

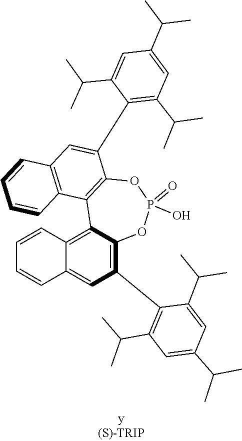

Salts formed from INT 1f with acids d, f, h, t, v, x, and y (Table 8) were submitted for crystallization screening.

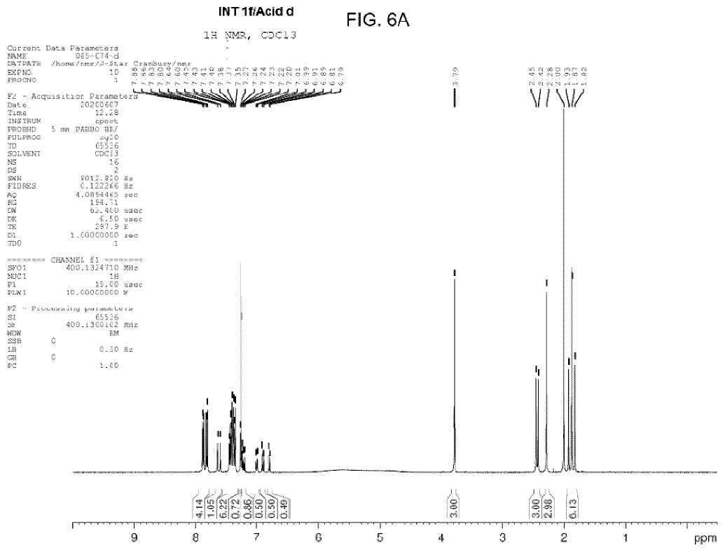

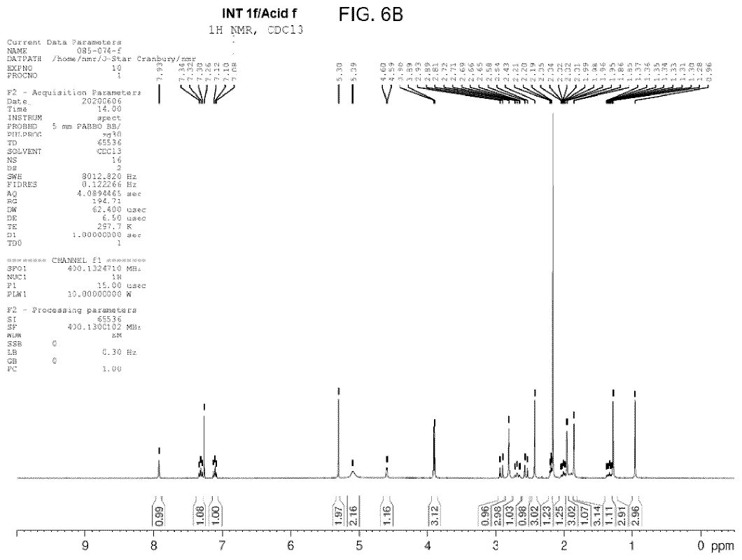

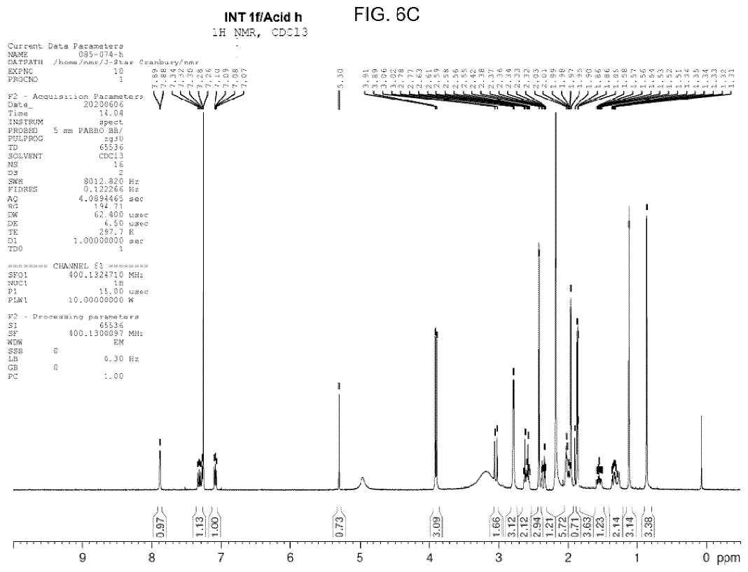

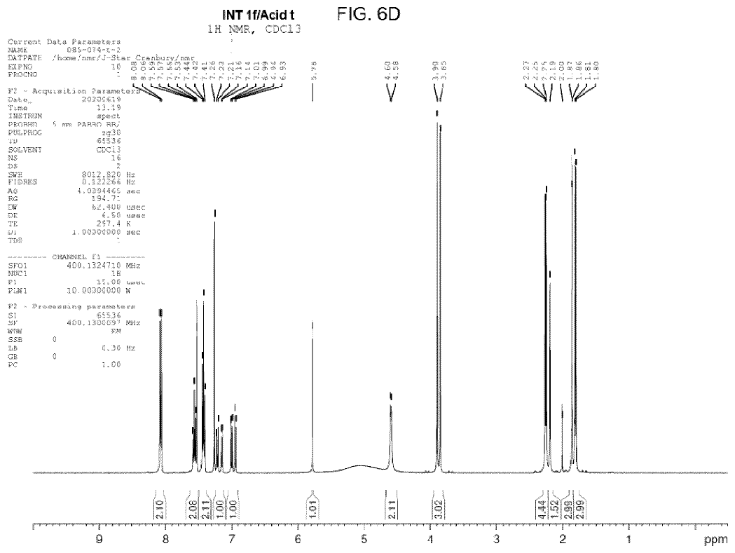

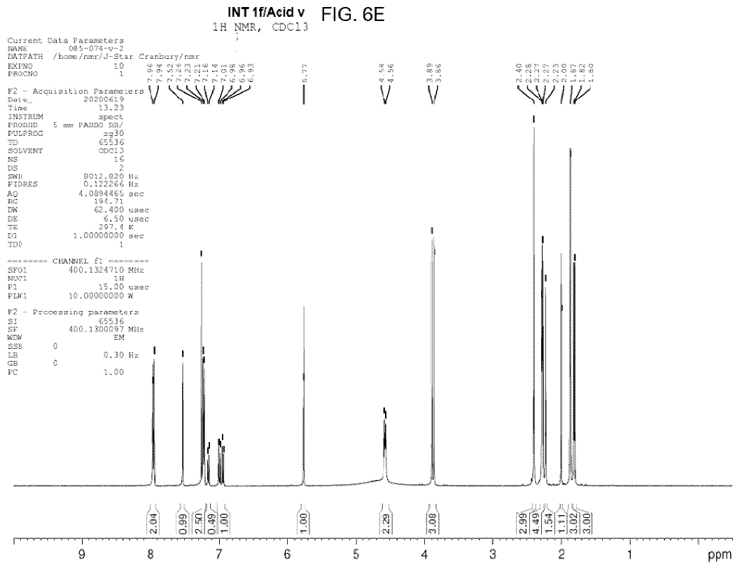

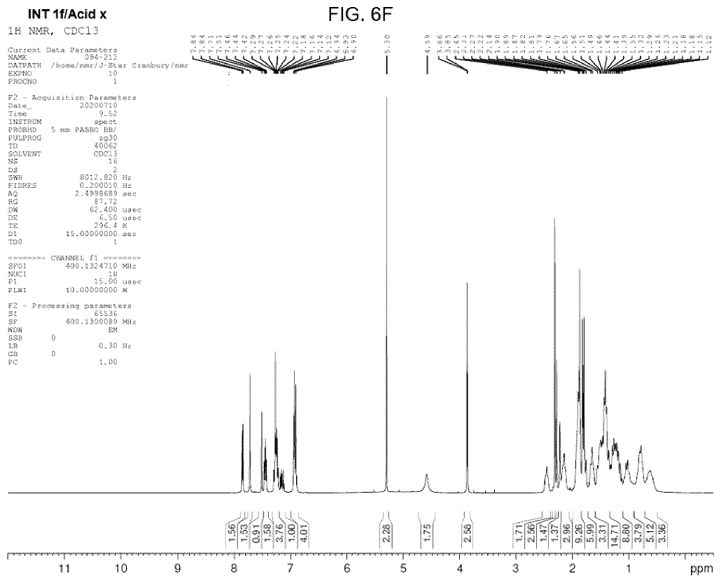

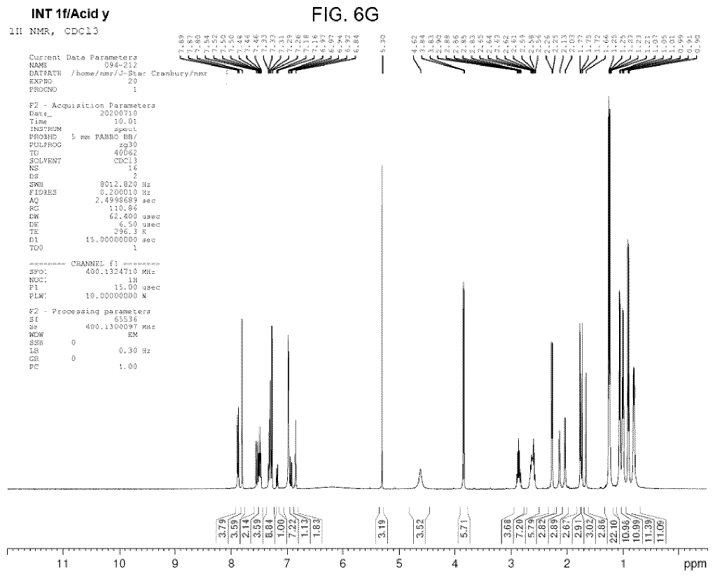

TABLE 8
Summary of INT 1f Salts Submitted for Solvent Screen

| Salt of | Chiral | Preparation of Chiral Acid | | |
|---|---|---|---|---|
| INT 1f | Acid | Salt of INT 1f | ¹H NMR | ¹H NMR Shifts |
| INT 1f/Acid d | d | Suspended in >80 vols ACN at | FIG. 6A | Protons resolving by |
| | | ambient temp, H₂O added dropwise | | NMR but no shift of |
| | | to obtain a solution. Concentrated to | | pyridine proton |
| | | residue. | | |
| INT 1f/Acid f | f | Dissolved in 10 vols DCM at | FIG. 6B | ¹H NMR Protons shifted |
| | | ambient temperature. Concentrated | | Salt |
| | | to residue. | | |
| INT 1f/Acid h | h | Dissolved in 10 vols DCM at | FIG. 6C | ¹H NMR Protons shifted |
| | | ambient temperature. Concentrated | | Salt |
| | | to residue. | | |
| INT 1f/Acid t | t | Suspended in >60 vols ACN at | FIG. 6D | Protons resolving by |
| | | ambient temp, H₂O added dropwise | | NMR but no shift of |
| | | to obtain a solution. Concentrated to | | pyridine proton |
| | | residue. | | |
| INT 1f/Acid v | v | Suspended in >60 vols ACN at | FIG. 6E | Protons resolving by |
| | | ambient temp, H₂O added dropwise | | NMR but no shift of |
| | | to obtain a solution. Concentrated to | | pyridine proton |
| | | residue. | | |
| INT 1f/Acid x | x | Dissolved in 10 vols DCM at | FIG. 6F | Protons resolving by |
| | | ambient temperature. Concentrated | | NMR but no shift of |
| | | to residue. | | pyridine proton |
| INT 1f/Acid y | y | Dissolved in 10 vols DCM at | FIG. 6G | Protons resolving by |
| | | ambient temperature. Concentrated | | NMR but no shift of |
| | | to residue. | | pyridine proton |

### Example 8. Crystallization of Chiral Acid Salts of INT 1g

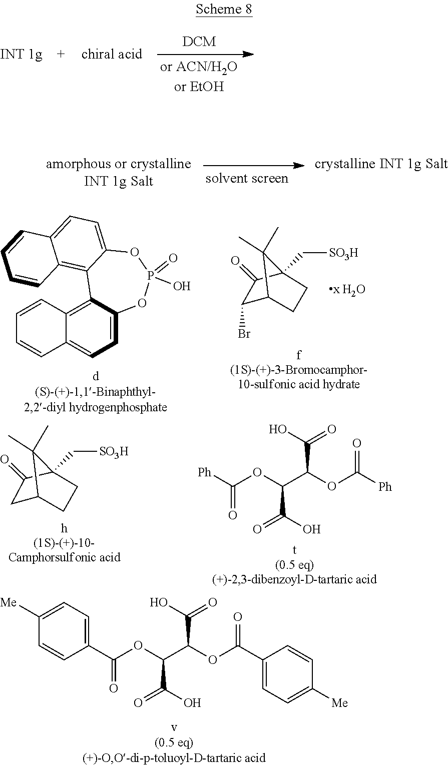

Salts formed with INT 1g and acids d, f, h, t, and v were submitted for crystallization screening (Table 9).

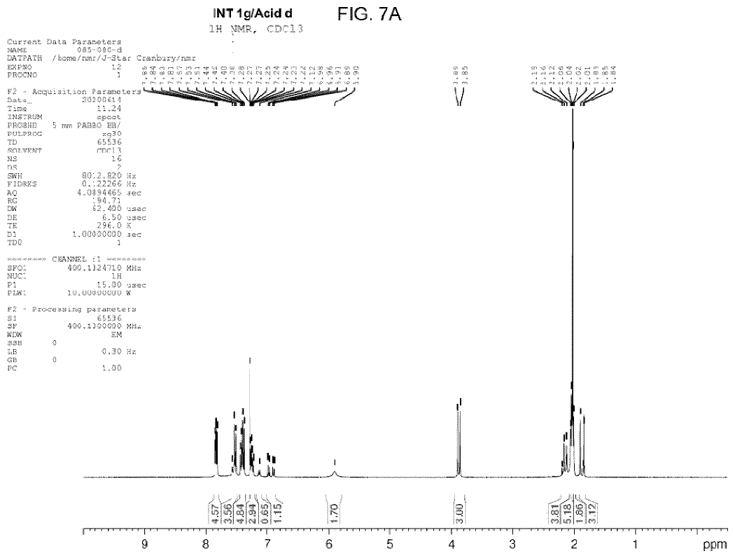

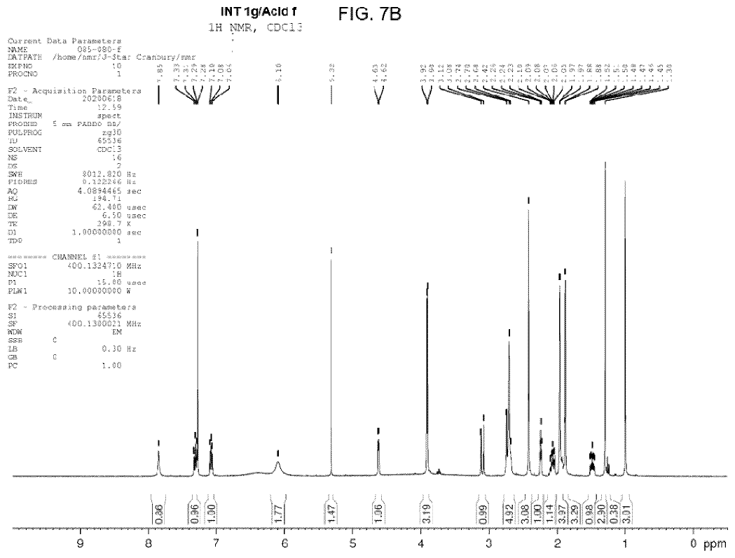

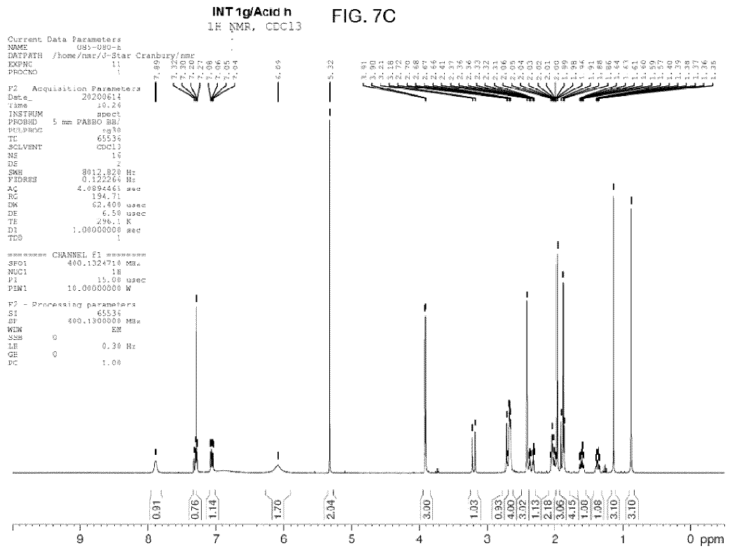

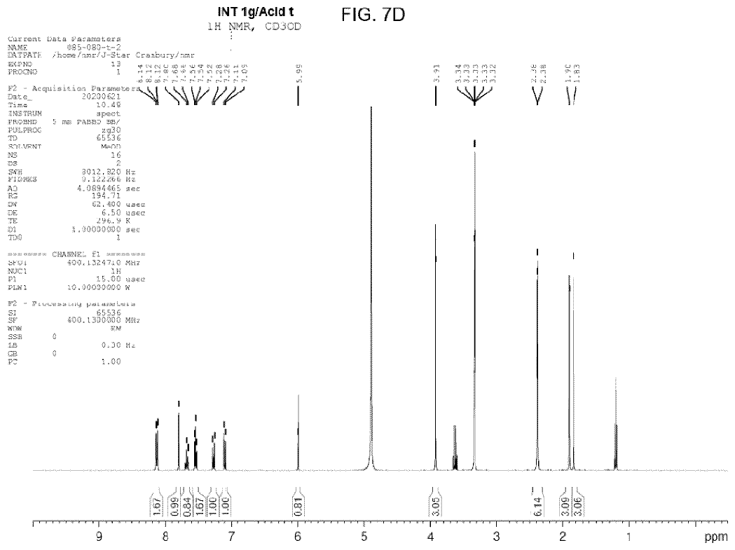

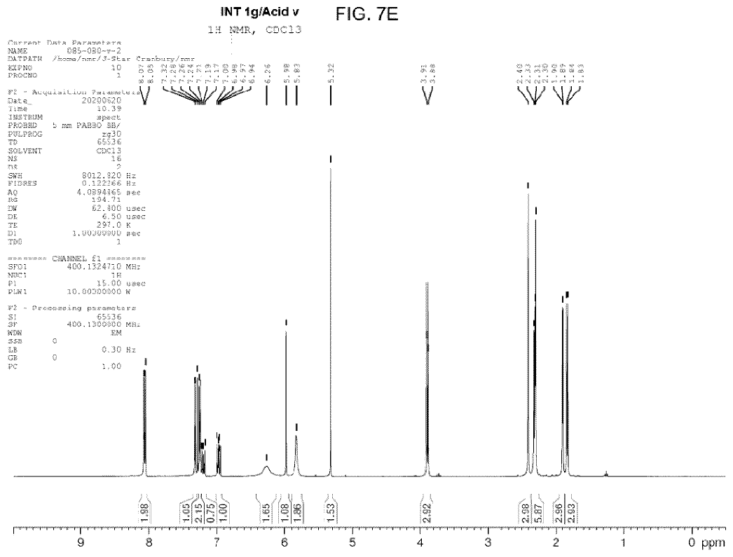

TABLE 9
Summary of INT 1g Salts Submitted for Solvent Screen

| | Chiral | Preparation of Chiral Acid | | |
|---|---|---|---|---|
| Salt of INT 1g | Acid | Salt of INT 1g | ¹H NMR | ¹H NMR Shifts |
| INT 1g/Acid d | d | Suspended in 10 vols ACN at 55° C., | FIG. 7A | Protons resolving by |
| | | H₂O added dropwise to obtain a | | NMR but no shift of |
| | | solution. Concentrated to residue. | | pyridine proton |
| INT 1g/Acid f | f | Dissolved in 2 vols DCM at ambient | FIG. 7B | ¹H NMR Protons shifted |
| | | temperature. Concentrated to residue. | | Salt |
| INT 1g/Acid h | h | Dissolved in 2 vols DCM at ambient | FIG. 7C | ¹H NMR Protons shifted |
| | | temperature. Concentrated to residue. | | Salt |
| INT 1g/Acid t | t | Dissolved in >60 vols EtOH at 70° C. | FIG. 7D | No peak shifts or |
| | | Concentrated to residue. | | resolution of peaks |
| | | | | compared to FB |
| INT 1g/Acid v | v | Dissolved in ~5 vols DCM at ambient | FIG. 7E | Protons resolving by |
| | | temperature. Concentrated to residue. | | NMR but no shift of |
| | | | | pyridine proton |

### Example 9. Crystallization of Chiral Acid Salts of Compound 1

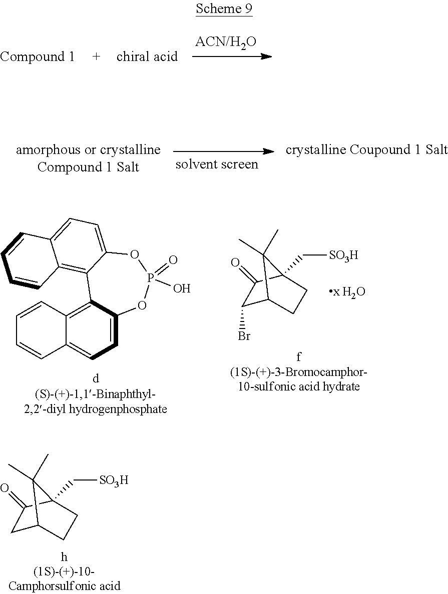

Salts formed with Compound 1 and acids d, f, and h were submitted for crystallization screening (Table 10).

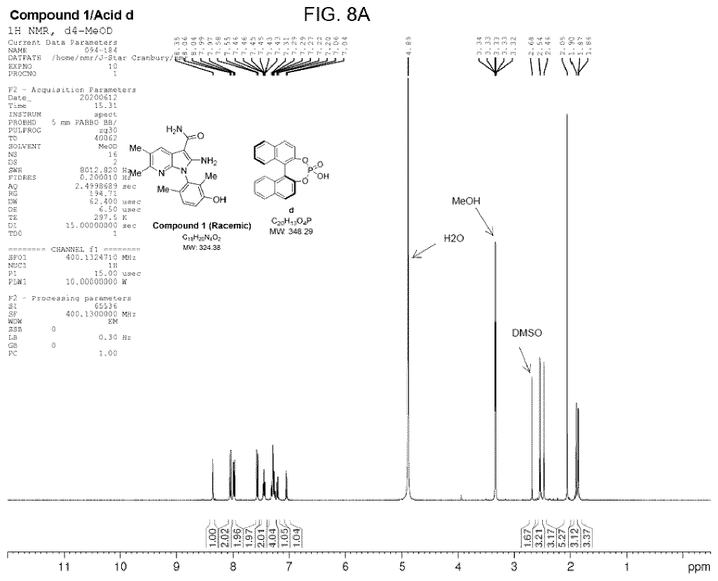

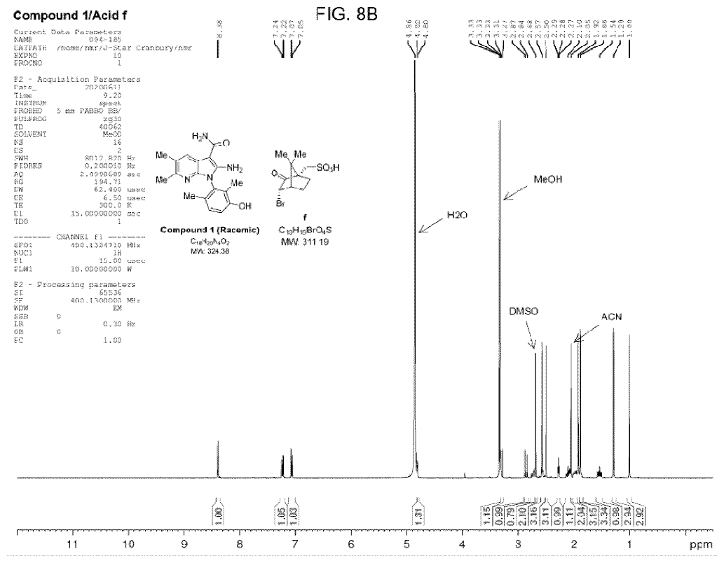

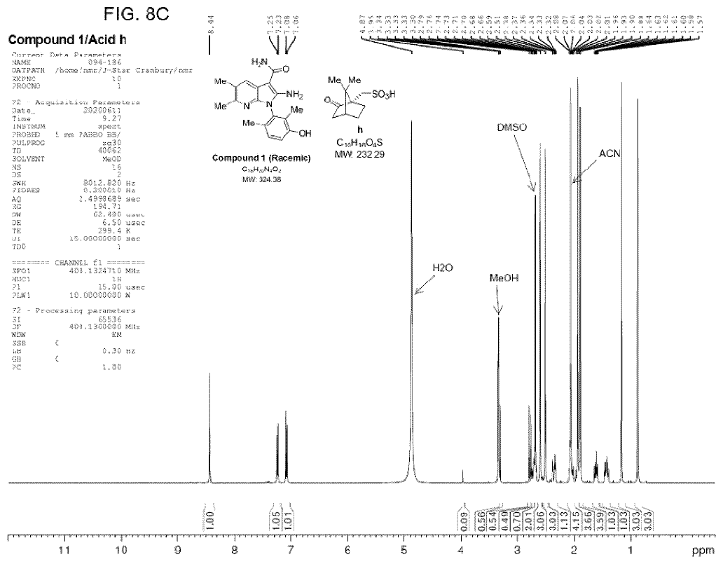

TABLE 10
Summary of Compound 1 Salts Submitted for Solvent Screen

| | Chiral | Preparation of Chiral Acid | | |
|---|---|---|---|---|
| Salt of Compound 1 | Acid | Salt of INT 1f | ¹H NMR | ¹H NMR Shifts |
| Compound 1/Acid d | d | Suspended in >40 vols ACN at 60- | FIG. 8A | ¹H NMR Protons |
| | | 70° C., 7.0 mL H₂O added dropwise | | shifted |
| | | to obtain a solution. Concentrated | | Salt |
| | | to residue. | | |
| Compound 1/Acid f | f | Suspended in >40 vols ACN at 60- | FIG. 8B | ¹H NMR Protons |
| | | 70° C., 3.0 mL H₂O added dropwise | | shifted |
| | | to obtain a solution. Concentrated | | Salt |
| | | to residue. | | |
| Compound 1/Acid h | h | Suspended in >40 vols ACN at 60- | FIG. 8C | ¹H NMR Protons |
| | | 70° C., 4.0 mL H₂O added dropwise | | shifted |
| | | to obtain a solution. Concentrated | | Salt |
| | | to residue. | | |

### Example 10. INT 1f Chiral Salt Crystallizations

INT 1f salts (50-60 mg) were weighed into 4 ml vials. Solvent (~0.2-1.0 mL) was added to each vial, and the vials were stirred at ambient temperature for 3 days. Additional solvent was added to vials with thick slurries, and additional salts were added to vials with clear solutions. The results are summarized in the following Tables 11-17.

No enantioenrichment was observed for salts with acids d (Table 11), f (Table 12), t (Table 14), v (Table 15), x (Table 16), or y (Table 17).

TABLE 11
INT 1f/(S)-(+)-1,1′-Binaphthyl-2,2′-diyl Hydrogen Phosphate Salt Screen (Acid d)

| | | Enantiomer Ratio | Approximate Solubility of |
|---|---|---|---|
| Entry | Solvent | HPLC (precipitated solids) | Starting Material |
| 1 | EtOH | 50:50 | Very soluble |
| 2 | EtOAc | 51:49 | Soluble |
| 3 | MeCN | 49:51 | Soluble |
| 4 | THF | — | Highly soluble |
| 5 | DCM | — | Highly soluble |
| 6 | 1,4-Dioxane | — | Highly soluble |
| 7 | Toluene | 50:50 | Very soluble |
| 8 | Acetone:water (9:1) | 50:50 | Very soluble |
| 9 | EtOH:Heptane (1:1) | 50:50 | Soluble |
| 10 | Anisole | 50:50 | Very soluble |

—: No solids afforded for LC analysis even after additional material was added to the
samples

TABLE 12
INT 1f/(1S)-(+)-3-Bromocamphor-10-sulfonic Acid Hydrate Salt Screen (Acid f)

| | | Enantiomer Ratio | Approximate Solubility of |
|---|---|---|---|
| Entry | Solvents | HPLC (solids) | Starting Material |
| 1 | EtOH | 50:50 | Very soluble |
| 2 | EtOAc | 50:50 | Very soluble |
| 3 | MeCN | — | Highly soluble |
| 4 | THF | — | Highly soluble |
| 5 | DCM | — | Highly soluble |
| 6 | 1,4-Dioxane | — | Highly soluble |
| 7 | Toluene | 50:50 | Soluble |
| 8 | Acetone:water (9:1) | — | Highly soluble |
| 9 | EtOH: Heptane (1:1) | — | Highly soluble |
| 10 | Anisole | 50:50 | Soluble |

—: No solids afforded for LC analysis even after more material was added to the samples

TABLE 13
INT 1f/(S)-CSA Salt Screen (Acid h)

| | | Enantiomer Ratio | Approximate Solubility of |
|---|---|---|---|
| Entry | Solvents | HPLC (solids) | Starting Material |
| 1 | EtOH | 50:50 | Very soluble |
| 2 | EtOAc | 50:50 | Soluble |
| 3 | MeCN | — | Highly soluble |
| 4 | THF | 50:50 | Very soluble |
| 5 | DCM | 50:50 | Very soluble |
| 6 | 1,4-Dioxane | 50:50 | Soluble |
| 7 | Toluene | — | Highly soluble |
| 8 | Acetone:water (9:1) | — | Highly soluble |
| 9 | EtOH: Heptane (1:1) | 50:50 | Soluble |
| 10 | Anisole | 15:85 | Very soluble |
| | | 81:19 (ML) | |

—: No solids afforded for LC analysis even after additional material was added to the samples

TABLE 14
INT 1f/(+)-2,3-Dibenzoyl-D-tartaric Acid Salt Screen (Acid t)

| | | Enantiomer Ratio | Approximate Solubility of |
|---|---|---|---|
| Entry | Solvents | HPLC (solids) | Starting Material |
| 1 | EtOH | 50:50 | Soluble |
| 2 | EtOAc | 50:50 | Soluble |
| 3 | MeCN | 50:50 | Soluble |
| 4 | THF | — | Highly soluble |
| 5 | DCM | — | Highly soluble |
| 6 | 1,4-Dioxane | — | Highly soluble |
| 7 | Toluene | 50:50 | Soluble |
| 8 | Acetone:water (9:1) | 50:50 | Soluble |
| 9 | EtOH: Heptane (1:1) | 50:50 | Soluble |
| 10 | Anisole | 50:50 | Soluble |

—: No solids afforded for LC analysis even after additional material was added to the samples
Note:
Salt consisted of 2:1 ratio INT 1f:Acid t.

TABLE 15
INT 1f/(+)-O,O′-Di-p-toluoyl-D-tartaric Acid Salt Screen (Acid v)

| | | Enantiomer Ratio | Approximate Solubility of |
|---|---|---|---|
| Entry | Solvents | HPLC (solids) | Starting Material |
| 1 | EtOH | 50:50 | Soluble |
| 2 | EtOAc | 50:50 | Soluble |
| 3 | MeCN | 50:50 | Soluble |
| 4 | THF | 50:50 | Very soluble |
| 5 | DCM | 50:50 | Very soluble |
| 6 | 1,4-Dioxane | 50:50 | Very soluble |
| 7 | Toluene | 37:62 (1^{st} run) | Soluble |
| | | 39:61 (2^{nd} run) | |
| | | 79:21 (ML) | |
| 8 | Acetone:water (9:1) | 50:50 | Soluble |
| 9 | EtOH: Heptane (1:1) | 50:50 | Soluble |
| 10 | Anisole | 50:50 | Soluble |

Note:
Salt consisted of 2:1 ratio INT 1f:Acid v.

TABLE 16
INT 1f/(S)-TCYP Salt Screen (Acid x)

| | | Enantiomer Ratio | Approximate Solubility of |
|---|---|---|---|
| Entry | Solvents | HPLC (solids) | Starting Material |
| 1 | EtOH | 50:50 | Very soluble |
| 2 | EtOAc | — | Highly soluble |
| 3 | MeCN | 53:47 (1^{st} run) | Very soluble |
| | | 51:49 (2^{nd} run) | |
| 4 | THF | — | Highly soluble |
| 5 | DCM | — | Highly soluble |
| 6 | 1,4-Dioxane | 51:49 (1^{st} run)/ | Soluble |
| | | 49:51 (2^{nd} run) | |
| 7 | Toluene | 50:50 | Very soluble |
| 8 | Acetone:water (9:1) | 50:50 | Soluble |
| 9 | EtOH: Heptane (1:1) | 50:50 | Soluble |
| 10 | Anisole | 51:49 | Very soluble |

—: No solids afforded for LC analysis even after additional material was added to the samples

TABLE 17
INT 1f/(S)-TRIP Salt Screen (Acid y)

| | | Enantiomer Ratio | Approximate Solubility of |
|---|---|---|---|
| Entry | Solvents | HPLC (solids) | Starting Material |
| 1 | EtOH | 50:50 | Soluble |
| 2 | EtOAc | 50:50 | Very soluble |
| 3 | MeCN | 50:50 | Soluble |
| 4 | THF | — | Highly soluble |
| 5 | DCM | 50:50 | Highly soluble |
| 6 | 1,4-Dioxane | — | Highly soluble |
| 7 | Toluene | 50:50 | Highly soluble |
| 8 | Acetone:water (9:1) | 50:50 | Highly soluble |
| 9 | EtOH: Heptane (1:1) | — | Soluble |
| 10 | Anisole | 51:49 | Very soluble |

—: No solids afforded for LC analysis even after additional material was added to the samples

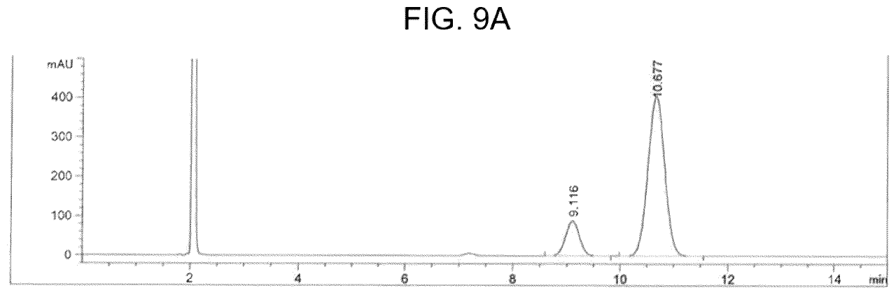

Entry 10 of Table 13, (anisole solvent, acid h ((S)-CSA) demonstrated a very good chiral purity in the recovered solids of 15:85 (see FIG. 9A). The chiral purity of the mother liquor was determined to be 81:19 (FIG. 9B). This suggests that the use of the enantiomer of acid h (i.e., acid g; (R)-CSA) in the methods described above would produce an enantioenriched solid of (S)-INT 1f/(R)-CSA).

### Example 11. INT 1f Chiral Salt Crystallizations-Aromatic Solvent Screen

Following the encouraging result employing anisole as the solvent and switching to (R)-CSA to afford the desired diastereomer, the following common aromatic solvents were screened: 1,3-dimethoxybenzene;

2-, 3-, and 4-methyanisoles; o, m, and p-xylenes; and α,α,α-trifluorotoluene (Table 18). Among the nine solvent systems, INT 1f/(R)-CSA salt showed high solubility in 1,3-dimethoxybenzene and anisole, and good solubility in 2-, 3-, 4-methyanisole with heating, but poor solubility in other solvents. In addition to anisole, 4-methylanisole and 1,3-dimethoxybenzene also afforded enantioenriched crystalline solids with enantiomeric ratios (e.r.) of 90:10 and 87:13, respectively. However, the highest e.r. was obtained with anisole (92:8).

TABLE 18
Crystallization of INT 1f/(R)-CSA Salt in Aromatic Solvents

| | Salt | | Vol | | |
|---|---|---|---|---|---|
| Entry | (mg) | Solvent | (mL) | Observations | e.r.* |
| 1 | 147 | anisole | 0.4 | 90 mg in 0.4 mL, clear; RT, overnight, | 85:15 |
| | | | | clear; added 57 mg, clear; overnight, | |
| | | | | slurry | |
| 1a | 100 | anisole | 2 | Add 1 mL, clear solution; light seeding, | 92:8 |
| | | | | thick slurry; add 1 mL, slurry; 80° C., | |
| | | | | clear; crystallization at ~45° C.; 40° C. | |
| | | | | overnight to RT | |
| 2 | 200 | anisole | 4 | Clear solution; light seeding; | 89:11 |
| | | | | crystallization at ~45° C.; 40° C. overnight | |
| | | | | to RT | |
| 3 | 100 | 2-methylanisole | 4 | Thick slurry; add 1 mL; add 2 mL; 80° C., | 50:50 |
| | | | | clear; crystallization at ~45° C.; 40° C. | |
| | | | | overnight to RT | |
| 4 | 100 | 3-methylanisole | 4 | Thin slurry + oil; add 0.5 mL; add 2 mL, | 50:50 |
| | | | | cloudy; 80° C., clear; slight cloudy at | |
| | | | | ~45° C.; 40° C. overnight, clear; light | |
| | | | | seeding at RT; add 50 mg RT | |
| 5 | 100 | 4-methylanisole | 4 | Thick slurry; add 1 mL; add 2 mL; 80° C., | 90:10 |
| | | | | clear; crystallization at ~45° C.; 40° C. | |
| | | | | overnight to RT | |
| 6 | 100 | m-xylene | 4 | Thick slurry; add 1 mL; add 2 mL; 80° C., | 52:48 |
| | | | | less thick slurry; 40° C. overnight, slurry; | |
| | | | | RT | |
| 7 | 100 | o-xylene | 4 | Thick slurry; add 1 mL; add 2 mL; 80° C., | 57:43 |
| | | | | less thick slurry; 40° C. overnight, slurry + | |
| | | | | oil; RT | |
| 8 | 100 | p-xylene | 4 | Thick slurry; add 1 mL; add 2 mL; 80° C., | 50:50 |
| | | | | less thick slurry; 40° C. overnight to RT | |
| 9 | 200 | 1,3- | 1 | Clear solution + seeding, still clear; add | 87:13 |
| | | dimethoxybenzene | | 50 mg; RT overnight, very thin slurry; | (thin slurry) |
| | | | | add 40 mg; add 10 mg, near clear with | 85:15 |
| | | | | heating; RT overnight, slurry | (thick |
| | | | | | slurry) |
| 10 | 100 | α,α,α- | 4 | Thick slurry; add 1 mL; add 2 mL; 80° C., | 53:47 |
| | | trifluorotoluene | | less thick slurry; 40° C. overnight, thick | |
| | | | | slurry; RT | |

*Enantiomer Ratio HPLC (solids)

The solids obtained from anisole, 4-methylanisole and 1,3-dimethoxybenzene were slurried at 40-45° C. over the weekend to look at the impact on e.r. (Table 19). Anisole showed the highest e.r. upgrade with an increase from 92:8 to 95:5. Anisole was chosen as a solvent for further development.

TABLE 19
Aging of INT 1f/(R)-CSA Salt Slurries

| Entry No. | | | | | |
|---|---|---|---|---|---|
| from Table | Salt | | Volume | e.r. from Table | e.r. after aged at |
| 18 | (mg) | Solvent | (mL) | 18 | 40-45° C. for 3 days |
| 1a | 100 | anisole | 2 | 92:8 | 95:5 |
| 2 | 200 | anisole | 4 | 89:11 | 93:7 |
| 5 | 100 | 4-methylanisole | 4 | 90:10 | 93:7 |
| 9 | 200 | 1,3- | 3* | 87:13, 85:15 | 93:7 |
| | | dimethoxybenzene | | | |

* Solvent volume was 1 mL before aging. Additional 2 mL added.

### Example 12. INT 1f Chiral Salt Resolution Process Development-Synthesis of (S)-Compound 1 from(S)-INT 1f/(R)-CSA

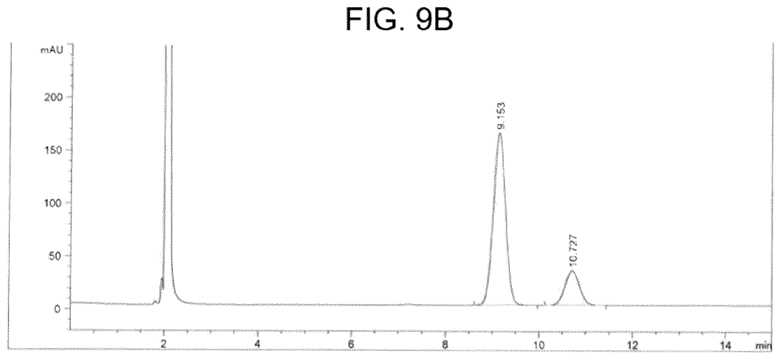

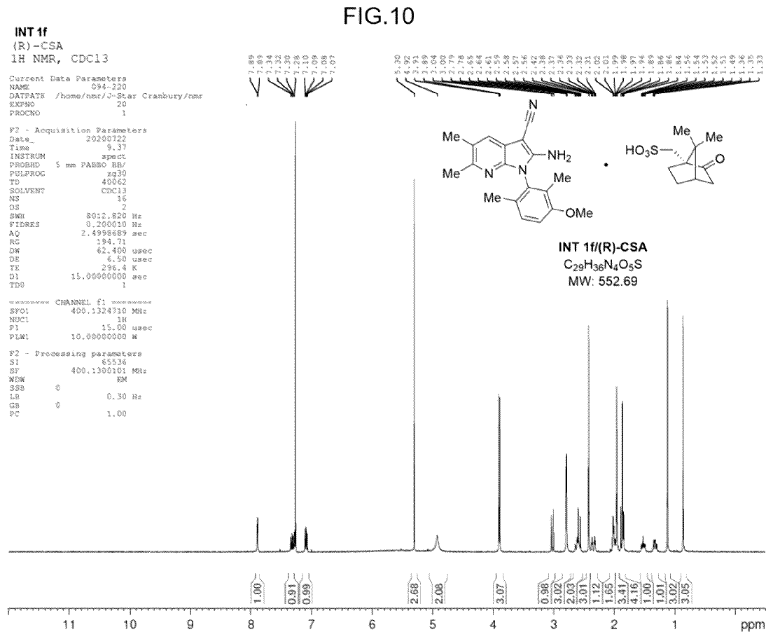

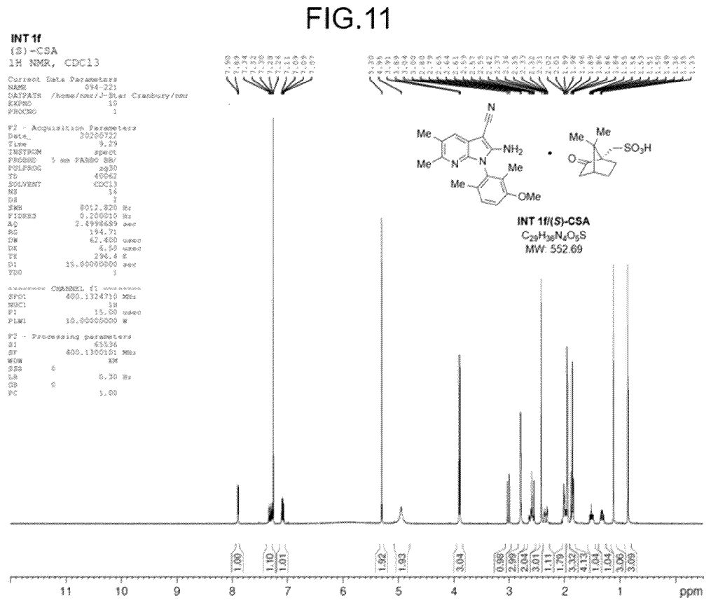

Initial development work was centered around the(S)-CSA salt of INT 1f. To determine which enantiomer of camphorsulfonic acid (CSA) afforded(S)-Compound 1, the (R)- and(S)-camphorsulfonic acid salts of INT 1f were prepared by concentrating a 1:1 solution of INT 1f and chiral acid to residue to afford ~6 g of each salt. Salt formation was determined by the pyridine proton chemical shift and proton resolution in the ¹H NMR spectra. The ¹H NMR spectrum of INT 1f/(R)-CSA is shown in FIG. 10. The 1H NMR spectrum of INT 1f/(S)-CSA is shown in FIG. 11.

Both salts were enriched by crystallization from anisole (40 vols) at 45° C. for about 16-18 h. Each racemic salt was enriched to ~9:1 e.r. and was then converted to the corresponding atropisomers of Compound 1. It was found that the salt derived from (1S)-(+)-10-camphorsulfonic acid afforded (R)-Compound 1, whereas the salt derived from (1R)-(−)-10-camphorsulfonic acid gave desired (S)-Compound 1 (Scheme 10).

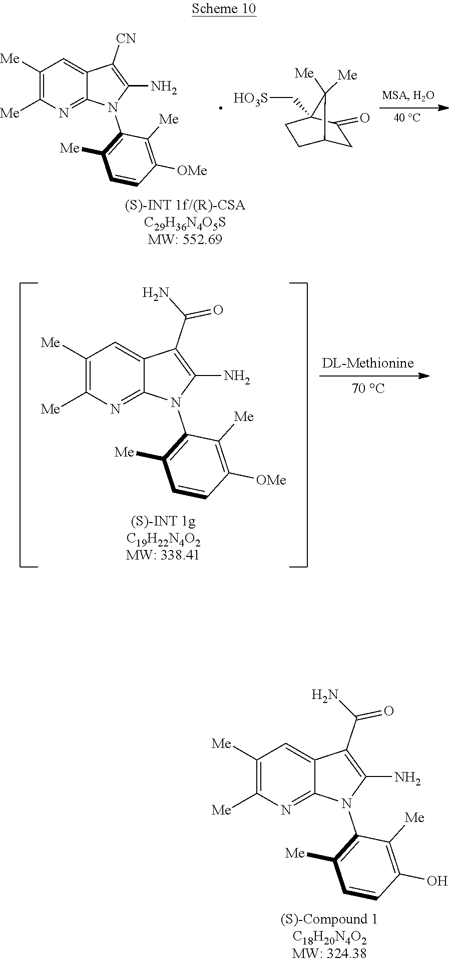

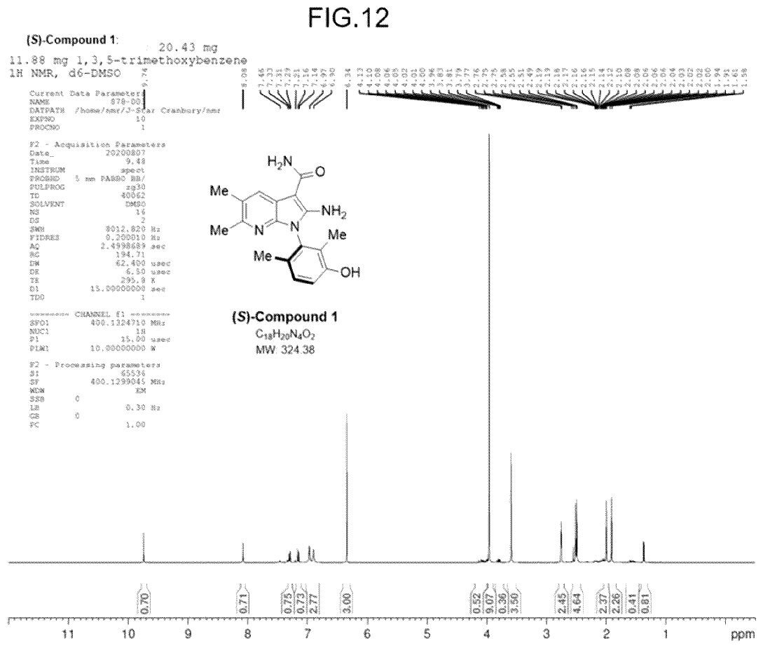

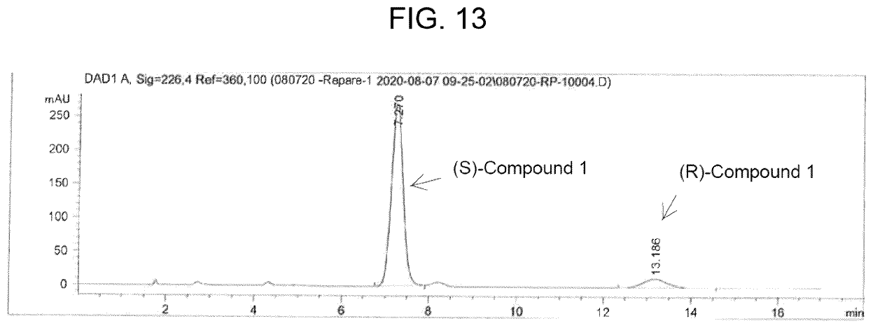

Batch 1—(S)-INT 1f/(R)-CSA: (S)-INT 1f/(R)-CSA (0.37 g, 99.1 area %, 82.5 wt %=0.31 assay g, 0.55 mmol, 91.0:9.0 er) was carried into the one-pot synthesis of (S)-Compound 1. Heating with methanesulfonic acid (1.43 mL, 40.0 eq) and H₂O (30.5 μL, 4.0 eq) at 40° C. for 1 h afforded almost complete conversion to(S)-INT 1g (5.8 area % (S)-INT 1f, Table 20). The solution was cooled to ambient temperature and DL-methionine (0.33 g, 4.0 eq) was charged. After heating at 70-72° C. overnight, the reaction was nearly complete (3.0 area % (S)-INT 1g, 86.6 area % (S)-Compound 1). The reaction was quenched with aq. ammonia (28 wt %) to pH=9. The solids were isolated by filtration and dried under vacuum overnight to afford a yellow solid (0.177 g, 93.6 area %, 79.6 wt % by ¹H NMR vs. 1,3,5-trimethoxybenzene internal standard=0.141 assay g, 0.43 mmol) in 79.2% isolated yield with 91.1:8.9 er. This confirms that(S)-INT 1f/(R)-CSA affords(S)-Compound 1 with no chiral erosion (Table 20). The 1H NMR spectrum of (S)-Compound 1 is shown in FIG. 12. The chiral HPLC of (S)-Compound 1 produced by this method is shown in FIG. 13.

TABLE 20
Synthesis of (S)-Compound 1

| | | LCAP | | |
|---|---|---|---|---|
| Entry | Description | INT 1f | INT 1g | Compound 1 |
| 1 | IPC: Step 1 - 1 h | 5.8 | 88.6 | N/A |
| 2 | IPC: Step 2 - overnight | nd¹ | 3.0 | 86.6 |
| 3 | Dried solid: | nd¹ | 2.2 | 93.6 |
| | 91.1:8.9 er | | | |

¹nd = not detected

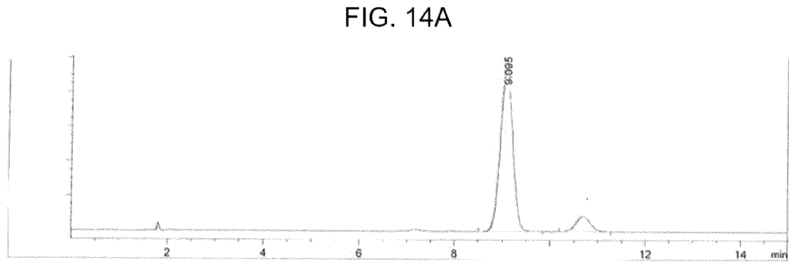

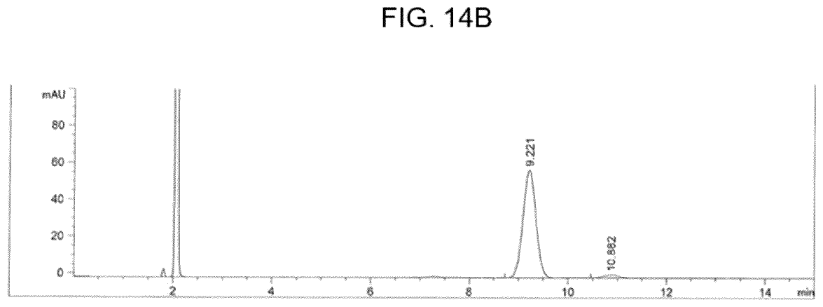

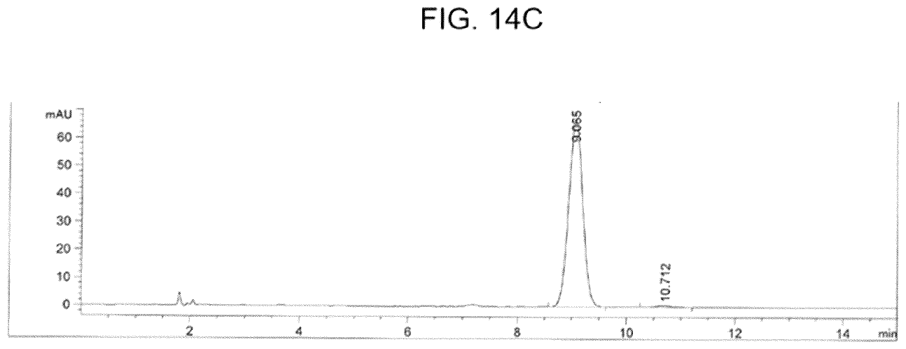

After identifying which enantiomer of camphorsulfonic acid was needed, we then looked at how to develop a process that would deliver enantiomerically enriched(S)-INT 1f. Enantioenriched(S)-INT 1f/(R)-CSA could be crystallized from either the pre-made racemic salt or prepared in situ from racemic INT 1f and (1R)-(−)-10-camphorsulfonic acid via a reactive crystallization. With both methods, the initial crystallization (40 vols anisole 40-45° C., ~16 h) affords ~90:10 enantiomeric ratio (e.r.). Once isolated, additional rounds of recrystallization of (S)-INT 1f/(R)-CSA lead to enrichment from 90:10 e.r to 97:3 e.r. (2^{nd} round) to 99:1 e.r. (3^{rd} round, Table 21). The chiral HPLC spectra associated with each entry in Table 21 are provided in FIGS. 14A-14C.

TABLE 21
(S)-INT 1f/(R)-CSA e.r. Enrichment via Recrystallization Cycles

| | Initial | Equil. | LCAP | | Recovery |
|---|---|---|---|---|---|
| Description | e.r. | time (h) | Peak 1 | Peak 2 | (%) |
| Crystallization 1 - (S)-INT 1f/(R)-CSA | racemic | 3 days | 90.3 | 9.7 | 51.4 |
| Crystallization 2 - (S)-INT 1f/(R)-CSA | 90.0:10.0 | 7 | 96.8 | 3.2 | 96.1 |
| (2^{nd} round, wet cake) | | | | | |
| Crystallization 2 - (S)-INT 1f/(R)-CSA | 96.8:3.2 | 4 | 99.0 | 1.0 | 29.8 |
| (3^{rd} round, dried solid) | | | | | |

When the enantioenriched salt was not isolated after the first crystallization, and instead, submitted to temperature cycling to enhance the e.r., little improvement in the e.r. was observed regardless of the amount of solvent used (Table 22). Regarding isolation of (S)-INT 1f/(R)-CSA, cooling the recrystallization prior to filtration greatly improved recovery from 51.0% to 89.6%.

TABLE 22
Initial Crystallization of (S)-INT 1f/(R)-CSA

| | | LCAP | | |
|---|---|---|---|---|
| Description | | Peak 1 | Peak 2 | Isolated Yield (%) |
| Crystallization 3 - (S)-INT 1f/(R)-CSA | Cycle 1 | 86.8 | 13.2 | 51.0 |
| (salt formation in situ) | Cycle 2 | 90.0 | 10.0 | (hot filtered) |
| 50 vols anisole | Cycle 3 | 91.0 | 9.0 | |
| Crystallization 4 - (S)-INT 1f/(R)-CSA | Cycle 1 | 89.0 | 11.0 | 89.6 |
| (salt formation in situ) | | | | |
| 20 vols anisole¹ | Cycle 2 | 86.8 | 13.2 | (20-23° C. filtered) |

¹For mass of salt formed

Of the possible approaches mentioned above, it appeared that initial crystallization followed by recrystallization from anisole would be an apt process to develop further. Consequently, a process whereby enriched(S)-INT 1f is formed by a reactive crystallization followed by recrystallization to give(S)-INT 1f in ~ 95:5 e.r. was investigated.

### Example 13. Two Step Crystallization Process and Final Process Development

For the following development work, the yield is expressed as recovery of available chiral INT 1f free base. For example, in Reactive Crystallization-(S)-INT 1f/(R)-CSA, the corrected total INT 1f intake is 5.00 g, but recovery is based on 2.50 g INT 1f with the correct configuration.

Step 1. Reactive Crystallization—(S)-INT 1f/(R)-CSA

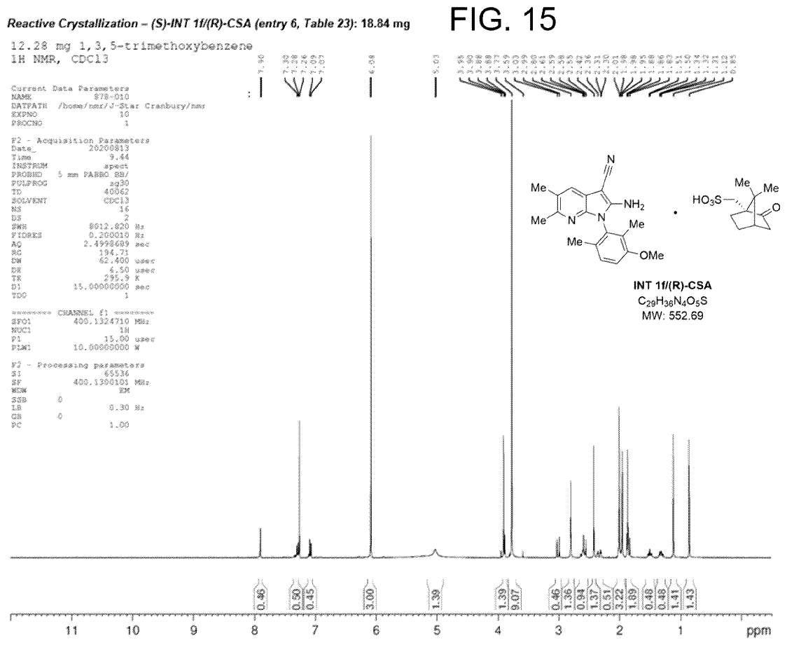

Reactive crystallization of INT 1f (Purification—INT 1f, 5.91 g, 99.3 area %, 84.6 wt %=5.00 g, racemic) with (1R)-(−)-10-camphorsulfonic acid (1.0 equiv.) was conducted in anisole (34.5 vol of free base, 20 vol of salt) to determine whether 99:1 e.r. seed could improve the enantioselectivity during the first crystallization. Neither equilibration at ambient temperature (Table 23, entry 2) nor at 45-50° C. (entry 4) after seeding improved the e.r. above that typically observed. Cooling to 0-10° C. prior to isolation by filtration did not erode e.r., but reduced liquor losses from 17 to 7 mg/mL. (S)-INT 1f/(R)-CSA was isolated by filtration and dried in a vacuum oven at 50° C. overnight to give salt (3.73 g, 99.1 area %, 98.6 wt %=3.68 assay g) with a trace of anisole and with 85.3:14.7 e.r. in 74% recovery. The ¹H NMR spectrum of (S)-INT 1f/(R)-CSA (entry 6, Table 23) is shown in FIG. 15.

TABLE 23
Reactive Crystallization - (S)-INT 1f/(R)-CSA

| Entry | Description | | e.r. |
|---|---|---|---|
| 1 | Cycle 1 | IPC | 82.8:17.2 |
| | | 1. Age: 45-50° C. for 30 min | |
| | | 2. Seed (99:1 e.r.) | |
| | | 3. Age: rt for 4 h | |
| 2 | | IPC | 83.5:16.5 |
| | | Age: rt overnight | |
| 3 | Cycle 2 | IPC | 86.4:13.6 |
| | | 1. Age: 75-80° C. for 30 min | |
| | | 2. Seed (99:1 er) | |
| | | 3. Age: 45-50° C. for 4 h | |
| 4 | | IPC | 87.6:12.4 |
| | | Age: 45-50° C. overnight | |
| 5 | Isolation | Mother liquor | 20.3:79.7 |
| 6 | | Solid (3.73 g of salt) | 85.3:14.7 |
| | | 99.1 area % | |
| | | 98.6 wt % | |

Step 2. Recrystallization 1—(S)-INT 1f/(R)-CSA

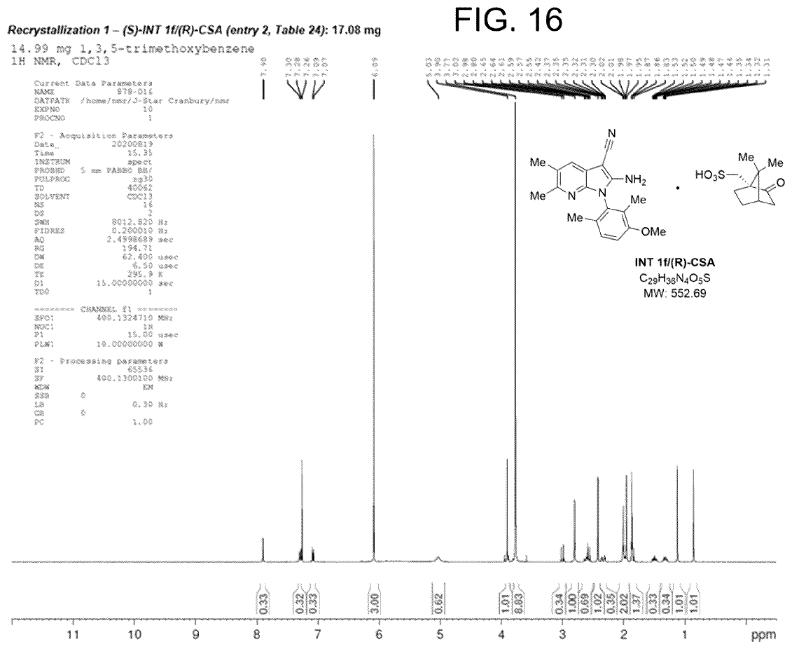

(S)-INT 1f/(R)-CSA (Reactive Crystallization-(S)-INT 1f/(R)-CSA, 3.73 g, 99.1 area %, 98.6 wt %=3.68 assay g, 85.3:13.7 e.r.) was dissolved in anisole (20 vol with respect to salt) at 80° C., cooled to 45-50° C., and slurry aged at 45-50° C. overnight at which point enantioenrichment had occurred (e.r. 95.5:4.5, Table 24). The suspension was cooled to ambient temperature and stirred over the weekend to afford (S)-INT 1f/(1R)-(−)-10-camphorsulfonic acid (2.84 g, 98.5 area %, 97.2 wt % by ¹H NMR vs. 1,3,5-trimethoxybenzene internal standard=2.76 assay g, 94.5:5.5 er) in 83% recovery. The ¹H NMR spectrum of (S)-INT 1f/(R)-CSA (entry 2, Table 24) is shown in FIG. 16.

TABLE 24
Recrystallization 1 - (S)-INT 1f/(R)-CSA (5 g)

| Entry | Description | e.r. |
|---|---|---|
| 1 | IPC | 95.5:4.5 |
| | 1. Age: 80° C. for 1 h | |
| | 2. Age: 45-50° C. overnight | |
| 2 | Isolated solid (2.84 g) | 94.5:5.5 |
| | 98.5 area % | |
| | 97.2 wt % | |

The impact of molar equivalents (mol equiv.) of acid used was investigated to see if this would impact the e.r. Reactive crystallization of racemic INT 1f and (R)-CSA in anisole with ratios 1:0.5, 1:1, 1:1.5 and 1:2 was performed. Interestingly, resolution was observed only in 1:1 ratio with the typical 83:17 e.r. observed for the isolated solid. To determine what ratio of INT 1f to (R)-CSA could be tolerated, the free base/acid ratios of 0.90, 0.95, 1.05, and 1.10 equiv. acid were investigated. All four ratios afforded ~87:13 e.r. after equilibrating at 45-50° C. overnight (Table 25), which is consistent with reactive crystallization using 1:1 ratio of free base/acid.

TABLE 25
(R)-CSA Equivalents Screen

| Entry | Acid (mol equiv.) | e.r. |
|---|---|---|
| 1 | 0.90 | 86.5:13.5 |
| 2 | 0.95 | 86.4:13.6 |
| 3 | 1.05 | 87.0:13.0 |
| 4 | 1.10 | 87.1:12.9 |

As recrystallization of INT 1f from CHCl₃ is not desirable for scale-up, reactive crystallization of unpurified racemic INT 1f was investigated. However, this crystallization failed to give enriched INT 1f. In fact, the material recovered was not only racemic but a 10:1 mix of CSA and INT 1f.

Likely, the charcoal treatment employed in the prior isolation step is critical for successful resolution if recrystallization of crude INT 1f is not performed. Hence, crude INT 1f from the same batch as above was charcoal (DARCO) treated (15.10 g, 88.0 wt %=13.29 g) to afford racemic INT 1f (8.81 g, 99.2 area %, 99.6 wt %=8.77 assay g) in 66% yield. This material successfully underwent reactive crystallization with (R)-CSA to afford the corresponding salt in 88.3:11.7 e.r. When charcoal treated INT 1f was doped to 11 wt % water (Table 26), reactive crystallization was also successful, affording the salt in 89.6:10.4 e.r. It is possible that the reactive crystallization failure of INT 1f may be due to the presence of an unspecified impurity (singlet at 2.01 ppm in ¹H NMR), which is removed by charcoal treatment or recrystallization (Table 27).

TABLE 26
Effect of Water on Reactive Crystallization

LCAP

| Entry | Description | Peak 1 | Peak 2 |
|---|---|---|---|
| 1 | 7.1 wt % H₂O | 88.3 | 11.7 |
| 2 | 11 wt % H₂O | 89.6 | 10.4 |

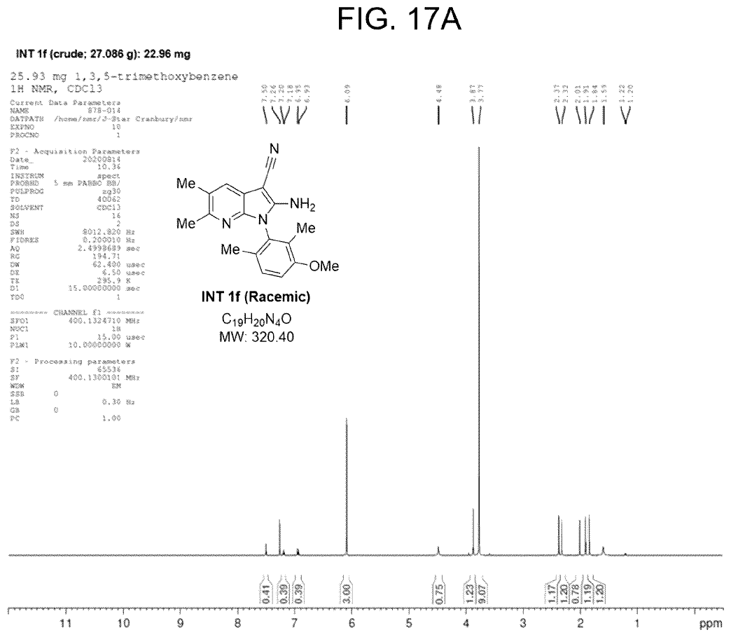

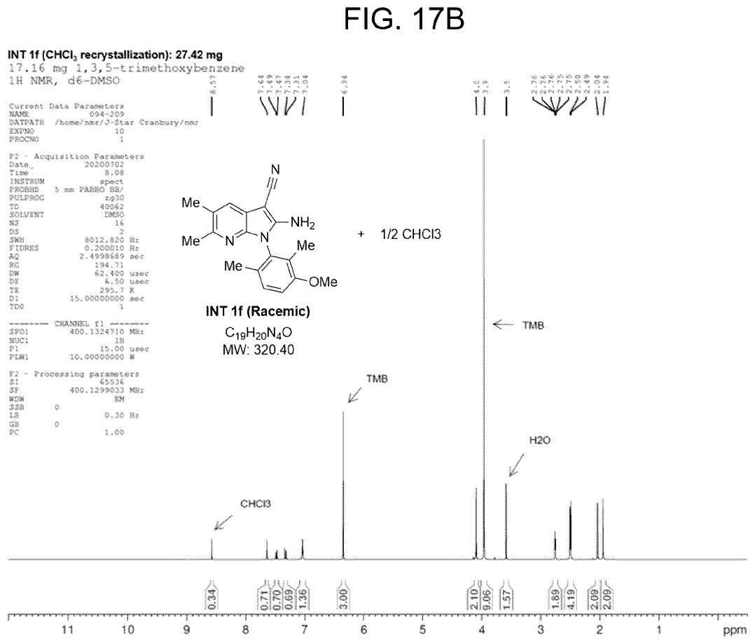

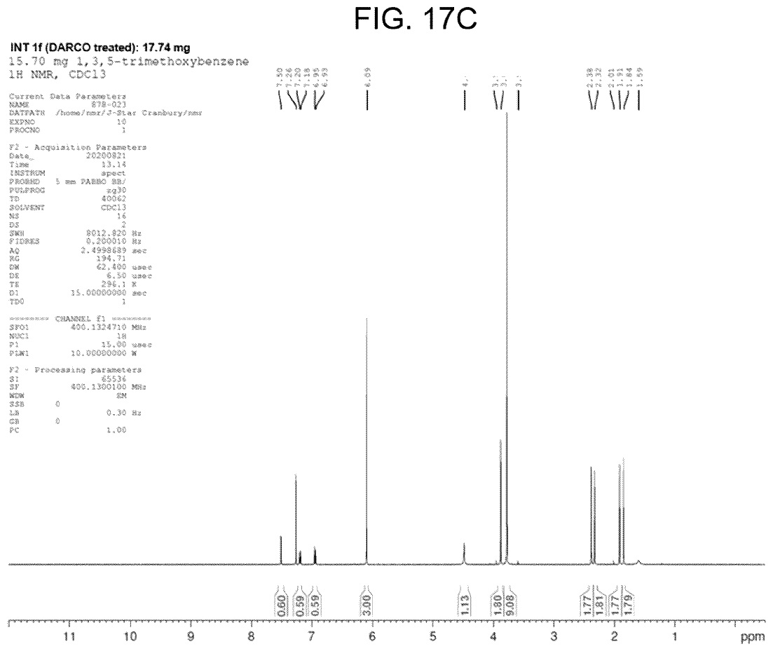

TABLE 27
INT 1f Lot Purity Comparison

| | | | | wt % | |
|---|---|---|---|---|---|
| | | H₂O wt % | LCAP | (¹H | ¹H |
| Entry | INT 1f | (KF) | (HPLC) | NMR) | NMR |
| 1 | Crude | 11.2 | 99.4 | 88.0 | FIG. 17A |
| 2 | CHCl₃ | 10.1 | 99.3 | 99.9 | FIG. 17B |
| | recrystallization | | | | |
| 3 | DARCO-treated | 7.1 | 99.2 | 99.6 | FIG. 17C |

From the seven acids screened it was found that the enriched salt formed from INT 1f and (R)-CSA when processed downstream gave the enantiomerically enriched(S)-Compound 1 without chiral erosion. The salt was prepared from a reactive crystallization in anisole between mole equivalent amounts of INT 1f and (R)-CSA to give enantiomerically enriched salt (85:15 e.r.) in 37% yield (74% recovery of available enantiomer). A recrystallization from anisole gave further enriched salt (95:5 e.r.) in 30% overall yield for the two-step process. The process could tolerate a plus or minus charge of 10% for the acid, as well as significant amount of water up to as much as 11%. Purification of racemic INT 1f either by recrystallization from a suitable solvent such as chloroform or purification through charcoal treatment for reactive crystallization in anisole to be successful.

### Example 14. INT 1f and(S)-INT 1f/(R)-CSA Physical Characteristics

Several important physical parameters of INT 1f and(S)-INT 1f/(R)-CSA were investigated.

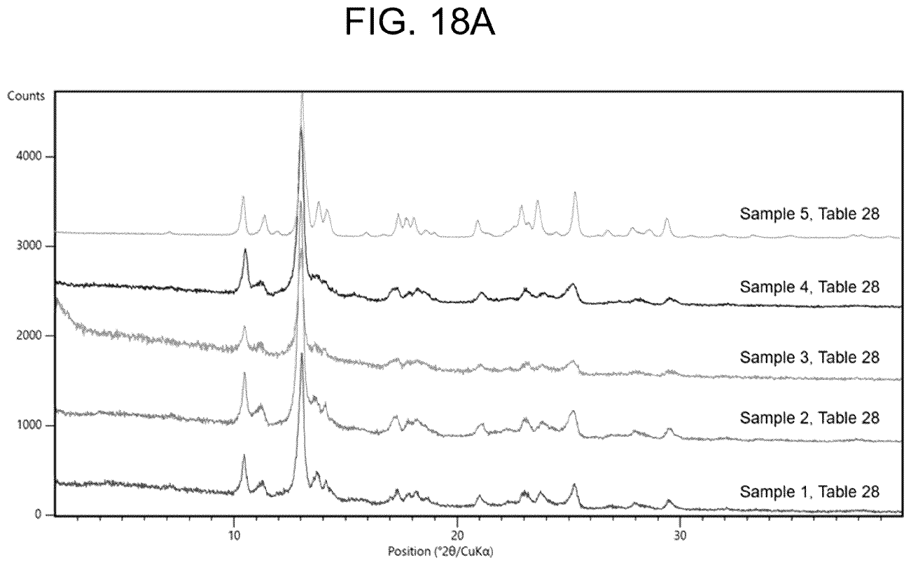

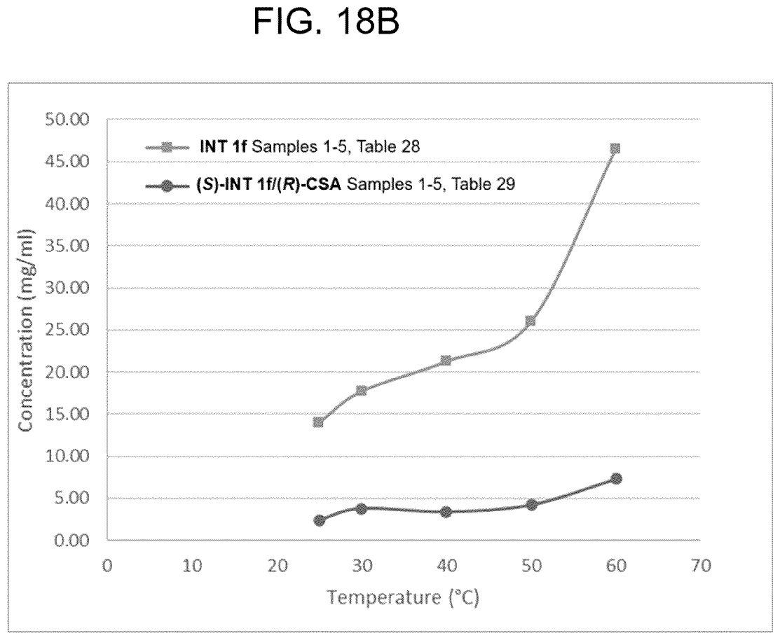

The solubility of racemic INT 1f (recrystallized from anisole) in anisole at various temperatures was investigated (Table 28). The XRPD spectra of each INT 1f sample in Table 28 is shown in FIG. 18A.

TABLE 28
Solubility of Racemic INT 1f (crystal form;
recrystallized from anisole) in anisole

| INT 1f | Temperature | Solubility in anisole |
|---|---|---|
| Sample No. | (° C.) | (mg/mL) |
| 1 | 60 | 46.5 |
| 2 | 50 | 25.99 |
| 3 | 40 | 21.26 |
| 4 | 30 | 17.72 |
| 5 | 25 | 14.01 |

The solubility of (S)-INT 1f/(R)-CSA salt in anisole at various temperatures was also investigated

### (Table 29).

TABLE 29
Solubility of (S)-INT 1f/(R)-CSA Salt in anisole

| (S)-INT 1f/(R)-CSA Salt | Temperature | Solubility in anisole |
|---|---|---|
| Sample No. | (° C.) | (mg/mL) |
| 1 | 60 | 7.29 |
| 2 | 50 | 4.20 |
| 3 | 40 | 3.37 |
| 4 | 30 | 3.78* |
| 5 | 21 | 2.39 |

*Outlier may be caused by experimental error

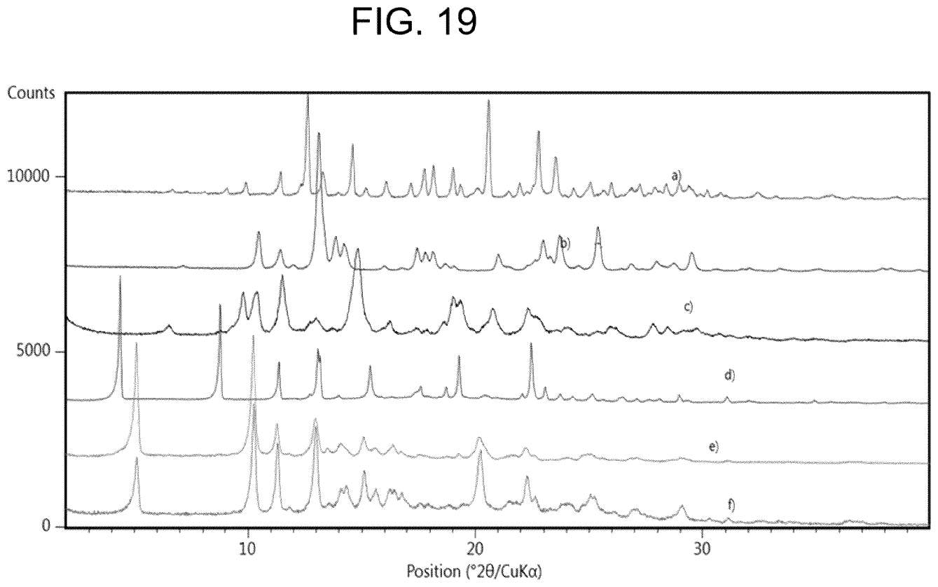

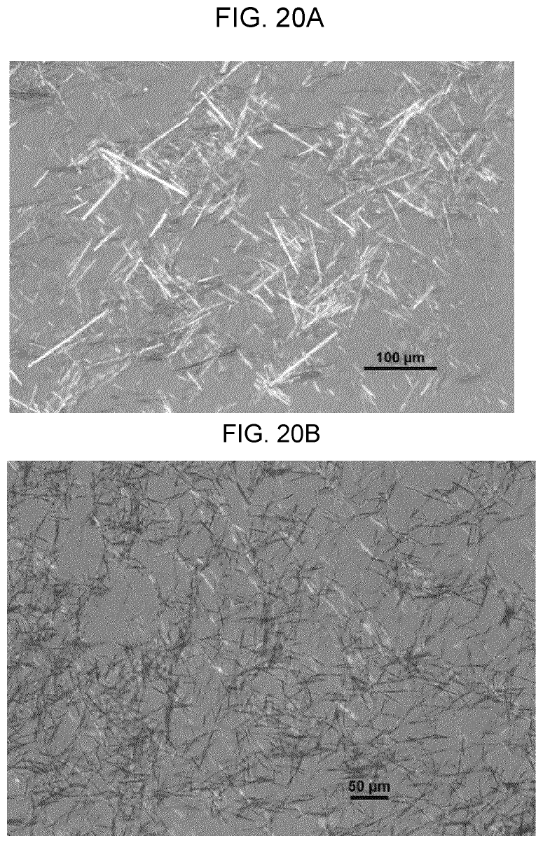

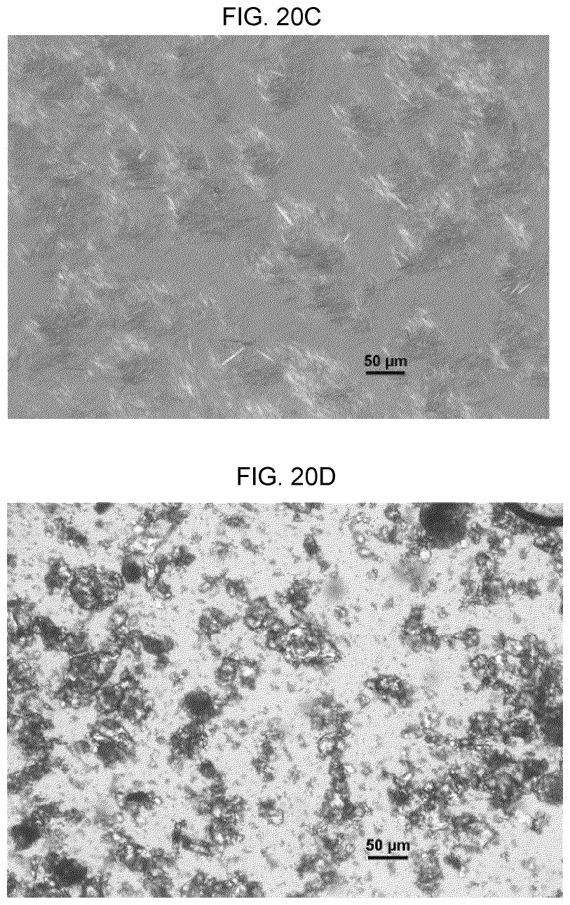

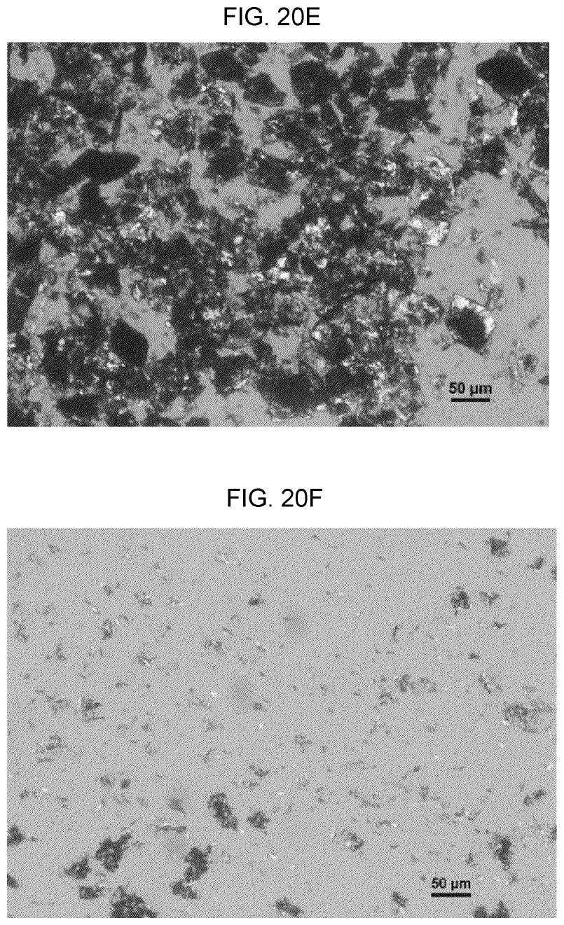

From the comparison in solubility (FIG. 20B), it seems unlikely that INT 1f would crystallize prior to the CSA salt form in the reactive crystallization process.

The crystal form and morphology of INT 1f and (R)-INT 1f/(S)-CSA from anisole and 1,3-dimethoxybenzene were investigated (FIG. 19, FIG. 20A, FIG. 20B, FIG. 20C, FIG. 20D, FIG. 20E, and FIG. 20F). It appears that from anisole a possible weak solvate is first isolated (form P1) that then collapses to form P2 upon drying in anisole. From 1,3-dimethoxybenzene, form P2 was obtained.

### Example 15. INT 1g and Compound 1 Chiral Acid Salts Summary

In this example, the resolution of INT 1g and Compound 1 via chiral salts is summarized. Solid state characterization is given in Table 30. Solubility of acid salts and ratio of enantiomers are given in Tables 31-38.

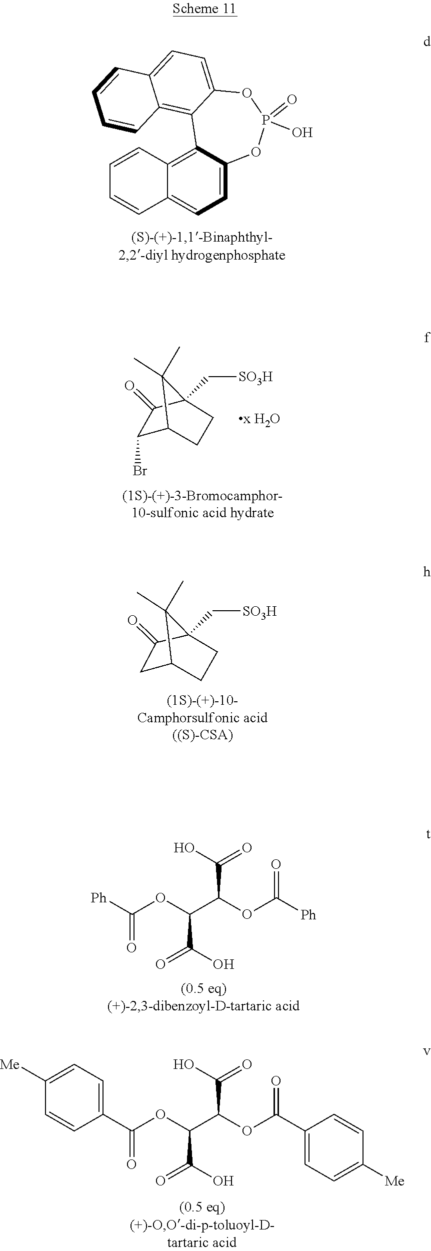

TABLE 30
Solid-state Characterization of INT 1g and Compound 1 salts

| Salt | XRPD | DSC |
|---|---|---|
| INT 1g/Acid d | Poorly crystalline | 1^{st} endotherm: 71.9° C. |
| | | (peak)/22.03 J/g |
| | | 2^{nd} endotherm: 126° C. |
| | | (peak)/2.51 J/g |
| INT 1g/Acid f | Amorphous | 1^{st} endotherm: 68.7° C. |
| | | (peak)/23.73 J/g |
| | | 2^{nd} endotherm: 123.6° C. |
| | | (peak)/5.39 J/g |
| INT 1g/Acid h | Amorphous | 1^{st} endotherm: 58.7° C. |
| | | (peak)/48.9 J/g |
| | | 2^{nd} endotherm: 113.5° C. |
| | | (peak)/13.88 J/g |
| Compound 1/Acid d | Poorly crystalline | 1^{st} endotherm: 72.3° C. |
| | | (peak)/64.33 J/g |
| | | 2^{nd} endotherm: 169.9° C. |
| | | (peak)/1.46 J/g |
| Compound 1/Acid f | Poorly crystalline | 1^{st} endotherm: 66.3° C. |
| | | (peak)/43.12 J/g |
| Compound 1/Acid h | Poorly crystalline | 1^{st} endotherm: 72.2° C. |
| | | (peak)/60.73 J/g |
| | | 2^{nd} endotherm: 133.7° C. |
| | | (peak)/1.46 J/g |

TABLE 31
INT 1g/(S)-(+)-1,1′-Binaphthyl-2,2′-diyl Hydrogen Phosphate Salt Screen (Acid d)

| | | | Isomer ratio of mother liquor | Approximate Solubility |
|---|---|---|---|---|
| Entry | Salt | Solvents | by HPLC | of starting material |
| 1 | INT 1g/Acid d | EtOH | Not tested. High solubility. | Very soluble |
| 2 | INT 1g/Acid d | EtOAc | Not tested. No solid precipitate. | Partially soluble |
| | | | (Gel obtained) | |
| 3 | INT 1g/Acid d | MeCN | Not tested. No solid precipitate. | Partially soluble |
| | | | (Gel obtained) | |
| 4 | INT 1g/Acid d | THF | 52:48 | * |
| 5 | INT 1g/Acid d | DCM | 50:50 | Very soluble |
| 6 | INT 1g/Acid d | 1,4-Dioxane | 49:51 | Soluble |
| 7 | INT 1g/Acid d | Toluene | 48:52 | * |
| 8 | INT 1g/Acid d | Acetone:water | Not tested. High solubility. | Very soluble |
| | | (9:1) | | |
| 9 | INT 1g/Acid d | EtOH:Heptane | Not tested. No solid precipitate. | Partially soluble |
| | | (1:1) | (Gel obtained) | |
| 10 | INT 1g/Acid d | Anisole | Not tested. High solubility. | Very soluble |

* Slurry, solubility not estimated. Experiment Procedures: Weight 50-60 mg of material into 4 mL vials. Add approximately 0.2 mL solvent and stir at RT for 3 days. Add more solvents to the vials with thick slurry and more solids to the vials with clear solution

TABLE 32
INT 1g/(1S)-(+)-3-Bromocamphor-10-sulfonic Acid Hydrate Salt Screen (Acid f)

| | | | Isomer ratio of mother liquor | Approximate Solubility |
|---|---|---|---|---|
| Entry | Salt | Solvents | by HPLC | of starting material |
| 1 | INT 1g/Acid f | EtOH | Not tested. High solubility. | Very soluble |
| 2 | INT 1g/Acid f | EtOAc | Not tested. High solubility. | Very soluble |
| 3 | INT 1g/Acid f | MeCN | Not tested. No solid precipitate. | Very Soluble |
| 4 | INT 1g/Acid f | THF | 50:50 | Very soluble |
| 5 | INT 1g/Acid f | DCM | Not tested. High solubility. | Very soluble |
| 6 | INT 1g/Acid f | 1,4-Dioxane | Not tested. High solubility. | Very Soluble |
| 7 | INT 1g/Acid f | Toluene | 44:56 | * |
| 8 | INT 1g/Acid f | Acetone:water | 20:20 (compound) | Very soluble |
| | | (9:1) | 30:30 (other)** | |
| 9 | INT 1g/Acid f | EtOH:Heptane | 55:45 | Soluble |
| | | (1:1) | | |
| 10 | INT 1g/Acid f | Anisole | not tested | Soluble |

* Slurry, solubility not estimated.
**Two other peaks were detected before INT 1g peaks.

TABLE 33
INT 1g/(S)-CSA Salt Screen (Acid h)

| | | | Isomer ratio of mother | Approximate Solubility |
|---|---|---|---|---|
| Entry | Salt | Solvents | liquor by HPLC | of starting material |
| 1 | INT 1g/Acid h | EtOH | Not tested. High solubility. | Very soluble |
| 2 | INT 1g/Acid h | EtOAc | 60:40 (ML) | Soluble |
| | | | 49:51 (solids) | |
| 3 | INT 1g/Acid h | MeCN | Not tested. High solubility. | Very soluble |
| 4 | INT 1g/Acid h | THF | Not tested. High solubility. | Very soluble |
| 5 | INT 1g/Acid h | DCM | Not tested. High solubility. | Very soluble |
| 6 | INT 1g/Acid h | 1,4-Dioxane | 50:50 (ML)** | Soluble |
| 7 | INT 1g/Acid h | Toluene | 58:42 (ML) | * |
| | | | 50:50(solids) | |
| 8 | INT 1g/Acid h | Acetone:water | 50:50 (ML) | Very soluble |
| | | (9:1) | 54:46 (solids) | |
| 9 | INT 1g/Acid h | EtOH:Heptane | 50:50 (ML) | Soluble |
| | | (9:1) | 50:50 (solids) | |
| 10 | INT 1g/Acid h | Anisole | 60:40 (ML) | Very soluble |
| | | | 50:50 (solids) | |

* Slurry, solubility not estimated.
**Not enough solids to filter therefore liquor assayed

TABLE 34
INT 1g/(+)-2,3-Dibenzoyl-D-tartaric Acid Salt Screen (Acid t)

| | | | Isomer ratio of mother | Approximate Solubility |
|---|---|---|---|---|
| Entry | Salt | Solvents | liquor by HPLC | of starting material |
| 1 | INT 1g/Acid t | EtOH | 50:50 | Soluble |
| 2 | INT 1g/Acid t | EtOAc | 50:50 | Soluble |
| 3 | INT 1g/Acid t | MeCN | 50:50 | Soluble |
| 4 | INT 1g/Acid t | THF | 50:50 | Soluble |
| 5 | INT 1g/Acid t | DCM | 50:50 | Soluble |
| 6 | INT 1g/Acid t | 1,4-Dioxane | 50:50 | Soluble |
| 7 | INT 1g/Acid t | Toluene | 50:50 | Soluble |
| 8 | INT 1g/Acid t | Acetone:water | 50:50 | Very soluble |
| | | (9:1) | | |
| 9 | INT 1g/Acid t | EtOH:Heptane | 50:50 | Soluble |
| | | (9:1) | | |
| 10 | INT 1g/Acid t | Anisole | 50:50 | Soluble |

TABLE 35
INT 1g/(+)-O,O′-Di-p-toluoyl-D-tartaric Acid Salt Screen (Acid v)

| | | | Isomer ratio of mother | Approximate Solubility |
|---|---|---|---|---|
| Entry | Salt | Solvents | liquor by HPLC | of starting material |
| 1 | INT 1g/Acid v | EtOH | 50:50 | Soluble |
| 2 | INT 1g/Acid v | EtOAc | — | Soluble |
| 3 | INT 1g/Acid v | MeCN | 50:50 | Soluble |
| 4 | INT 1g/Acid v | THF | 50:50 | Soluble |
| 5 | INT 1g/Acid v | DCM | — | Highly soluble |
| 6 | INT 1g/Acid v | 1,4-Dioxane | 50:50 | Soluble |
| 7 | INT 1g/Acid v | Toluene | 50:50 | Soluble |
| 8 | INT 1g/Acid v | Acetone:water | 50:50 | Soluble |
| | | (9:1) | | |
| 9 | INT 1g/Acid v | EtOH:Heptane | 50:50 | Soluble |
| | | (9:1) | | |
| 10 | INT 1g/Acid v | Anisole | 50:50 | Highly soluble |

— No solids afforded for LC analysis. More material was added to the samples

TABLE 36
Compound 1/(S)-(+)-1,1′-Binaphthyl-2,2′-diyl Hydrogen Phosphate Salt Screen (Acid d)

| | | | | Approximate |
|---|---|---|---|---|
| | | | Isomer ratio of mother | Solubility of starting |
| Entry | Salt | Solvents | liquor by HPLC | material |
| 1 | Compound 1/Acid d | EtOH | 49:51 (ML) | * |
| 2 | Compound 1/Acid d | EtOAc | 48:52 (ML) | * |
| 3 | Compound 1/Acid d | MeCN | 49:51 (ML) | * |
| 4 | Compound 1/Acid d | THF | 49:51 (ML) | * |
| 5 | Compound 1/Acid d | DCM | Not tested. No solid | Partially soluble |
| | | | precipitate. (Gel obtained) | |
| 6 | Compound 1/Acid d | 1,4-Dioxane | 26:74 (ML) | * |
| | | | 50:50 (solids) | |
| 7 | Compound 1/Acid d | Toluene | 39:61 (ML) | * |
| | | | 50:50 (solids) | |
| 8 | Compound 1/Acid d | Acetone:water | 50:50 (ML) | * |
| | | (9:1) | | |
| 9 | Compound 1/Acid d | EtOH:Heptane | Not tested. No solid | Partially soluble |
| | | (1:1) | precipitate. (Gel obtained) | |
| 10 | Compound 1/Acid d | Anisole | Not tested. No solid | Partially soluble |
| | | | precipitate. (Gel obtained) | |

* Slurry, solubility not estimated.

TABLE 37
Compound 1/(1S)-(+)-3-Bromocamphor-10-sulfonic Acid Hydrate Salt Screen (Acid f)

| | | | Isomer ratio of mother | Approximate Solubility |
|---|---|---|---|---|
| Entry | Salt | Solvents | liquor by HPLC | of starting material |
| 1 | Compound 1/Acid f | EtOH | 50:50 | Partially soluble |
| 2 | Compound 1/Acid f | EtOAc | 42:58 | * |
| 3 | Compound 1/Acid f | MeCN | 51:49 | Partially soluble |
| 4 | Compound 1/Acid f | THF | 50:50 | Soluble |
| 5 | Compound 1/Acid f | DCM | 49:51 | Partially soluble |
| 6 | Compound 1/Acid f | 1,4-Dioxane | 50:50 | * |
| 7 | Compound 1/Acid f | Toluene | 58:42 | * |
| 8 | Compound 1/Acid f | Acetone:water | 52:48 | Soluble |
| | | (9:1) | | |
| 9 | Compound 1/Acid f | EtOH:Heptane | 50:50 | Soluble |
| | | (1:1) | | |
| 10 | Compound 1/Acid f | Anisole | 45:55 | Soluble |

* Slurry, solubility not estimated.

TABLE 38
Compound 1/(S)-CSA Salt Screen (Acid h)

| | | | Isomer ratio of mother | Approximate Solubility |
|---|---|---|---|---|
| Entry | Salt | Solvents | liquor by HPLC | of starting material |
| 1 | Compound 1/Acid h | EtOH | 50:50 (ML) | Soluble |
| 2 | Compound 1/Acid h | EtOAc | 35:65 (ML) | * |
| 3 | Compound 1/Acid h | MeCN | 50:50 (ML) | * |
| 4 | Compound 1/Acid h | THF | 47:53 (ML) | * |
| 5 | Compound 1/Acid h | DCM | 44:56 (ML) | Partially soluble |
| 6 | Compound 1/Acid h | 1,4-Dioxane | 52:48 (ML) | * |
| 7 | Compound 1/Acid h | Toluene | 50:50 (solids) | * |
| 8 | Compound 1/Acid h | Acetone:water | 52:48 (ML) | Soluble |
| | | (9:1) | | |
| 9 | Compound 1/Acid h | EtOH:Heptane | 48:52 (ML) | * |
| | | (9:1) | | |
| 10 | Compound 1/Acid h | Anisole | 49:51 (ML) | * |

* Slurry, solubility not estimated.

In contrast to the results observed for INT 1f in Example 10, appreciable enantioenrichment was not observed for any salt of INT 1g and a chiral acid, both in recovered solids and in the mother liquor. The highest enantiopurity found for INT 1g chiral acid salts was a 60:40 isomer ratio observed in the mother liquor of the combination of INT 1g with acid h using either anisole or EtOAc as a solvent (Table 33, entries 2 and 10). Additionally, appreciable enantioenrichment was not observed for any salt of Compound 1 and a chiral acid, both in recovered solids and in the mother liquor. The highest enantiopurity found for Compound 1 chiral acid salts was a 26:74 isomer ratio observed in the mother liquor of the combination of Compound 1 with acid d using 1,4-dioxane as a solvent (Table 36, entry 6).

### Example 16. A procedure for the synthesis of (S)-2-amino-1-(3-hydroxy-2,6-dimethylphenyl)-5,6-dimethyl-1H-pyrrolo-[2,3-b]pyridine-3-carboxamide ((S)-Compound 1)

This example describes a batch procedure for the synthesis of (S)-2-amino-1-(3-hydroxy-2,6-dimethylphenyl)-5,6-dimethyl-1H-pyrrolo-[2,3-b]pyridine-3-carboxamide ((S)-Compound 1).

The synthetic route starts from 2,6-dimethyl-4-methoxyaniline (INT 2a) and 2,3-dibromo-5,6-dimethylpyridine (INT 2b) and comprises four chemical transformations (see Scheme 12).

The first step involves Buchwald-Hartwig coupling of INT 2a and INT 2b to 3-bromo-N-(3-methoxy-2,6-dimethylphenyl)-5,6-dimethylpyridin-2-amine (INT 1e). This intermediate is reacted with malononitrile to racemic 2-amino-1-(3-methoxy-2,6-dimethyl-phenyl)-5,6-dimethyl-1H-pyrrolo[2,3-b]pyridine-3-carbonitrile (INT 1f). (S)-INT 1f/(R)-CSA salt can be crystallized in situ from INT 1f and (R)-CSA with around 85% chiral purity. Additional rounds of slurrying will improve this to over 95%. Intermediate salt(S)-INT 1f/(R)-CSA is converted to crude(S)-Compound 1, which is recrystallized to afford final product.

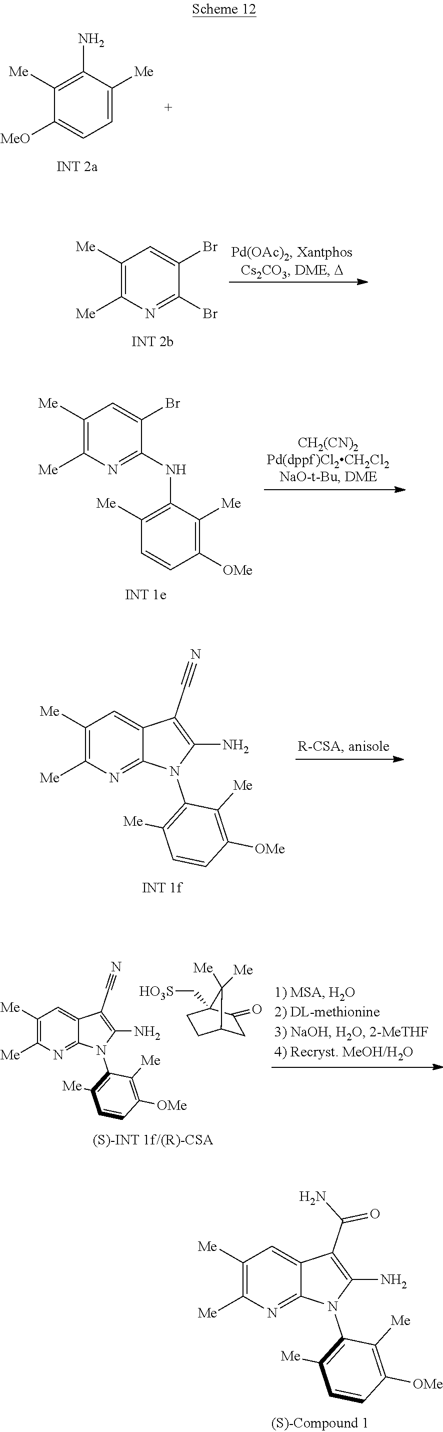

Raw materials: All key raw materials, reagents, solvents, and process chemicals were used as received from commercial suppliers.

### Step 1: Synthesis of 3-Bromo-N-(3-methoxy-2,6-dimethylphenyl)-5,6-dimethylpyridin-2-amine (INT 1e)

A mixture of 3-methoxy-2,6-dimethylaniline (INT 2a, 95.0 g, 0.628 mol), 2,3-dibromo-5,6-dimethylpyridine (INT 2b, 182 g, 0.685 mol), Cs₂CO₃ (409 g, 1.26 mol), Xantphos (21.8 g, 37.7 mmol), Pd(OAc)₂ (2.82 g, 12.6 mmol), and DME (1.9 L) is stirred at reflux for 16 h. After completion of reaction the mixture is cooled to ambient temperature and filtered through a pad of diatomite (47.5 g). The filter cake is washed with EtOAc (3×250 mL). The combined filtrate and washing liquids are stirred with activated charcoal (95.0 g) at ambient temperature for 1.5 h. The decolorized solution is filtered, and the filter cake is washed with EtOAc (240 mL). The combined filtrate and washing liquids are concentrated under vacuum to approx. 700 mL, after which a solvent switch to EtOH is performed by repeated addition of EtOH (715 mL) and concentration to 700 mL. Then, more EtOH (425 mL) is added, and the resulting mixture is heated at reflux for about 30 min, cooled down to ambient temperature, stirred overnight, and filtered. The filter cake is washed with EtOH (240 mL) and dried under vacuum at 50° C. to afford INT 1e as a yellowish green solid (163 g) in 77% yield. ¹H NMR (400 MHz, DMSO) δ 7.57 (s, 1H), 7.26 (s, 1H), 7.03 (d, J=8.0 Hz, 1H), 6.77 (d, J=8.3 Hz, 1H), 3.76 (s, 3H), 2.06 (s, 3H), 2.03 (s, 6H), 1.94 (s, 3H). ¹³C NMR (101 MHz, DMSO) δ 156.14, 153.90, 151.45, 141.56, 139.05, 128.28, 127.24, 124.64, 121.57, 108.46, 101.20, 55.87, 22.25, 18.37, 17.59, 11.83.

### Step 2: Synthesis of 2-Amino-1-(3-methoxy-2,6-dimethylphenyl)-5,6-dimethyl-1H-pyrrolo[2,3-b]-pyridine-3-carbonitrile (INT 1f)

To a solution of malononitrile (644 g, 9.74 mol) in DME (32 L) is added portion wise NaO-t-Bu (895 g, 9.31 mol) at 0° C. This is followed by addition of INT 1e (1.57 kg, 4.68 mol) and Pd(dppf)Cl₂·CH₂Cl₂ (204.1 g, 0.250 mol) at ambient temperature. The resulting mixture is heated at reflux for 18 h. After completion of reaction the reaction mass is cooled to 35° C. and filtered through a pad of diatomite (1.57 kg). The filter cake is washed with DME (3×3.0 L). The combined filtrate and washing liquids are stirred with activated charcoal (1.75 kg) at 60° C. for 2 h. The decolorized solution is filtered, and the filter cake is washed with EtOAc (2×5.2 L). The decolorization procedure is repeated once. The combined filtrate and washing liquids are concentrated under vacuum to 11 L, after which a solvent switch to i-PrOH is performed by repeated addition of i-PrOH (10 L) and concentration to 11 L. The resulting mixture is heated at reflux for about 30 min, cooled down to ambient temperature, stirred overnight, and filtered. The filter cake is washed with i-PrOH (4.0 L) and dried under vacuum at 50° C. to afford INT 1f as a light-yellow solid (1.13 kg) in 76% yield. ¹H NMR (400 MHz, DMSO) δ 7.39 (s, 1H), 7.22 (d, J=8.5 Hz, 1H), 7.07 (d, J=8.5 Hz, 1H), 6.78 (s, 2H), 3.84 (s, 3H), 2.25 (d, J=5.1 Hz, 6H), 1.79 (s, 3H), 1.70 (s, 3H). ¹³C NMR (101 MHz, DMSO) δ 156.53, 153.19, 147.39, 143.51, 132.59, 129.06, 128.60, 126.18, 125.25, 124.53, 119.06, 117.58, 112.00, 59.05, 56.09, 22.31, 19.20, 17.32, 11.29.

### Step 3: Synthesis of (S)-2-Ammonium-1-(3-methoxy-2,6-dimethylphenyl)-5,6-dimethyl-1H-pyrrolo[2,3-b]-pyridine-3-carbonitrile/(R)-CSA ((S)-INT 1f/(R)-CSA)

A mixture of INT 1f (1.12 kg, 3.50 mol), (R)-CSA (818 g, 3.52 mol), and anisole (32 L) is heated at 85° C. for 1 h and cooled to 50° C., after which crystal seeds (2.2 g, 7.0 mmol) are added. The mixture is stirred at 50° C. for 14 h, cooled to 10° C. over a period of 5 h, aged at this temperature for 16 h, and filtered. The filter cake is washed with anisole (2.25 L) and dried on the centrifuge for 1 h to afford crude, wet (S)-INT 1f/(R)-CSA (1.46 kg). A suspension of crude(S)-INT 1f/(R)-CSA in anisole (16 L) is heated at 85° C. for 2 h, cooled to 10° C. over a period of several h, aged at this temperature for 16 h, and filtered. The filter cake is washed with anisole (1.8 L) and dried under vacuum at 50° C. to afford (S)-INT 1f/(R)-CSA with a chiral purity of 97.5% as an off-white solid (758 g) in 39% yield. ¹H NMR (400 MHz, DMSO) δ 7.55 (s, 1H), 7.25 (d, J=8.5 Hz, 1H), 7.11 (d, J=8.5 Hz, 1H), 3.85 (s, 3H), 2.95 (d, J=14.7 Hz, 1H), 2.68-2.55 (m, 1H), 2.48-2.51 (m, 1H), 2.28 (d, J=4.7 Hz, 6H), 2.25-2.18 (m, 1H), 1.96 (t, J=4.5 Hz, 1H), 1.91-1.82 (m, 2H), 1.79 (s, 3H), 1.71 (s, 3H), 1.39-1.23 (m, 2H), 1.04 (s, 3H), 0.75 (s, 3H). ¹³C NMR (101 MHz, DMSO) δ 216.42, 156.61, 153.38, 146.07, 142.02, 131.90, 129.03, 128.82, 126.27, 125.98, 125.78, 119.94, 117.10, 112.37, 59.38, 58.57, 56.14, 47.60, 47.36, 42.68, 42.60, 26.84, 24.64, 21.28, 20.48, 19.97, 18.98, 17.24, 11.27.

### Step 4: Synthesis of (S)-2-amino-1-(3-hydroxy-2,6-dimethylphenyl)-5,6-dimethyl-1H-pyrrolo[2,3-b]pyridine-3-carboxamide ((S)-Compound 1)

To a mixture of methanesulfonic acid (3.5 L) and water (65.6 mL, total water content 1.60% w/w) is added(S)-INT 1f/(R)-CSA (750 g, 1.36 mol) in portions at 45° C. Stirring is continued at this temperature for 4 h. After completion of reaction, the resulting mass is cooled to ambient temperature and DL-methionine (810 g, 5.43 mol) is added. The reaction mixture is stirred at 75° C. for 18 h, cooled to ambient temperature, and quenched by drop wise addition to chilled water (15 L) keeping the temperature below 20° C. The quenched mixture is diluted with water (5.6 L) and stirred with activated charcoal (75.0 g) at ambient temperature for 3 h. The decolorized solution is filtered through a pad of diatomite (375 g), and the filter cake is washed with water (7.5 L). Then, 2-MeTHF (3.3 L) is added to the combined filtrate and washing liquid. The pH of the biphasic system is adjusted to 8-9 with aqueous NaOH (30% w/w, approx. 6 L). The suspension that is obtained is heated at 55° C. for 30 min, cooled to ambient temperature, stirred for 10 h, and filtered. The filter cake is washed with water (3×2.3 L) dried under vacuum at 50° C. to afford crude (S)-Compound 1 as a light-brown solid (410 g, chemical purity 98.8% area, chiral purity 97.4%) in 93% yield.

Crude (S)-Compound 1 (270 g, 0.832 mol) is dissolved in methanol (3.4 L) at 65° C., the resulting mixture is cooled to 55° C. and filtered (warm) to remove insoluble material. The filter cake is washed with methanol (680 mL). Water (270 mL) is added to the filtrate at 55° C., which is then cooled to 0° C. Seed crystals (16 mg as a suspension in methanol/water 1:1 v/v (270 mL)) are added, and the subsequent mixture is stirred at 0° C. for 3 h. Water (3.8 L) is dosed over a period of several hours, after which stirring is continued at 0° C. for 10 h. The resulting suspension is filtered. The filter cake is washed with methanol/water (1:1 v/v, 405 mL) and dried under vacuum at 50° C. to afford (S)-Compound 1 (217 g) as a light-brown solid (217 g, chemical purity 99.6% area, chiral purity 99.9%) in 80% yield from crude(S)-Compound 1. ¹H NMR (400 MHz, DMSO) δ 9.48 (s, 1H), 7.83 (s, 1H), 7.05 (d, J=8.1 Hz, 1H), 6.91 (d, J=8.1 Hz, 1H), 6.71 (s, 2H), 6.65 (s, 2H), 2.25 (d, J=8.1 Hz, 6H), 1.75 (s, 3H), 1.67 (s, 3H). ¹³C NMR (101 MHz, DMSO) δ 168.91, 154.62, 152.27, 145.59, 143.95, 133.10, 128.26, 127.52, 125.67, 124.47, 124.11, 116.59, 115.97, 83.56, 22.22, 19.34, 17.38, 11.35.

### Example 17. Alternative procedure for the synthesis of (S)-2-amino-1-(3-hydroxy-2,6-dimethylphenyl)-5,6-dimethyl-1H-pyrrolo-[2,3-b]pyridine-3-carboxamide ((S)-Compound 1)

This example describes a batch procedure for the synthesis of (S)-2-amino-1-(3-hydroxy-2,6-dimethylphenyl)-5,6-dimethyl-1H-pyrrolo-[2,3-b]pyridine-3-carboxamide ((S)-Compound 1).

The synthetic route starts from 2,6-dimethyl-4-methoxyaniline (INT 2a) and 2,3-dibromo-5,6-dimethylpyridine (INT 2b) and comprises four chemical transformations (see Scheme 13).

The first step involves Buchwald-Hartwig coupling of INT 2a and INT 2b to 3-bromo-N-(3-methoxy-2,6-dimethylphenyl)-5,6-dimethylpyridin-2-amine (INT 1e). This intermediate is reacted with malononitrile to racemic 2-amino-1-(3-methoxy-2,6-dimethyl-phenyl)-5,6-dimethyl-1H-pyrrolo[2,3-b]pyridine-3-carbonitrile (INT 1f). (S)-INT 1f/(R)-CSA salt can be crystallized in situ from INT 1f and (R)-CSA followed by additional rounds of slurrying with over 97% chiral purity. Intermediate salt(S)-INT 1f/(R)-CSA is converted to crude (S)-Compound 1, which is recrystallized to afford final product.

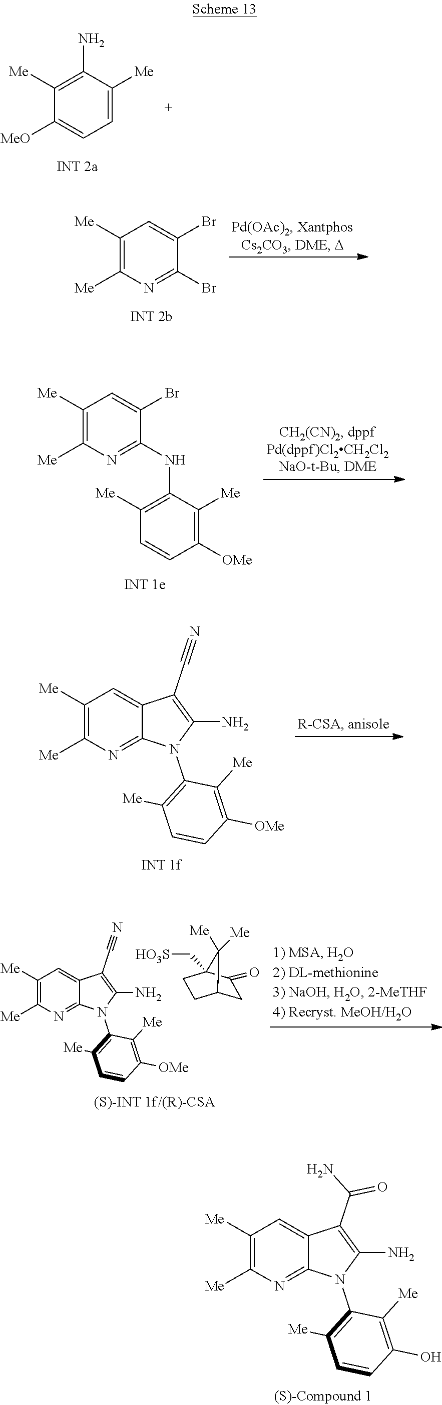

Raw materials: All key raw materials, reagents, solvents, and process chemicals were used as received from commercial suppliers.

### Step 1: Synthesis of 3-Bromo-N-(3-methoxy-2,6-dimethylphenyl)-5,6-dimethylpyridin-2-amine (INT 1e)

A mixture of 3-methoxy-2,6-dimethylaniline (INT 2a, 80.0 g, 0.529 mol) in 1,2-dimethoxyethane (DME; 1.38 L) is stirred for 5-10 min at 20-30° C., after which 2,3-dibromo-5,6-dimethylpyridine (INT 2b, 152.8 g, 0.576 mol), Cs₂CO₃ (258.6 g, 0.794 mol), Xantphos (18.4 g, 31.7 mmol), and Pd(OAc)₂ (2.4 g, 11 mmol) are added under nitrogen. The resulting mixture is stirred at 80-85° C. for 20-24 h, cooled to 20-30° C., and filtered through a pad of diatomite (20.0 g, 0.25 rel. weights). The filter cake is washed with DME (2×460 mL, then 276 mL). The combined filtrate and washing liquids are concentrated under vacuum at 40-50° C. to 600 mL (7-8 rel. volumes), after which water (240 mL) is added at 60-75° C. The resulting mixture is cooled to 20-30° C., aged for 10-15 h, and filtered. The filter cake is washed with a mixture of DME (276 mL) and water (120 mL) and dried under nitrogen protection for 30-60 minutes at 20-30° C. to afford crude INT 1e as a yellow solid (164.2 g, 93% yield).

A mixture of crude INT 1e (164.2 g) in ethanol (800 mL) is heated at 75-78° C. for 1-2 hours, then cooled to 20-30° C., aged for 10-15 h, and filtered. The filter cake is washed with ethanol (200 mL) and dried under vacuum at 45-55° C. to afford INT 1e as a yellow solid (145.2 g, 82% yield).

### Step 2: Synthesis of 2-Amino-1-(3-methoxy-2,6-dimethylphenyl)-5,6-dimethyl-1H-pyrrolo[2,3-b]-pyridine-3-carbonitrile (INT 1f)

A solution of malononitrile (53.2 g, 0.805 mol) in DME (4.5 L) under nitrogen is stirred for 5-10 min at 20-30° C. and then cooled to 0-10° C. (target 5° C.). Sodium tert-butoxide (96.8 g, 1.01 mmol) is added in 5 portions at 0-10° C. The reactor mixture is warmed up to 20-30° C. and aged at that temperature for 30-40 min. Then, INT 1e (225.0 g, 0.671 mol), 1,1′-bis(diphenylphosphino) ferrocene (dppf; 7.44 g, 0.0134 mol) and Pd(dppf)Cl₂·CH₂Cl₂ (10.96 g, 13.4 mmol) are added. The resulting mixture is heated at 83-87° C. for 18-24 hours, cooled to 30-50° C. and filtered through a pad of diatomite (112.5 g). The filter cake is washed with DME (2×1.3 L). The combined filtrate and washing liquid are concentrated under vacuum at 40-50° C. to approx. 1,700 mL (7-8 rel. volumes), after which water (675 mL) is added. The resulting suspension is heated at 60-75° C. for 1-2 hours, then cooled to 20-30° C., aged for 10-15 h, and filtered. The filter cake is washed with a mixture of DME (620 mL) and water (270 mL) and dried under nitrogen protection for 30-60 minutes at 20-30° C. to afford crude INT 1f as an off-white solid (189.5 g), which is suspended in 2-propanol (2.02 L) and heated at 80-83° C. for 30-45 min. The mixture is cooled to 18-28° C., aged for 2-4 h, and filtered. The filter cake is washed with 2-propanol (572 mL) and dried under vacuum at 45-55° C. to afford INT 1f as a white solid (153.0 g, 74% yield).

### Step 3: Synthesis of (S)-2-Ammonium-1-(3-methoxy-2,6-dimethylphenyl)-5,6-dimethyl-1H-pyrrolo[2,3-b]-pyridine-3-carbonitrile/(R)-CSA ((S)-INT 1f/(R)-CSA)

A mixture of INT 1f (148 g, 0.462 mol), (R)-CSA (107.3 g, 0.462 mol), and anisole (2.97 L) is heated at 85° C. for 1 h and cooled to 45-50° C., after which crystal seeds (296 mg, 0.536 mmol) are added. The mixture is stirred at 48-52° C. for 4-6 h, is cooled to 7-13° C. at a cooling rate of 5-10° C./h and aged at this temperature for approx. 16 h. The suspension is filtered and the filter cake is washed with anisole (297 mL) and dried on the filter or centrifuged for 1 h to afford wet crude(S)-INT 1f/(R)-CSA. A suspension of crude (S)-INT 1f/(R)-CSA in anisole (2.05 L) is stirred at 20-30° C. for 5-10 min, heated at 80-85° C. for 1-2 h, cooled to 7-13° C., and subsequently aged at this temperature for 16 h. The suspension is filtered, and the filter cake is washed with anisole (296 g, 2.0 rel. weights) and dried under vacuum at 45-55° C. to afford (S)-INT 1f/(R)-CSA as an off-white solid (106.2 g, 42% yield) with a chiral purity of 97.4%.

### Step 4: Synthesis of (S)-2-amino-1-(3-hydroxy-2,6-dimethylphenyl)-5,6-dimethyl-1H-pyrrolo[2,3-b]pyridine-3-carboxamide ((S)-Compound 1)

To a mixture of methanesulfonic acid (657.4 g) and water (6.11 mL, total water content 1.60% w/w) is added(S)-INT 1f/(R)-CSA (95.0 g, 0.172 mol) in 16 portions at 40-48° C. Stirring is continued at this temperature for 8 h, after which the reaction mixture is cooled down to 15-25° C. and DL-Methionine (102.6 g, 0.688 mol) is added. The resulting mixture is heated at 70-78° C. for 18-24 h, cooled to 15-25° C. and then slowly added to pre-cooled (0-10° C.) water (2.38 kg). The quenched mixture is diluted with water (475 mL) and 2-MeTHF (334 mL). The pH is adjusted to 7-9 by addition of aqueous NaOH (approx. 30% w/w). The suspension is aged at 30-35° C. for 20-60 min and at 15-25° C. for 10-15 h, and filtered. The filter cake is washed with water (3×475 mL) and dried under vacuum at 45-55° C. to give a brown solid (54.2 g, 91% yield). A suspension of the above solid (53.0 g) in THF (425 mL) is heated at 65-70° C. for 20-40 min, then cooled to −3-3° C., aged for 20-24 h, and filtered. The filter cake is washed with THF (2×53 mL) and dried under vacuum at 45-55° C. to afford crude(S)-Compound 1 as a light brown solid (57.5 g, 108% yield).

To crude(S)-Compound 1 (58.3 g, 0.180 mol) in methanol (736 mL) is added activated carbon (5.83 g). The suspension is stirred at 20-30° C. for 5-10 min and at 60-66° C. for 1-2 h, cooled down to 50-55° C. and filtered to remove insoluble matter. The filter cake is washed with methanol (147 mL). Water (29.2 mL) is added slowly to the combined filtrate and washing liquid at 50-55° C. The resulting mixture is cooled to 3-7° C., after which a suspension of crystal seeds (0.58 g, 1.8 mmol) in MeOH/water (4:5 v/v, 58 mL) is charged. Stirring is continued at 3-7° C. for 3 h, which is followed by the addition of water (857 mL) at 3-7° C. over a period of 4-9 h. The resulting suspension is aged at this temperature for 8-12 h and filtered. The filter cake is washed with a mixture of MeOH (49 mL) and water (43.7 mL) twice and dried under vacuum at 45-55° C. to afford (S)-Compound 1 as a yellowish to off-white solid (44.3 g, 76% yield) with a purity of >99.9% and chiral purity of 99.7%.

### Example 18. A procedure for the synthesis of (S)-2-amino-1-(3-hydroxy-2,6-dimethylphenyl)-5,6-dimethyl-1H-pyrrolo-[2,3-b]pyridine-3-carboxamide ((S)-Compound 1)

This example describes a procedure for GMP manufacture of (S)-2-amino-1-(3-hydroxy-2,6-dimethylphenyl)-5,6-dimethyl-1H-pyrrolo-[2,3-b]pyridine-3-carboxamide ((S)-Compound 1).

The GMP manufacture of (S)-2-amino-1-(3-hydroxy-2,6-dimethylphenyl)-5,6-dimethyl-1H-pyrrolo[2,3-b]pyridine-3-carboxamide (1) starts from 2,6-dimethyl-4-methoxyaniline (2) and 2,3-dibromo-5,6-dimethylpyridine (3) and comprises four chemical transformations (see Scheme 14).

The first step involves Buchwald-Hartwig coupling of 2 and 3 to 3-bromo-N-(3-methoxy-2,6-dimethylphenyl)-5,6-dimethylpyridin-2-amine (4). This intermediate is reacted with malononitrile to racemic 2-amino-1-(3-methoxy-2,6-dimethyl-phenyl)-5,6-dimethyl-1H-pyrrolo[2,3-b]pyridine-3-carbonitrile (INT 1f). (S)-2-ammonium-1-(3-methoxy-2,6-dimethyl-phenyl)-5,6-dimethyl-1H-pyrrolo[2,3-b]pyridine-3-carbonitrile/(R)-CSA ((S)-INT 1f/(R)-CSA) can be crystallized in situ from INT 1f and (R)-CSA with around 85% chiral purity. Additional rounds of slurrying will improve this to over 95%. Intermediate salt(S)-INT 1f/(R)-CSA is converted to crude(S)-Compound 1, which is recrystallized to afford final product.

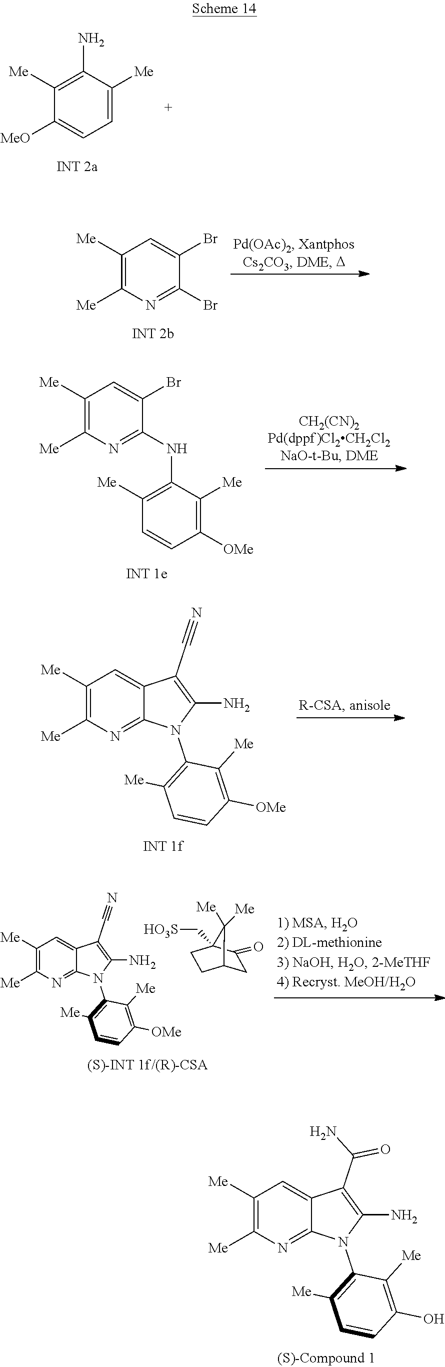

Raw materials: All key raw materials, reagents, solvents, and process chemicals were used as received from commercial suppliers.

### Step 1: Synthesis of 3-Bromo-N-(3-methoxy-2,6-dimethylphenyl)-5,6-dimethylpyridin-2-amine (INT 1e)

A mixture of 3-methoxy-2,6-dimethylaniline (INT 2a, 8.08 kg, 53.4 mol), 2,3-dibromo-5,6-dimethylpyridine (INT 2b, 15.5 kg, 58.5 mol), Cs₂CO₃ (35.0 kg, 107 mol), Xantphos (1.86 kg, 3.21 mol), Pd(OAc)₂ (243 g, 1.08 mol), and DME (160 L) is stirred at reflux for 16 h. After completion of reaction the mixture is cooled to ambient temperature and filtered through a pad of diatomite (4.1 kg). The filter cake is washed with EtOAc (3×65 L). The combined filtrate and washing liquids are stirred with activated charcoal (8.1 kg) at ambient temperature for 2 h. The decolorized solution is filtered, and the filter cake is washed with EtOAc (61 L). The combined filtrate and washing liquids are concentrated under vacuum to approx. 60 L, after which a solvent switch to EtOH is performed by repeated addition of EtOH (61 L) and concentration to 60 L. Then, more EtOH (36 L) is added, and the resulting mixture is heated at reflux for about 30 min, cooled down to ambient temperature, stirred overnight, and filtered. The filter cake is washed with EtOH (20 L) and dried under vacuum at 50° C. to afford INT 1e as a light-yellow solid (13.3 kg) in 74% yield.

### Step 2: Synthesis of 2-Amino-1-(3-methoxy-2,6-dimethylphenyl)-5,6-dimethyl-1H-pyrrolo[2,3-b]-pyridine-3-carbonitrile (INT 1f)

To a solution of malononitrile (12.8 kg, 194 mol) in DME (620 L) is added portion wise NaO-t-Bu (17.6 kg, 183 mol) at 0° C. This is followed by addition of INT 1e (30.9 kg, 92.2 mol) and Pd(dppf)Cl₂·CH₂—Cl₂ (4.01 kg, 4.91 mol) at ambient temperature. The resulting mixture is heated at reflux for 18 h. After completion of reaction the reaction mass is cooled to 35° C. and filtered through a pad of diatomite (30.9 kg). The filter cake is washed with DME (3×180 L). The combined filtrate and washing liquids are stirred with activated charcoal (30.9 kg) at 60° C. for 1.5 h. The decolorized solution is filtered, and the filter cake is washed with DME (2×107 L). The combined filtrate and washing liquids are subjected to a second decolorization with activated charcoal (30.9 kg) at 60° C. but washing of the filter cake is this time performed with EtOAc (2×103 L). The combined filtrate and washing liquids are concentrated under vacuum to 240 L, after which a solvent switch to i-PrOH is performed by repeated addition of i-PrOH (197 L) and concentration to 240 L. The resulting mixture is heated at reflux for about 30 min, cooled down to ambient temperature, stirred overnight, and filtered. The filter cake is washed with i-PrOH (79 L) and dried under vacuum at 50° C. to afford INT 1f as a light-yellow solid (21.3 kg) in 72% yield.

### Step 3: Synthesis of (S)-2-Ammonium-1-(3-methoxy-2,6-dimethylphenyl)-5,6-dimethyl-1H-pyrrolo[2,3-b]-pyridine-3-carbonitrile/(R)-CSA ((S)-INT 1f/(R)-CSA)

A mixture of INT 1f (21.0 kg, 65.7 mol), (R)-CSA (15.4 kg, 66.3 mol), and anisole (612 L) is heated at 85° C. for 1 h and cooled to 50° C., after which crystal seeds (42 g, 0.13 mol) are added. The mixture is stirred at 50° C. for 14 h, cooled to 10° C. over a period of 6 h, aged at this temperature for 16 h, and filtered. The filter cake is washed with anisole (53 L) and dried on the centrifuge for 1 h to afford crude, wet (S)-INT 1f/(R)-CSA (17.2 kg). A suspension of crude(S)-INT 1f/(R)-CSA in anisole (302 L) is heated at 85° C. for 2 h, cooled to 10° C. over a period of 10 h, aged at this temperature for 16 h, and filtered. The filter cake is washed with anisole (43 L) and dried under vacuum at 50° C. to afford (S)-INT 1f/(R)-CSA with a chiral purity of 97.2% as an off-white solid (14.4 kg) in 40% yield.

### Step 4: Synthesis of (S)-2-amino-1-(3-hydroxy-2,6-dimethylphenyl)-5,6-dimethyl-1H-pyrrolo[2,3-b]pyridine-3-carboxamide ((S)-Compound 1)

To a mixture of methanesulfonic acid (32 L) and water (0.48 L, total water content 1.60% w/w) is added(S)-INT 1f/(R)-CSA (6.76 kg, 24.6 mol) in portions at 45° C. Stirring is continued at this temperature for 4 h. After completion of reaction, the resulting mass is cooled to ambient temperature and DL-methionine (14.8 kg, 99.2 mol) is added. The reaction mixture is stirred at 75° C. for 18 h, and cooled to ambient temperature, and quenched by drop wise addition to chilled water (141 L) keeping the temperature below 20° C. The quenched mixture is diluted with water (51 L) and stirred with activated charcoal (0.70 kg) at ambient temperature for 3 h. The decolorized solution is filtered through a pad of diatomite (3.4 kg), and the filter cake is washed with water (69 L). Then, 2-MeTHF (30 L) is added to the combined filtrate and washing liquid. The pH of the biphasic system is adjusted to 8-9 with aqueous NaOH (30% w/w, approx. 50 L). The suspension that is obtained is heated at 55° C. for 30 min, cooled to ambient temperature, stirred for 10 h, and filtered. The filter cake is washed with water (3×20 L), dried under vacuum at 50° C. to afford crude(S)-Compound 1 as a light-brown solid (3.75 kg, chemical purity 98.8% area, chiral purity 97.3%) in 94% yield.

Crude (S)-Compound 1 (7.40 kg, 22.8 mol) is dissolved in methanol (93 L) at 65° C., the resulting mixture is cooled to 55° C. and filtered (warm) to remove insoluble material. The filter cake is washed with methanol (19 L). Water (3.7 L) is added to the filtrate at 55° C., which is then cooled to 5° C. Seed crystals (74 g as a suspension in methanol/water 1:1 v/v (8 L)) are added and the subsequent mixture is stirred at 0° C. for 3 h. Water (109 L) is dosed over a period of several hours, after which stirring is continued at 0° C. for 10 h. The resulting suspension is filtered. The filter cake is washed with methanol/water (1:1 v/v, 6 L) and dried under vacuum at 50° C. to afford (S)-Compound 1 as a light-yellow solid (6.11 kg, chemical purity 99.6% area, chiral purity 99.8%) in 83% yield from crude(S)-Compound 1.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of preparing an enantioenriched Compound 1: 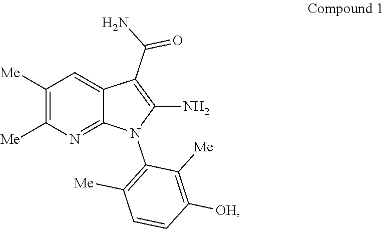 [SMILES: Cc1cc2c(C(N)=O)c(N)n(-c3c(C)ccc(O)c3C)c2nc1C]
or a salt thereof,
the method comprising the steps of
(a) producing an enantioenriched chiral salt of compound INT 1f: 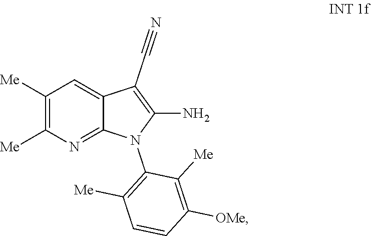 [SMILES: COc1ccc(C)c(-n2c(N)c(C#N)c3cc(C)c(C)nc32)c1C]
and
(b) converting the enantioenriched chiral salt of compound INT 1f to Compound 1 or a salt thereof.

2. The method of claim 1, wherein step (a) comprises reacting compound INT 1f with an enantioenriched chiral acid.

3. The method of claim 2, wherein the enantioenriched chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; (1S)-(+)-10-Camphorsulfonic acid; (+)-2,3-dibenzoyl-D-tartaric acid; (−)-2,3-dibenzoyl-L-tartaric acid; (−)-O,O′-di-p-toluoyl-L-tartaric acid; (+)-O,O′-di-p-toluoyl-D-tartaric acid; (S)-TCYP; (R)-TCYP; (S)-TRIP; or (R)-TRIP, and the enantioenriched chiral salt is a corresponding chiral acid addition salt of compound INT 1f.

4. The method of claim 3, wherein the enantioenriched chiral acid is (1R)-(−)-10-camphorsulfonic acid, and the enantioenriched chiral salt is a (1R)-(−)-10-camphorsulfonic acid salt of compound INT 1f.

5. The method of any one of claims 2 to 4, wherein step (a) comprises reactive crystallization from anisole.

6. The method of any one of claims 1 to 5, wherein step (b) comprises hydrolyzing the enantioenriched salt of compound INT 1f to produce an enantioenriched compound INT 1g: 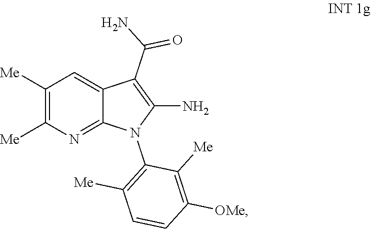 [SMILES: COc1ccc(C)c(-n2c(N)c(C(N)=O)c3cc(C)c(C)nc32)c1C]
or a salt thereof.

7. The method of claim 6, wherein the hydrolyzing step comprises reacting compound INT 1f with a strong Brønsted acid.

8. The method of claim 7, wherein the strong Brønsted acid is methanesulfonic acid.

9. The method of any one of claims 6 to 8, wherein step (b) comprises deprotecting compound INT 1g or a salt thereof to produce Compound 1 or a salt thereof.

10. The method of claim 9, wherein the deprotecting step comprises reacting compound INT 1f with methionine.

11. The method of any one of claims 6 to 10, wherein INT 1g is (S)-INT 1g: 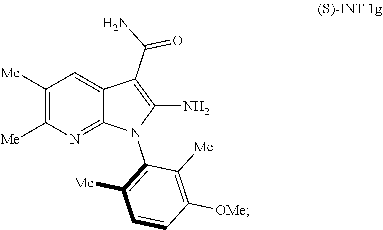
wherein INT 1f is (S)-INT 1f: 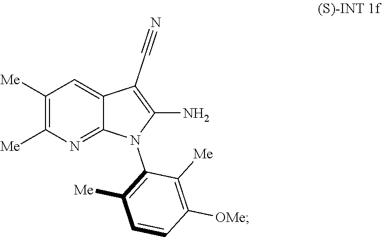
and wherein Compound 1 is (S)-Compound 1:

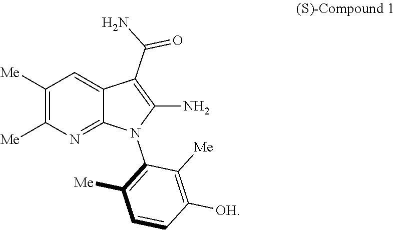

12. The method of any one of claims 1 to 11, wherein the enantioenriched chiral acid is at least 90% ee.

13. The method of any one of claims 1 to 12, wherein the enantioenriched Compound 1 or a salt thereof is at least 98% ee.

14. The method of any one of claims 1 to 13, wherein step (b) comprises recrystallization.

15. The method of claim 14, wherein recrystallization is performed from a methanol/water solvent system.

16. A method of preparing an enantioenriched Compound 1, or a salt thereof, the method comprising the steps of
(a) producing an enantioenriched chiral salt of compound INT 1g; and
(b) converting the enantioenriched chiral salt of compound INT 1g to Compound 1 or a salt thereof.

17. The method of claim 16, wherein step (a) comprises reacting compound INT 1g with an enantioenriched chiral acid.

18. The method of claim 17, wherein the enantioenriched chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; (1S)-(+)-10-Camphorsulfonic acid; (+)-2,3-dibenzoyl-D-tartaric acid; (−)-2,3-dibenzoyl-L-tartaric acid; (−)-O,O′-di-p-toluoyl-L-tartaric acid; (+)-O,O′-di-p-toluoyl-D-tartaric acid; (S)-TCYP; (R)-TCYP; (S)-TRIP; or (R)-TRIP, and the enantioenriched chiral salt is a corresponding chiral acid addition salt of compound INT 1g.

19. The method of claim 18, wherein the enantioenriched chiral acid is (1R)-(−)-10-camphorsulfonic acid, and the enantioenriched chiral salt is a (1R)-(−)-10-camphorsulfonic acid salt of compound INT 1g.

20. The method of any one of claims 17 to 19, wherein step (a) comprises reactive crystallization from anisole.

21. The method of any one of claims 16 to 20, wherein step (b) comprises deprotecting compound INT 1g or a salt thereof to produce Compound 1 or a salt thereof.

22. The method of claim 21, wherein the deprotecting step comprises reacting compound INT 1g with methionine.

23. The method of any one of claims 16 to 22, wherein INT 1g is (S)-INT 1g, and wherein Compound 1 is (S)-Compound 1.

24. The method of any one of claims 16 to 23, wherein the enantioenriched chiral acid is at least 90% ee.

25. The method of any one of claims 16 to 24, wherein the enantioenriched Compound 1 or a salt thereof is at least 90% ee.

26. The method of any one of claims 16 to 25, wherein the step of converting the enantioenriched chiral salt of compound INT 1g to Compound 1 or a salt thereof comprises recrystallization.

27. The method of claim 26, wherein recrystallization is performed from a methanol/water solvent system.

28. A method of preparing an enantioenriched Compound 1, the method comprising the steps of
(a) producing an enantioenriched chiral salt of Compound 1; and
(b) converting the enantioenriched chiral salt of Compound 1 to Compound 1.

29. The method of claim 28, wherein step (a) comprises reacting Compound 1 with an enantioenriched chiral acid.

30. The method of claim 29, wherein the enantioenriched chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; (1S)-(+)-10-Camphorsulfonic acid; (+)-2,3-dibenzoyl-D-tartaric acid; (−)-2,3-dibenzoyl-L-tartaric acid; (−)-O,O′-di-p-toluoyl-L-tartaric acid; (+)-O,O′-di-p-toluoyl-D-tartaric acid; (S)-TCYP; (R)-TCYP; (S)-TRIP; or (R)-TRIP, and the enantioenriched chiral salt is a corresponding chiral acid addition salt of Compound 1.

31. The method of claim 30, wherein the enantioenriched chiral acid is (1R)-(−)-10-camphorsulfonic acid, and the enantioenriched chiral salt is a (1R)-(−)-10-camphorsulfonic acid salt of Compound 1.

32. The method of any one of claims 29 to 31, wherein step (a) comprises reactive crystallization from anisole.

33. The method of any one of claims 28 to 32, wherein Compound 1 is (S)-Compound 1.

34. The method of any one of claims 28 to 33, wherein the enantioenriched chiral acid is at least 90% ee.

35. The method of any one of claims 28 to 34, wherein the enantioenriched Compound 1 or a salt thereof is at least 90% ee.

36. The method of any one of claims 28 to 35, wherein step (b) comprises recrystallization.

37. The method of claim 36, wherein recrystallization is performed from a methanol/water solvent system.

38. A chiral acid addition salt of compound INT 1f, wherein the chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; (1S)-(+)-10-Camphorsulfonic acid; (+)-2,3-dibenzoyl-D-tartaric acid; (−)-2,3-dibenzoyl-L-tartaric acid; (−)-O,O′-di-p-toluoyl-L-tartaric acid; (+)-O,O′-di-p-toluoyl-D-tartaric acid; (S)-TCYP; (R)-TCYP; (S)-TRIP; or (R)-TRIP.

39. The chiral acid addition salt of claim 38, wherein the chiral acid is (1R)-(−)-10-camphorsulfonic acid.

40. The chiral acid addition salt of claim 38 or 39, wherein compound INT 1f is compound(S)-INT 1f.

41. The chiral acid addition salt of any one of claims 38 to 40, wherein the enantioenrichment of compound INT 1f is at least 90% ee.

42. A chiral acid addition salt of compound INT 1g, wherein the chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; (1S)-(+)-10-Camphorsulfonic acid; (+)-2,3-dibenzoyl-D-tartaric acid; (−)-2,3-dibenzoyl-L-tartaric acid; (−)-O,O′-di-p-toluoyl-L-tartaric acid; (+)-O,O′-di-p-toluoyl-D-tartaric acid; (S)-TCYP; or (R)-TCYP.

43. The chiral acid addition salt of claim 42, wherein the chiral acid is (1R)-(−)-10-camphorsulfonic acid.

44. The chiral acid addition salt of claim 42 or 43, wherein compound INT 1g is (S)-INT 1g.

45. The chiral acid addition salt of any one of claims 42 to 44, wherein the enantioenrichment of compound INT 1g is at least 90% ee.

46. A chiral acid addition salt of Compound 1, wherein the chiral acid is (S)-(+)-1,1′-Binaphthyl-2,2′-diyl hydrogenphosphate; (R)-(−)-1,1′-Binaphthyl-2,2′-diyl hydrogenphosphate; (1S)-(+)-3-Bromocamphor-10-sulfonic acid hydrate; (1R)-(−)-10-camphorsulfonic acid; or (1S)-(+)-10-Camphorsulfonic acid.

47. The chiral acid addition salt of claim 46, wherein the chiral acid is (1R)-(−)-10-camphorsulfonic acid.

48. The chiral acid addition salt of claim 46 or 47, wherein Compound 1 is (S)-Compound 1.

49. The chiral acid addition salt of any one of claims 46 to 48, wherein the enantioenrichment of compound 1 is at least 90% ee.
